SEQUENCE LISTING (16 sequences)

SEQ ID NO: 1 - protein, 7 aa, Escherichia coli
APAVNVG

SEQ ID NO: 2 - protein, 14 aa, Escherichia coli
TPVSSAGGVAIKAG

SEQ ID NO: 3 - protein, 11 aa, Escherichia coli
ANNDVVVPTGG

SEQ ID NO: 4 - protein, 8 aa, Escherichia coli
APAVNVGQ

SEQ ID NO: 5 - protein, 15 aa, Escherichia coli
TPVSSAGGVAIKAGS

SEQ ID NO: 6 - protein, 8 aa, Artificial Sequence
  Artificial peptide of SEQ ID NO4 with an amino acid substitution
APAVNVGL

SEQ ID NO: 7 - protein, 15 aa, Artificial Sequence
  Artificial peptide of SEQ ID NO5 with an amino acid substitution
TPVSSAGGVAIKAGA

SEQ ID NO: 8 - protein, 11 aa, Artificial Sequence
  Artificial peptide of SEQ ID NO3 with an amino acid substitution
ANNDPVVPTGG

SEQ ID NO: 9 - protein, 11 aa, Artificial Sequence
  Artificial peptide of SEQ ID NO3 with an amino acid substitution
ANNDVPVPTGG

SEQ ID NO: 10 - protein, 11 aa, Artificial Sequence
  Artificial peptide of SEQ ID NO3 with an amino acid substitution
ANNDVVPPTGG

SEQ ID NO: 11 - protein, 11 aa, Artificial Sequence
  Artificial peptide of SEQ ID NO3 with an amino acid substitution
ANNDPPVPTGG

SEQ ID NO: 12 - protein, 11 aa, Artificial Sequence
  Artificial peptide of SEQ ID NO3 with an amino acid substitution
ANNDVPPPTGG

SEQ ID NO: 13 - protein, 11 aa, Artificial Sequence
  Artificial peptide of SEQ ID NO3 with an amino acid substitution
ANNDPPPPTGG

SEQ ID NO: 14 - protein, 300 aa, Escherichia coli
MKRVITLFAVLLMGWSVNAWSFACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 15 - protein, 279 aa, Escherichia coli
FACKTANGTAIPIGGGSANVYVNLAPAVNVGQNLVVDLSTQIFCHNDYPETITDYVTLQRGSAYGGVLSSFSGTVKYNGSSYPFPTTSETPRVVYNSRTDKPWPVALYLTPVSSAGGVAIKAGSLIAVLILRQTNNYNSDDFQFVWNIYANNDVVVPTGGCDVSARDVTVTLPDYPGSVPIPLTVYCAKSQNLGYYLSGTTADAGNSIFTNTASFSPAQGVGVQLTRNGTIIPANNTVSLGAVGTSAVSLGLTANYARTGGQVTAGNVQSIIGVTFVYQ

SEQ ID NO: 16 - DNA, 903 bp, Artificial Sequence
  Artificial DNA sequence of a FimH gene
atgaaacgagttattaccctgtttgctgtactgctgatgggctggtcggtaaatgcctggtcattcgcctgtaaaaccgccaatggtaccgcaatccctattggcggtggcagcgccaatgtttatgtaaaccttgcgcctgccgtgaatgtggggcaaaacctggtcgtagatctttcgacgcaaatcttttgccataacgattacccagaaaccattacagactatgtcacactgcaacgaggttcggcttatggcggcgtgttatctagtttttccgggaccgtaaaatataatggcagtagctatcctttccctactaccagcgaaacgccgcgggttgtttataattcgagaacggataagccgtggccggtggcgctttatttgacgccggtgagcagtgcggggggagtggcgattaaagctggctcattaattgccgtgcttattttgcgacagaccaacaactataacagcgatgatttccagtttgtgtggaatatttacgccaataatgatgtggtggtgcccactggcggctgcgatgtttctgctcgtgatgtcaccgttactctgccggactaccctggttcagtgccgattcctcttaccgtttattgtgcgaaaagccaaaacctggggtattacctctccggcacaaccgcagatgcgggcaactcgattttcaccaataccgcgtcgttttcacccgcgcagggcgtcggcgtacagttgacgcgcaacggtacgattattccagcgaataacacggtatcgttaggagcagtagggacttcggcggtaagtctgggattaacggcaaattacgcacgtaccggagggcaggtgactgcagggaatgtgcaatcgattattggcgtgacttttgtttatcaataa

(19) United States
(12) Patent Application Publication
(10) Pub. No.: US 2004/0067544 A1
(43) Pub. Date: Apr. 8, 2004

(54) Use of adhesion molecules as bond stress-enhanced nanoscale binding switches

(76) Inventors: Viola Vogel, Seattle, WA (US); Wendy Thomas, Seattle, WA (US); Manu Forero, Seattle, WA (US); Evgeni Sokurenko, Seattle, WA (US)

Correspondence Address: GREENLEE WINNER AND SULLIVAN P C, 5370 MANHATTAN CIRCLE, SUITE 201, BOULDER, CO, 80303, US

(21) Appl. No.: 10/607,834

(22) Filed: Jun. 27, 2003

Related U.S. Application Data
(60) Provisional application No. 60/392,467, filed on Jun. 27, 2002.

(51) Int. Cl. C12Q 1/68; G01N 33/554; G01N 33/569

(52) U.S. Cl. 435/7.32

(57) ABSTRACT

Methods, compositions and devices are provided based on changing the binding strength of an adhesion molecule to a ligand by changing the force exerted on the bound complex between adhesion molecule and ligand, for example by changing the shear stress acting on the complex. The adhesion molecules and their ligands of this invention bind more tightly when a force-activated bond stress, such as shear force, applied to the adhesion molecules is increased, and bond less tightly when the stress is decreased. The adhesion molecules can be isolated from their sources in nature or can remain attached to their natural sources. They can be engineered, e.g., by altering their amino acid sequences or by binding to antibodies or other particles, to alter their binding properties. They can be attached to a wide range of substrates including particles and device surfaces to form adhesive systems which are capable of sticking to other particles and/or device surfaces to which ligands for the adhesion molecules have been attached. The adhesion molecules and ligands described herein can be used to control binding and release of components of an adhesive system by increasing or decreasing the force-activated bond stresses applied to the adhesion molecules.

### CROSS-REFERENCE TO RELATED APPLICATIONS

This application claims priority to U.S. Ser. No. 60/392,467 filed Jun. 27, 2002, which is incorporated herein by reference to the extent not inconsistent herewith.

### STATEMENT OF GOVERNMENT FUNDING

This invention was made at least in part using government funding. The U.S. Government may have rights herein.

### BACKGROUND

The bonding strength of glue typically weakens if a tensile mechanical force or a shear stress is applied. The same is true for most receptor-ligand interactions in biology where a tensile force or a shear stress reduces the lifetime of the bound state. Surface adhesion of bacteria generally occurs in the presence of shear stress, and the lifetime of receptor bonds is expected to be shortened in the presence of external force.

Evolution gave Escherichia coli a set of sophisticated tools to adhere and colonize host tissues which is also a key element in the infectious pathway (Soto, G. E. & Hultgren, S. J. (1999)) and the formation of biofilms (Schembri, M. A. & Klemm, P., (2001)). The low-Man1 binding, shear-dependent FimH variants are predominant among E. coli. In the course of infection or colonization, for example, bacteria commonly adhere to host cells or medical implants through specific adhesin-receptor interactions. There they are exposed to and must resist vigorous shear stress imposed by flow of fluids such as mucosal secretions (0.8 dynes/cm² for saliva), or blood (up to 10 dynes/cm²), that are presumed to act as a natural defense against bacterial colonization.

Type I fimbriae are a group of hair-like appendages on the bacterial surface that mediate mannose-sensitive adhesion to host cells. They are the most common type of bacterial adhesins described so far and are expressed by both commensal and pathogenic strains of enterobacteria and by some other families. Type I fimbriae, also known as pili, are the most common organelles that mediate surface attachment between E. coli and its hosts. They are 6-8 nm thick hair-like filaments protruding from the surface of E. coli with an adhesin on their tip that specifically binds to carbohydrates. The helical rod is polymerized from FimA monomers to a total length of up to 2 μm. The tip of this rod consists of the FimF, FimG, and the terminal FimH subunit. The latter is a ˜2 nm lectin that binds preferentially monomannose and oligomannose. In E. coli, Type I fimbriae consist primarily of the FimA structural protein (Brinton, 1965) and terminate in a small tip structure that contains FimF, FimG, and the 30 kDa lectin-like adhesin FimH (Abraham et al., 1987; Hanson et al., 1988; Klemm and Christiansen, 1987). The FimH adhesin consists of a mannose binding lectin domain and a pilin domain that integrates FimH into the fimbrial tip (Choudhury et al., 1999). The amino acid sequence of the FimH variants expressed by different E. coli is on average 99% conserved, and all type I fimbriated E. coli are able to bind strongly to receptors containing trimannose structures (Sokurenko et al., 1997, 1998). At the same time, FimH adhesin of most intestinal E. coli strains does not mediate strong binding to receptors that contain primarily monomannose (Man1) terminal residues (Sokurenko et al., 1995, 1997, 1998). However, many FimH variants of uropathogenic E. coli origin have a relatively high Man1 binding capability due to the presence of functional point mutations at various positions in the FimH molecule (Schembri et al., 2000; Sokurenko et al., 1995, 1998).

The main purpose of receptor-specific adhesion of bacteria is to prevent detachment from the target surface. In the course of infection or colonization, for example, bacteria commonly adhere to host cells or medical implants through specific adhesin-receptor interactions (Beachey, 1981; Gibbons, 1984). There they are exposed to and must resist vigorous shear stress imposed by flow of fluids such as mucosal secretions (0.8 dynes/cm² for saliva) or blood (up to 10 dynes/cm²) that are presumed to act as a natural defense against bacterial colonization (Christersson et al., 1988; Dickinson et al., 1995, 1997; Pratt and Kolter, 1998; Pratt-Terpstra et al., 1987; Shive et al., 1999; Wang et al., 1995).

Therefore, it would be beneficial for bacteria to be able to modulate the binding strength of adhesins under variable shear. Some studies have suggested that bacteria-surface interactions might be enhanced by shear (Brooks et al., 1989; Brooks and Trust, 1983a, 1983b; Li et al., 2000; Mohamed et al., 2000). However, it has not been shown directly whether and how functional properties of bacterial adhesins are directly modulated by shear.

It is an object of this invention to show that shear-induced mechanical force enhances the strength of receptor-specific interactions between adhesion molecules such as FimH and target cells, and that this phenomenon is dependent on the structural properties of the adhesion molecules.

Methods, compositions and devices for using shear-dependent binding in a variety of applications would be extremely useful in the biomedical and other fields.

All references cited herein are incorporated by reference to the extent not inconsistent herewith.

Abraham, S. N. et al. (1987) “Identification of two ancillary subunits of Escherichia coli type I fimbriae by using antibodies against synthetic oligopeptides of fim gene products,” J. Bacteriol. 169:5530-5536.

Alon, R., et al. (1997) “The kinetics of L-selectin tethers and the mechanics of selectin-mediated rolling,” J. Cell Biol. 138:1169-1180.

Bass, R. B. et al. (2002) “Crystal structure of Escherichia coli MscS, a voltage-modulated and mechanosensitive channel,” Science 298:1582-1587.

Beachey, E. H. (1981) “Bacterial adherence: adhesin-receptor interactions mediating the attachment of bacteria to mucosal surface,” J. Infect. Dis. 143:325-345.

Beck, R. and Burtscher, H. (1994) “Introduction of arbitrary sequences into genes by use of class IIs restriction enzymes,” Nucleic Acids Res. 22:886-887.

Bell, G. I. (1978) “Models for the specific adhesion of cells to cells,” Science 200:618-627.

Bloch, C. A. et al. (1992) “A key role for type 1 pili in enterobacterial communicability,” Mol. Microbiol. 6:697-701.

Brinton, C. (1965) “The structure, function, synthesis and genetic control of bacterial pili and a molecular model for DNA and RNA transport in gram-negative bacteria,” Trans. N.Y. Acad. Sci. 27:1003-1054.

Brooks, D. E. et al. (1989) “Involvement of the MN blood group antigen in shear-enhanced hemagglutination induced by the Escherichia coli F41 adhesin,” Infect. Immun. 57:377-383.

Brooks, D. E. and Trust, T. J. (1983) “Enhancement of bacterial adhesion by shear forces: characterization of the haemagglutination induced by Aeromonas salmonicida strain 438,” J. Gen. Microbiol. 129:3661-3669.

Brooks, D. E. and Trust, T. J. (1983) “Interactions of erythrocytes with bacteria under shear” Ann. NY Acad. Sci. 416:319-331.

Campbell, J. J. et al., (Jan. 16, 1998) Science 279:381-384.

Celikel, R. et al. (2000) “von Willebrand factor conformation and adhesive function is modulated by an internalized water molecule,” Nat. Struct. Biol. 7:881-884.

Chang, K. C. et al., (2000) Proc. Natl. Acad. Sci. USA 97:11262-11267.

Chen, S. and Springer, T. A. (2001) “Selectin receptor-ligand bonds: formation limited by shear rate and dissociation governed by the Bell model,” Proc. Natl. Acad. Sci. USA 98:950-955.

Choudhury, D. et al. (1999) “X-ray structure of the FimC-FimH chaperone-adhesin complex from uropathogenic Escherichia coli,” Science 285:1061-1066.

Christersson, C. E. et al. (1988) “Role of temperature and shear forces on microbial detachment,” Scand. J. Dent. Res. 96:91-98.

Dembo, M. D. et al., (1988) Proc. R. Soc. Lond. B. Biol. Sci. 234:55-83

Dickinson, R. B. et al. (1997) “Quantitative comparison of shear-dependent Staphylococcus aureus adhesion to three polyurethane ionomer analogs with distinct surface properties,” J. Biomed. Mater. Res. 36:152-162.

Dickinson, R. B. et al. (1995), “Quantitative comparison of clumping factor- and coagulase-mediated Staphylococcus aureus adhesion to surface-bound fibrinogen under flow,” Infect. Immun. 63:3143-3150.

Evans, E. (2001) Annu. Rev. Biophys. Biomol. Struct. 30:105-28.

Evans, E. (1999) “Looking inside molecular bonds at biological interfaces with dynamic force spectroscopy,” Biophys. Chem. 82:83-97.

Evans, E. et al. (1996) “Biomembrane templates for nanoscale conduits and networks,” Science 273:933-935.

Finger, E. B. et al. (1996) “Adhesion through L-selectin requires a threshold hydrodynamic shear,” Nature 379:266-269.

Gibbons, R. J. (1984) “Adherent interactions which may affect microbial ecology in the mouth,” J. Dent. Res. 63:378-385.

Goldman, A. J. et al. (1967) Chem. Eng. Sci. 22:653-660.

Hanson, M. S. et al. (1988) “Purification of the Escherichia coli type 1 pilin and minor pilus proteins and partial characterization of the adhesin protein,” J. Bacteriol. 170:3350-3358.

Humphrey, W. et al. (1996) “Vmd: visual molecular dynamics,” J. Mol. Graph. 14:33-38.

Isralewitz, B. et al. (2001) “Steered molecular dynamics and mechanical functions of proteins,” Curr. Opin. Struct. Biol. 11:224-230.

Johnson, J. R. (1991) “Virulence factors in Escherichia coli urinary tract infection,” Clin. Microbiol Rev. 4:80-128

Jones, C. H. et al. (1995) “FimH adhesion of type 1 pili is assembled into a fibrillar tip structure in the enterobacteriaceae,” Proc. Natl. Acad. Sci. USA 92(6):2081-2085

Kale, L. et al. (1999) “NAMD2: greater scalability for parallel molecular dynamics,” J. Comput. Phys. 151:283-312.

Keurin et al. (May 2003) J. Lab. Clin. Med. 141(5):350-358

Klemm, P. and Christiansen, G. (1987) “Three fim genes required for the regulation of length and mediation of adhesion of Escherichia coli type I fimbriae,” Mol. Gen. Genet. 208:439-445.

Krammer, A. et al. (2002) “A structural model for force regulated integrin binding to fibronectin's RGD-synergy site,” Matrix Biol. 21:139-147.

Langermann, S. et al. (1997) “Prevention of mucosal Escherichia coli infection by FimH-adhesin-based systemic vaccination,” Science 276(5312):607-611.

Lawrence, M. B. et al. (1997), J Cell Biol. 136:717-2711.

Li, Z. J. et al. (2000) “Shear stress affects the kinetics of Staphylococcus aureus adhesion to collagen,” Biotechnol. Prog. 16:1086-1090.

Marchese, P. et al. (1999) “Adhesive properties of the isolated amino-terminal domain of platelet glycoprotein Ib alpha in a flow field,” Proc. Natl. Acad. Sci. USA 96:7837-7842.

Marshall, B. T. et al. (2003) “Direct observation of catch bonds involving cell-adhesion molecules,” Nature 423:190-193.

Mascari, L. and Ross, J. M. (2001) “Hydrodynamic shear and collagen receptor density determine the adhesion capacity of S-aureus to collagen,” Annals of Biomedical Engineering 29:956-962

Merkel, R. et al. (1999) “Energy landscapes of receptor-ligand bonds explored with dynamic force spectroscopy,” Nature 397:50-53.

Mohamed, N. et al. (2000) “Novel experimental study of receptor-mediated bacterial adhesion under the influence of fluid shear,” Biotechnol. Bioeng. 68:628-636.

Pratt, L. A. and Kolter, R. (1998) “Genetic analysis of Escherichia coli biofilm formation: roles of flagella, motility, chemotaxis and type I pili,” Mol. Microbiol. 30:285-293.

Pratt-Terpstra, I. H. et al. (1987) “Adhesion of oral streptococci from a flowing suspension to uncoated and albumin-coated surfaces,” J. Gen. Microbiol. 133:3199-3206.

Schembri, M. A. and Klemm, P. (2001) “Biofilm formation in a hydrodynamic environment by novel FimH variants and ramifications for virulence,” Infect. Immun. 69:1322-1328

Schembri, M. A. et al. (2000) “Functional flexibility of the FimH adhesin: insights from a random mutant library,” Infect. Immun. 68:2638-2646.

Shive, M. S. et al. (1999) “Shear stress effects on bacterial adhesion, leukocyte adhesion, and leukocyte oxidative capacity on a polyetherurethane,” J. Biomed. Mater. Res. 46:511-519.

Sokurenko, E. V. et al. (2001) “Valency conversion in the type I fimbrial adhesin of Escherichia coli,” Mol. Microbiol. 41:675-686.

Sokurenko, E. V. et al. (1998) “Pathogenic adaptation of Escherichia coli by natural variation of the FimH adhesion,” Proc. Natl. Acad. Sci. USA 95:8922-8926.

Sokurenko, E. V. et al. (1997), “Diversity of the Escherichia coli type I fimbrial lectin. Differential binding to mannosides and uroepithelial cells,” J. Biol. Chem. 272:17880-17886.

Sokurenko, E. V. et al. (1995) “Quantitative differences in adhesiveness of type I fimbriated Escherichia coli due to structural differences in FimH genes,” J. Bacteriol. 177:3680-3686.

Sokurenko, E. V. et al. (1994) “FimH family of type I fimbrial adhesins: functional heterogeneity due to minor sequence variations among FimH genes,” J. Bacteriol. 176:748-755

Soto, G. E. and Hultgren, S. J. (1999) “Bacterial adhesins: common themes and variations in architecture and assembly,” J. Bacteriol. 181:1059-1071.

Sowdhamini, R. et al. (1989) “Stereochemical modeling of disulfide bridges. Criteria for introduction into proteins by site-directed mutagenesis,” Protein Eng. 3:95-103.

Theilmeier, G. et al. (Jun. 15 2002) “Endothelial von Willebrand factor recruits platelets to atherosclerosis-prone sites in response to hypercholesterolemia,” Blood 99(12):4486-4493.

Thomas, W. E. et al. (Jun. 28, 2002) “Bacterial adhesion to target cells enhanced by shear force,” Cell 109:913-23.

Vogel, V. et al. (2001) “Structural insights into the mechanical regulation of molecular recognition sites,” Trends Biotechnol. 19:416-423.

Wang, I. W. et al. (1995) “Adhesion of Staphylococcus epidermidis to biomedical polymers: contributions of surface thermodynamics and hemodynamic shear conditions,” J. Biomed. Mater. Res. 29:485-493.

Wang, K. et al. (2001) “Single molecule measurements of titin elasticity,” Prog. Biophys. Mol. Biol. 77:1-44.

### SUMMARY

This invention provides methods, compositions and devices based on changing the binding strength of an adhesion molecule such as an adhesin or integrin to a ligand such as a mannose by changing the force exerted on the bond, for example by changing the shear stress, and consequently the tensile force, on the bond. In contrast to normal bond behavior, the adhesion molecules and their ligands used in this invention, bind more tightly when a force-activated bond stress, such as shear force or a tensile force, applied to the adhesion molecules is increased, and bond less tightly when the stress is decreased.

This invention also provides adhesion molecules isolated from their sources in nature and attached to a wide range of substrates including particles and device surfaces to form adhesive systems which are capable of sticking to other particles and/or device surfaces to which ligands for the adhesion molecules have been attached. For example films can be coated with one member of an adhesion molecule/ligand pair and can adhere to films or other surfaces coated with the other member of the pair under appropriate bond stress conditions. Or films can be coated with a mixture of adhesion molecules and ligands and become self-adhering under appropriate bond stress conditions.

Binding of adhesion molecules and their ligands can be controlled by using adhesion molecules provided herein which have been engineered to have changed binding properties, e.g., are capable of more efficiently bonding to their ligands under force-activated bond stress, compared to their naturally-occurring counterparts. These molecules include mutated and truncated adhesion molecules. Binding of adhesion molecules and their ligands can also be controlled by attaching antibodies or other molecules or particles to the adhesion molecules which change their ability to respond to changes in applied bond stresses on the molecules. This invention provides antibodies to various adhesion molecules which are useful for this purpose.

The adhesion molecules and ligands described herein can be used to control binding and release of system components by increasing or decreasing the force-activated bond stresses applied to the adhesion molecules.

These molecules and ligands can be used in methods to provide substantially uniform mixtures of complexed particles in a fluid carrier, by attaching adhesion molecules to one type of particle and attaching ligands for the adhesion molecules to another type of particle. The components are then mixed to form a homogenous mixture, and then an appropriate stress is applied, e.g. turbulence is increased, causing the adhesion molecules to bind to their ligands, forming a mixture of complexes which is substantially uniform.

The adhesion molecules and ligands described herein can also be used to form self-assembling geometrical patterns. Selected surfaces of three-dimensional forms, such as cylinders, can be coated with adhesion molecules, and with their ligands, and then the appropriate bond stress can be applied to cause the adhesion molecules to bind to their ligands, thus causing the three-dimensional forms to bond to each other in a desired pattern. The three-dimensional forms can be varied, and different surfaces can be coated, to produce a wide variety of layers and assemblies of these forms.

Certain ligands, because of their size, charge, or other properties, can change the amount of force-activated bond stress an adhesion molecule is receiving under given process conditions. The adhesion molecules described herein can thus also be used for separating ligand molecules (including particles to which they may be bound) which have differing abilities to induce bond stress on an adhesion molecule. The method involves adding adhesion molecules attached to removing agents, such as magnetic beads, to the fluid containing the ligands. The appropriate bond stress is then applied to the system to allow binding of one type of ligand molecules to the exclusion of other types present in the fluid. Then a removing force, such as magnetic field, is applied to separate the bound ligand particles.

Fluidic devices and device components having surfaces coated with the adhesion molecules of this invention are provided herein and can be used for a variety of purposes. Such devices include channels, including microscale or macroscale rectangular and cylindrical channels, parallel plate flow chambers, and cell sorters. These devices can be used to release desired particles into a fluid flowing through the device by changing the bond stress on the adhesion molecules to cause release of the desired particles which have been attached to the devices by means of ligands for the adhesion molecules. The adhesion molecules and ligands described herein can also be used to deliver particles to the surface of a device, by coating the surface with one member of an adhesion molecule/ligand pair and attaching the particles to be delivered to the other member of the pair, then introducing the particles under the appropriate bond stress conditions to cause binding of the particles to the surface of the device.

The adhesion molecules and ligands described herein can also be used to measure the rate of fluid flow in a device by detecting the amount of binding of adhesion molecules and ligands in the device.

The adhesion molecules and ligands described herein can also be used as “valves” to change the rate of flow of a fluid through a device such as a channel by applying appropriate bond stresses to cause clogging and unclogging of the channel or other flow path. The adhesion molecules and ligands should be attached to particles or a combination of particles and surfaces so that shear forces applied to them will be sufficient to cause stress-dependent binding.

The adhesion molecules and ligands described herein can also be used as viscosity modifiers (by themselves or attached to other particles) capable of changing the viscosity of a fluid in response to a change in force-activated bond stress applied to the adhesion molecules. Both the FABSDAM and the FABSDB-L should be attached to a particle such that shear forces applied to them will be sufficient to cause stress-dependent binding.

Over a particular critical range of force-activated bond stress conditions for each adhesion molecule/ligand pair, these pairs, which are capable of bond stress-activated binding, bond more tightly to each other when the bond stress is increased and less tightly when the bond stress is decreased. When the bond stress is still further increased, above this critical range, increased bond stress will decrease binding; however, it will not decrease binding as much as would be expected if the molecules were not capable of bond stress-activated binding. Control of binding by increasing or decreasing bond stress on the adhesion molecules can thus be performed in a novel and unexpected manner above the critical range by changing the bond stress on the molecules.

### BRIEF DESCRIPTION OF THE DRAWINGS

FIGS. 1A-D are graphs showing the movement of red blood cells bound to a carpet of E. coli under bond stress.

FIGS. 2A-B are drawings showing a steered molecular dynamic analysis of FimH.

FIGS. 3A-D are drawings showing a steered molecular dynamic analysis of FimH structural changes occurring in the interdomain region.

FIGS. 4A-B are graphs showing the effects of engineered FimH mutants on the velocity of red blood cells bound to a carpet of E. coli.

FIGS. 5A-B are graphs showing the functional significance of shear activation.

FIGS. 6A-B are graphs showing the accumulation of E. coli on purified receptors.

FIGS. 7A-B are graphs showing the attachment of E. coli on 1Man-BSA surfaces.

FIGS. 8A-D are graphs showing the effects of changes in shear stress on E. coli bound to 1man surfaces.

FIG. 9 is a graph showing the effect of shear on bacterial detachment.

FIGS. 10A-B are graphs showing the effect of shear on the binding properties of red blood cells.

FIG. 11 is a graph showing the velocity of beads covered with different ligands.

FIG. 12 is a graph showing relative particle velocity of particles of different sizes.

FIGS. 13A-D are drawings showing bead movement under different conditions

FIGS. 14A-C are drawings showing alternative designs of receptor/ligand attached particles.

FIGS. 15A-C are drawings of agglutination of red blood cells by E. coli.

FIG. 16 is a drawing showing aggregating and dispersing particles functionalized with adhesins and ligands.

FIGS. 17A-D are three drawings and a graph showing movement of red blood cells.

FIGS. 18A-C are drawings showing assembly of components into geometric patterns.

FIGS. 19A-B are drawings showing microvalves and channels.

FIG. 20 is a drawing showing a valve.

FIGS. 21A-B are graphs showing ligand velocity as a function of bond stress and binding strength as a function of bond stress.

### DETAILED DESCRIPTION

A new method is disclosed for using shear stress or tensile force to enhance the binding of two systems, mediated by biological or engineered adhesins and other adhesion molecules and their respective ligands. The applications include the mannose-binding bacterial adhesin FimH and other receptor-ligand pairs that strengthen under the influence of a force-activated bond stress such as a shear stress or a tensile force. Though shear force normally decreases bond lifetimes, it has been discovered that bacterial attachment to target cells switches from loose to firm under the right shear conditions, which serves as the basis of this invention. This invention allows force-activated and reversible binding of two or more systems via this mechanism, and provides means to block force-activation on demand. This invention has many medical applications, as well as applications in many other fields, including biotechnology, materials sciences, microfluidics, for making and using shear or force-enhanced glues, for making and using dilatant fluids whose viscosity increases with shear, for drug delivery, for vaccine design and more.

The adhesion of Escherichia coli to target surfaces is enhanced by shear force. The E. coli adhesion receptor and ligand, i.e., the fimbriae with the terminal adhesin FimH and carbohydrate monomannose, respectively, have been isolated and immobilized on synthetic surfaces to demonstrate using them as shear-activated nano-glue for technological applications. Shear-enhanced adhesion of beads in fluidic devices and shear-controlled site-directed assembly of nano beads are demonstrated. Other receptor-ligand pairs that also show this catch-bond character and strengthen under shear, include P-selectins (Marshall, B. T. et al. “Direct observation of catch bonds involving cell-adhesion molecules” Nature 423, 190-3 (2003)), may be used in a similar manner.

Using Escherichia coli as an example, we show that the lectin-like adhesin FimH acts as a force sensor that switches from low to high affinity for its ligand in the presence of shear (Thomas et al 2002), a finding that we are exploiting for the fabrication of new materials and devices. E. coli bacteria on 1Man-coated surface can exist in three distinct states, firmly bound, rolling or detached. Shear stress can increase initial accumulation of E. coli on 1Man-coated surfaces by over 100-fold and causes a switch from “slip” to “catch” bond behavior.

FimH is the most common type of bacterial adhesin known (most species of enterobacteria and vibrio possess it). Force-activation is the norm rather than an exception. The force-activated mode of adhesion is not limited to FimH. Force-activated bond stress has also been shown to increase the binding of Staphylococcus aureus bacteria to certain collagen receptors (Li et al., 2000; Mohamed et al., 2000), and to enhance adhesion such as the rolling of lymphocytes on selectins (M. B. Lawrence et al. 1997).

By shearing the fimbriae off the surface of bacteria and adsorbing fimbriae to synthetic surfaces, we have created a cell-free model system and studied in detail the interactions between fimbriae and receptor molecules in a controlled environment and explored their technical applications. The power of this assay using purified fimbriae and monomannose (1Man) conjugated to Bovine Serum Albumin (1Man-BSA) allows varying their density and the molecular composition of the test surfaces Demonstrating force-activated bond stress-enhanced adhesion in a cell-free assay allows this force-activated nano-glue to be used for many practical applications.

This invention provides a method for changing binding strength of an isolated force-activated bond stress-dependent adhesion molecule (I-FABSDAM) to a force-activated bond stress-dependent binding ligand (FABSDB-L) for said I-FABSDAM, said method comprising changing a bond stress on said I-FABSDAM wherein said binding strength increases when said bond stress increases and decreases when said bond stress decreases. Both the I-FABSDAM and the FABSDB-L should be attached to a substrate such as a particle or a surface so that shear forces applied to them will be sufficient to cause stress-dependent binding. Bond stresses useful in the practice of this invention include any force which tends to pull the bond apart, such as shear stresses, stresses resulting from tensile force or shear force, tensile forces, shear stresses causing tensile forces, or a combination of these stresses and forces. Methods known in the art for changing bond stresses are useful in the practice of this invention. When a plurality of FABSDB-Ls or FABSDAMs are attached to a single particle and multiple bonds are formed, larger forces may need to be applied to provide enough bond stress to dissociate all the FABSDB-L-FABSDAM bonds than would be necessary if only a single FABSDB-L/FABSDAM were involved. In the methods of this invention, a FABSDAM can be tightly bound to a FABSDB-L. This invention provides a method for decreasing off-rate (frequency of dissociation of the FABSDB-L and FABSDAM) of a force-activated bond stress-dependent binding ligand (FABSDB-L) from an isolated force-activated bond stress-dependent adhesion molecule (I-FABSDAM), said method comprising changing a bond stress on said I-FABSDAM wherein said off-rate decreases when said bond stress increases and increases when said bond stress decreases.

FABSDAMs useful in the practice of this invention include naturally-occurring and isolated adhesins, selectins, and integrins, and adhesion molecules including members of the immunoglobulin superfamily and syndecans that are capable of binding in a force-activated bond stress-dependent manner that are known to the art and that are as yet to be discovered. Adhesins useful in the practice of this invention include FimH polypeptides and the lectin domains of FimH polypeptides. FimH can be from E. coli. A FimH useful in the practice of this invention has a polypeptide sequence of Genbank Accession Number P08191. FimH polypeptides useful in the practice of this invention include naturally occurring FimH variants and engineered FimH polypeptides containing mutations including mutations affecting the force-activated bond stress-dependent binding properties. Naturally occurring FimH variants include FimHs in E. coli strains f-18 and j-96. Engineered FimH polypeptides include FimH polypeptides having a valine at amino acid position 27, a proline at any of positions 154-156, a leucine at position 32, or an alanine at position 124. FABSDB-Ls useful in the practice of this invention include frutctoses, mannoses including monomannose, trimannose, and oligomannose, and all other FABSDB-Ls that bind to FABSDAMs in a force-activated bond stress-dependent manner.

In the practice of this invention, a FABSDAM or an isolated FABSDAM (I-FABSDAM) and/or a FASBSDB-L can be attached to a particle, including, but not limited to bacterial pili, naturally occurring isolated molecules, synthetic molecules, proteins, polypeptides, organelles, prokaryotic cells to which said FABSDAM is not native, eukaryotic cells to which said I-FABSDAM is not native, viruses, organisms, nanoparticles, microbeads, and microparticles or to a surface selected from the group consisting of cell membranes, other biological membranes, device surfaces and synthetic substrate surfaces. Both a FABSDAM and a FASBSDB-L can be attached to the same particle or surface. Methods for attaching proteins and ligands to particles and surfaces are known in the art.

In the practice of this invention, any amount of bond stress may be applied. Each FABSDAM or isolated FABSDAM has a lower and upper bond stress-dependent threshold specific to it defining a range over which binding strength increases as bond stress increases and descreases as bond stress decreases. The amount of bond stress that is useful in a particular embodiment is specific to each FABSDAM, and may be affected by the FABSDB-Ls, optional particles or substrates, and the system context. In the practice of this invention, bond stresses above the lower threshold are useful for causing force-activated bond stress-dependent binding. Methods for determining the lower and upper thresholds are known in the art. In the practice of this invention, a bond stress can be applied that is between a force-activated bond stress dependent lower threshold and a force-activated bond stress dependent upper threshold of a FABSDAM. Bond stresses useful in the practice of this invention include stresses between about 0.01 dynes/cm² and about 100 dynes/cm², between about 0.05 dynes/cm² and about 20 dynes/cm², between about 0.1 dynes/cm² and about 10 dynes/cm², and between about 0.1 dynes/cm² and about 1 dyne/cm².

The methods of this invention can be applied to a system wherein a first component of said system comprises a plurality of I-FABSDAMs attached to a first object, wherein a second component of said system comprises a plurality of FABSDB-Ls attached to a second object, and wherein said I-FABSDAMs and FABSDB-Ls are capable of binding to each other in a force-activated bond stress-dependent manner, and wherein said method comprises increasing bond stress on said I-FABSDAMs, resulting in said first component changing from being unbound to said second component to being bound to said second component.

The methods of this invention can be applied to a system wherein a first component of said system comprises a plurality of said I-FABSDAMs attached to a first object, wherein a second component of said system comprises a plurality of said FABSDB-Ls attached to a second object, and wherein said I-FABSDAMs and FABSDB-Ls are capable of binding to each other in a force-activated bond stress-dependent manner, and wherein said method comprises decreasing bond stress on said I-FABSDAMS, resulting in said first component changing from being bound to said second component to being unbound from said second component

The methods of this invention can be applied to a system wherein a first component of said system comprises a plurality of I-FABSDAMs attached to first particles, and a second component of said system comprises a plurality of I-FABSDB-Ls attached to second particles, said method comprising homogenously mixing said first and second components, then increasing the bond stress on the system, whereby a substantially uniform material comprising complexes of said first components with said second components is formed. In an embodiment of this invention, the homogenous mixing is performed at a bond stress below the lower force-activated bond stress-dependent binding threshold of said I-FABSDAM. The methods of this invention can also include cross-linking said substantially uniform material once said complexes have been formed by increasing said bond stress. The methods of this invention are useful for making substantially uniform materials from components that are not substantially uniform to begin with due to not being completely homogenized before increasing the bond stress on the system.

The methods of this invention can be applied to a system wherein a first component of said system comprises a plurality of I-FABSDAMs attached to first particles, and a second component of said system comprises a plurality of FABSDB-Ls attached to second particles, said method comprising homogenously mixing said first and second components at a bond stress above the higher force-activated bond stress-dependent binding threshold, then decreasing the bond stress on said system, whereby a substantially uniform material comprising complexes of said first components with said second components is formed. Methods known in the art for homogenously mixing are useful in the practice of this invention.

The methods of this invention are useful to assemble three-dimensional objects from subcomponents. A plurality of I-FABSDAMs are attached to a first selected surface of a plurality of first selected three-dimensional forms, wherein a plurality of FABSDB-Ls are attached to second selected surface of a plurality of second selected three dimensional forms, and the bond stress is increased, resulting in said first and second forms self-assembling into a selected geometric pattern. The first form can be the same as the second form. The first and second forms can be cylinders and the first and second surfaces to which the I-FABSDAMs and FABSDB-Ls are attached are the curved sides of the cylinders. The assembled geometric pattern is a layer composed of the cylinders. The layer can be a synthetic membrane. The first and second forms can also be cylinders, and the surfaces to which the I-FABSDAMs and FABSDB-Ls are applied can be the flat ends of the cylinders. The geometric pattern formed is a chain composed of the cylinders. The first form can have I-FABSDAMs attached thereto but not FABSDB-Ls, and the second form can have FABSDB-Ls attached thereto but not FABSDAMS. In this embodiment an alternating link chain will assemble. When the first and second forms are cylinders, wherein each cylinder comprises a first flat end and a second flat end, wherein said first flat ends are attached to said I-FABSDAMs and said second flat ends are attached to said FABSDB-Ls, the methods of this invention are useful for assembling a directional chain composed of said cylinders. Methods for selecting suitable sub-components for self-assembly of geometric patterns are known to the art or easily determined by one skilled in the art without undue experimentation.

The methods of this invention can be performed in a fluid-containing channel, wherein a plurality of I-FABSDAMs and FABSDB-Ls are attached to particles or surfaces and are present in an amount sufficient to clog the channel when the I-FABSDAMs and FABSDB-Ls are bound to each other. The method comprises changing the bond stress on said I-FABSDAMs whereby the binding strength of said I-FABSDAMs and FABSDB-Ls is changed, whereby the flow rate of said fluid through the channel is changed or the pressure of the fluid in the channel is changed. In the practice of this invention, when the bond stress is increased causing the I-FABSDAMs and FABSDB-Ls to be bound to each other, the flow rate is decreased, and when the bond stress is decreased causing the I-FABSDAMs and FABSDB-Ls to be unbound to each other, the flow rate is increased. If flow is prevented, the pressure of the fluid in the channel is correspondingly increased with increasing bond stress and decreased with decreasing bond stress. In the practice of this invention, the I-FABSDAMs and/or the FABSDB-Ls can be bound to particles or to a wall of the channel. In the practice of this invention, the channel can be in fluid communication with a fluid exit port and a bypass port, wherein changing said bond stress changes the amount of fluid flowing through the exit and bypass ports. In an embodiment of this invention, the channel can be a recirculation channel. Systems using channels, valves, recirculating channels, exit ports, and bypass ports are known in the art and useful in the practice of this invention.

This invention provides a method for removing a target particle from a fluid comprising: (a) adding to said fluid a target particle binding agent, said target particle binding agent being attached to a first member of a FABSDAM/FABSDB-L pair; (b) adding to said fluid the second member of a FABSDAM/FABSDB-L pair attached to a removing agent; (c) allowing said target particle binding agent to bind said target particle; (d) applying a bond stress to said FABSDAM to allow force-activated bond stress-dependent binding of said first pair member and said second pair member, thereby forming a complex comprising said target particle, said target particle binding agent attached to said first pair member, and said second pair member attached to said removing agent; and (e) removing said complex from said fluid. In the practice of this invention, step (e) can comprise a step selected from the group consisting of sedimentation, filtration, bioseparation, applying an electric force, and applying a magnetic force. Methods are known in the art for performing sedimentation, filtration, bioseparation, applying an electric force, and applying a magnetic force and are useful in the practice of this invention. In the practice of this invention, the target particle can be selected from the group consisting of pollutant particles, toxin particles, and drug particles. The target particle-binding agent can be an antibody.

This invention provides a method for separating first FABSDB-Ls from second FABSDB-Ls, wherein said FABSDB-Ls are in a fluid, wherein said FABSDB-Ls are capable of binding to FABSDAMs in a force-activated bond stress-dependent manner, and wherein said first and second FABSDB-Ls induce different bond stresses on said FABSDAM under the same conditions, said method comprising: (a) contacting said fluid with a an amount of said FABSDAMs sufficient to bind substantially all of said first FABSDB-Ls, wherein said FABSDAMs are attached to a removing agent; (b) applying a bond stress to said FABSDAMs sufficient to cause binding of said first FABSDB-Ls to said FABSDAMs to form a complex, said bond stress being insufficient to cause binding of said second FABSDB-Ls to said FABSDAMs; and (c) removing said complex comprising said first FABSDB-Ls, and FABSDAMs and said removing agent from said fluid. In the practice of this invention, the removing agent can consist of particles capable of responding to a removing force. Removing agents are known in the art and are useful in the practice of this invention.

In the practice of this invention, the method for separating first FABSDB-Ls from second FABSDB-Ls can also include: (d) contacting said fluid with said FABSDAMs attached to a removing agent in an amount sufficient to bind to substantially all of said second FABSDB-Ls, including contacting the fluid with more FABSDAMs if necessary; (e) applying a second bond stress to said FABSDAMs sufficient to cause binding of said second FABSDB-Ls to said FABSDAMs to form a second complex; and (f) separating said second complex comprising said second FABSDB-L from said fluid. In the practice of this invention, the second bond stress is selected so as to cause selective binding of said FABSDAMs to said second FABSDB-Ls, to the exclusion of other components in said fluid. In the practice of this invention, the first FABSDB-Ls differ from said second FABSDB-Ls in a characteristic selected from the group consisting of magnetic and electric charge, mass, and three dimensional form. In the practice of this invention, the method for separating first FABSDB-Ls from second FABSDB-Ls can also include (g) a step of covalently-linking said FABSDB-Ls to said removing agent.

This invention provides a fluidic device comprising a surface having a plurality of I-FABSDAMs attached thereto. In the practice of this invention, the surface can be a channel wall or portion thereof. The surface can be a component of a channel, a parallel plate flow chamber, a microfluidic channel, or a cell sorter. Parallel plate flow chambers, a microfluidic channels, and cell sorters are known in the art and are useful in the practice of this invention.

This invention provides a method for selectively releasing into a fluid first FABSDB-Ls from a plurality of FABSDAMs to which first and second FABSDB-Ls are stress-dependently bound, and wherein when said FABSDB-Ls are bound to said FABSDAMs under bond stress, said first and second FABSDB-Ls induce different bond stresses on said FABSDAMs under the same fluid flow conditions, said method comprising: (a) contacting said fluid with said FABSDAMs bound to said SDDB-Ls; and (b) changing the bond stress on said FABSDAMs by an amount sufficient to cause release of said first FABSDB-Ls into said fluid, but insufficient to cause release of said second FABSDB-Ls into said fluid.

This invention provides a method for measuring the rate of flow of a fluid comprising: (a) adding a plurality of FABSDAMs or FABSDB-Ls to said fluid; (b) placing a plurality of FABSDAMs capable of binding to said FABSDB-Ls or a plurality of FABSDB-Ls capable of binding to said FABSDAMs in contact with said fluid; (c) allowing said FABSDAMs and said FABSDB-Ls to bind in a force-activated bond stress-dependent manner; and (d) detecting and quantitatively measuring the amount of binding of said FABSDAMs to said FABSDB-Ls; wherein said amount of binding is indicative of the rate of flow of said fluid. In the practice of this invention, the plurality of FABSDAMs or FABSDB-Ls placed in contact with said fluid can be bound to a substrate. The substrate can be a channel wall in contact with said fluid. The channel can be a microchannel. In the practice of this invention, the step of detecting and quantitatively measuring can include measuring light scattering of said fluid. Many methods are known in the art for detecting and quantitatively measuring the amount of binding of particles in a fluid and are useful in the practice of this invention.

This invention provides a method for delivering a particle to a surface of a system, said surface having attached thereto one member of an FABSDAM/FABSDB-L pair, said system also comprising a fluid in contact with said surface, said method comprising: (a) adding to said fluid the other member of said pair attached to said particle; and (b) allowing said pair members to bind in a force-activated bond stress-dependent manner.

In an embodiment of this invention, the surface is a surface of a deposit lining a blood vessel wherein said deposit constricts the flow of blood through said vessel. In an embodiment of this invention, the surface is a surface of a biomedical implant, a heart valve, or a stent.

In the practice of this invention, the system can also comprise a second surface in fluid contact with said first surface, wherein said second surface comprises said first member, wherein said members do not bind at said second surface. In an embodiment of this invention, a first shear stress is applied to said FABSDAM at said first surface and a second shear stress to said FABSDAM at said second surface wherein said first shear stress is between a lower force-activated shear-stress-dependent threshold of said FABSDAM and an upper force-activated shear stress-dependent threshold of said FABSDAM, and said second shear stress is less than said lower force-activated shear stress-dependent threshold or more than said upper force-activated shear stress-dependent threshold.

In an embodiment of this invention, the particle is a pharmaceutical. In an embodiment of this invention, the pharmaceutical is capable of removing a deposit lining the interior of a blood vessel. Pharmaceuticals useful for removing unwanted deposits lining the interiors of arteries are known in the art. In a clotted artery, at the clog, because the cross-sectional area of the channel opening is smaller, the blood flow rate is higher than at unclotted sections of the artery. Consequently, the bond stress applied to a FABSDAM in the clotted section is greater than the bond stress applied at unclotted sections of the artery. In an embodiment of this invention, FABSDAMs attached to pharmaceuticals capable of treating clotted arteries, do not adhere to FABSDB-Ls attached to the interior surface of the artery in unclotted sections, but do adhere to FABSDAMs attached to the interior surface of the artery and/or the interior surface of the clot in clotted sections.

This invention provides a bond stress-activated valve for controlling a fluid flow rate in a channel, said channel having a surface in contact with said fluid, said channel surface having attached thereto a plurality of a first member of an I-FABSDAM/FABSDB-L pair, said fluid comprising a plurality of the second member of said pair, wherein said first and second members are present in an amount sufficient to clog or partially clog said channel when bound in complexes in a force-activated bond stress-dependent manner. In the practice of this invention, the valve can be a microvalve, wherein said channel is a microchannel. In the practice of this invention, the fluid can have a first flow rate through said channel, wherein when said first flow rate changes a bond stress on said I-FABSDAMs, said change resulting in a binding strength change in the binding of said I-FABSDAMs and said FABSDB-Ls, thereby changing said flow rate.

This invention provides a bond stress-activated adhesive system comprising: (a) a plurality of I-FABSDAMs; and (b) a plurality of FABSDB-Ls capable of binding to said I-FABSDAMs in a bond stress dependent manner. In the practice of this invention, the I-FABSDAMs can be attached to a surface of a film. Methods are known in the art for attaching polypeptides to surfaces of films. In the practice of this invention, the FABSDB-Ls can also be attached to said film, whereby said film is capable of adhering in a force-activated bond stress-dependent manner to itself. In the practice of this invention, the FABSDB-Ls can be attached to a second film whereby said second film is capable of adhering in a force-activated bond stress-dependent manner to said first film.

This invention provides a method for making a bond stress-activated adhesive system comprising: (a) attaching a first member of an I-FABSDAM/FABSDB-L pair to a surface of a first film; and (b) attaching the second member of said pair to a surface of a second film. In an embodiment of this invention, the method also comprises (c) attaching said second member to said surface of said first film, and (d) attaching said first member to said surface of said second film. In an embodiment of this invention, said first film is attached to first object and the second film is attached to a second object whereby the first and second object may be bound in a force-activated bond stress-dependent manner.

This invention provides a viscosity modifier comprising a plurality of I-FABSDAMs and a plurality of FABSDB-Ls, said I-FABSDAMs and FABSDB-Ls being capable of binding to each other in force-activated bond stress-dependent manner.

This invention provides a method of modifying the viscosity of a fluid comprising: (a) adding to said fluid a plurality of I-FABSDAMs; (b) adding to said fluid a plurality of FABSDB-Ls capable of binding in a shear stress-dependent manner to said I-FABSDAMs; and (c) changing a bond stress on said I-FABSDAMs. In an embodiment of this invention, the I-FABSDAMs and FABSDB-Ls are attached to a plurality of objects.

This invention provides a method of interfering with the force-activated bond stress-dependent binding of a FABSDAM and a FABSDB-L capable of binding to said FABSDAM in a force-activated bond stress-dependent manner, said method comprising contacting said FABSDAM with an antibody capable of binding said FABSDAM but incapable of binding to a FABSDB-L-binding domain of said FABSDAM; and allowing said antibody to bind said FABSDAM. In the practice of this invention, FABSDAM can be a FimH polypeptide, wherein said antibody is capable of binding to a domain of said FimH polypeptide selected from the group consisting of FimH amino acids 25-31 (SEQ ID NO: 1), FimH amino acids 110-123 (SEQ ID NO: 2), and FimH amino acids 150-160 (SEQ ID NO: 3). This invention provides a method for interfering with the force-activated bond stress-dependent binding of a bacterium, comprising a FABSDAM, to a FABSDB-L, said method comprising contacting said FABSDAM with an antibody capable of binding said FABSDAM but incapable of binding to a FABSDB-L-binding domain of said FABSDAM; and allowing said antibody to bind said FABSDAM. Methods of making antibodies are known in the art.

This invention provides monoclonal and polyclonal antibodies generated using, and capable of binding to, a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3). This invention provides a polyclonal antibody generated using, and capable of binding to, a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3). This invention provides immunogenic compositions comprising a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3). The immunogenic polypeptides can be produced synthetically. Methods for isolating and synthesizing polypeptides are known in the art. In an embodiment of this invention, antibodies are generated using polypeptides having the sequence of SEQ ID NO:4 or SEQ ID NO:5. Monoclonal or polyclonal antibodies may be generated to the force-activated structure of a FABSDAM polypeptide, e.g., the FABSDAM bound to a FABSDB-L or a mutated FABSDAM polypeptide that naturally takes the conformation of a force-activated structure without a force having been applied. This structure may be different from the equilibrium structure of the FABSDAM. As is known to the art, antibodies may be produced using the bound FABSDAM/FABSDB-L pair.

This invention provides a method for making an engineered FimH polypeptide having different force-activated bond stress-dependent binding strength to a selected FABSDB-L than a natural FimH polypeptide, said method comprising engineering a DNA sequence encoding a FimH polypeptide to encode an engineered FimH polypeptide and expressing said engineered FimH polypeptide, wherein said engineered polypeptide comprises an amino acid substitution at an amino acid position selected from positions 154-156, position 32, and position 124. In the practice of this invention engineering can include engineering a codon selected from the group consisting of codons encoding valine at positions 154, 155, and 156 to encode proline, engineering the codon encoding glutamine at position 32 to encode a leucine, or engineering the codon encoding serine at position 124 to encode an alanine.

In an embodiment of this invention, the engineered FimH comprises a disrupted bond stress domain-stabilizing bond to a surrounding loop region, wherein said engineered FimH comprises a reduced force-activated bond stress-dependent lower threshold. In an embodiment of this invention, the engineered FimH comprises a bond stress dependent domain linker chain which is stabilized against extension. Information on the crystal structure of E. coli FimH can be found at www.pdb.org under accession number 1QUN. In an embodiment of this invention, the different force-activated bond stress-dependent binding comprises an increased force-activated bond stress-dependent lower threshold. In an embodiment of this invention, the engineered FimH has a disrupted hydrogen bond between linker-stabilizing loops 3 and 4 or between linker stabilizing loops 9 and 10. In an embodiment of this invention, the engineered FimH comprises one less hydrogen bond, relative to FimH-f18, between linker-stabilizing loops 3 and 4 or between linker stabilizing loops 9 and 10. In an embodiment of this invention, the engineered FimH comprises a force-activated bond stress-dependent domain linker chain which is stabilized against extension. In an embodiment of this invention, the engineered FimH comprises an increased force-activated bond stress-dependent lower threshold compared to FimH-f18.

This invention provides FimH polypeptides having an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, and SEQ ID NO:12.

This invention provides a method for changing binding strength of an isolated force-activated bond stress-dependent adhesion molecule (I-FABSDAM) to a force-activated bond stress-dependent binding ligand (FABSDB-L) for said I-FABSDAM, said method comprising changing a bond stress on said I-FABSDAM; wherein said binding strength increases when said bond stress decreases and decreases when said bond stress increases; wherein said bond stress is between an upper force-activated bond stress-dependent threshold of said I-FABSDAM and a higher force-activated bond stress-dependent binding threshold of said I-FABSDAM. In an embodiment of this invention, the higher binding threshold is a bond stress which is greater than said upper force-activated bond stress-dependent binding threshold and is a bond stress having the same binding strength as said lower force-activated bond stress threshold of said I-FABSDAM.

This invention provides a method for changing binding strength of an isolated force-activated bond stress-dependent adhesion molecule (I-FABSDAM) to a force-activated bond stress-dependent binding ligand (FABSDB-L) for said I-FABSDAM, said method comprising changing a bond stress on said I-FABSDAM; wherein said bond stress is higher than the lower force-activated bond stress threshold of said I-FABSDAM.

In the embodiments of this invention, force-activated bond stress such as shear can be created by many different mechanisms. These mechanisms include but are not limited to unidirectional flow, alternating fluid flow, circular flow, and turbulent flow, by sonication, by electromechanical devices or other mechanical actuators, or by dragging magnetic, charged or dielectric particles or beads that have been functionalized with adhesins or their respective ligands through the fluid, or mechanical impact.

### Definitions

As used herein, “force-activated bond stress-dependent adhesion molecule” and “FABSDAM” refer to molecules that are capable of binding ligands in a force-activated bond stress-dependent manner. FABSDAMs include, but are not limited to, adhesins, selectins, and integrins. Adhesion molecules include adhesins, selecting, integrins, cadherins, immunoglobulin superfamily cell adhesion molecules, and syndecans (Hauck C. R. (2002) Med Microbiol. Immuno. 191:55-62). FimH proteins are adhesins of bacterial origin. FimH polypeptides include all proteins that are structurally and functionally similar to bacterial derived FimH proteins, including, but not limited to all natural bacterial FimH variants, purified natural FimH proteins, engineered FimH polypeptides, mutated FimH polypeptides, chemically synthesized FimH polypeptides, and truncated but functional portions that are polypeptides of FimH proteins such as the lectin domain. FimH sequences can be found at GenBank Accession Nos. X05672 and AF288194. Methods for purifying FimH are known in the art (see Jones, 1995). As used herein, “isolated force-activated bond stress-dependent binding adhesion molecule” and “I-FABSDAM” refer to FABSDAMs that are not in the same context in which they exist in nature, including their natural in vivo context. All I-FABSDAMs are FABSDAMs. An E. coli that naturally has FimH-j96 protein, a naturally occurring variant of FimH, that has been transformed with an engineered FimH-f18 gene, isolated from a naturally occurring E. coli variant, and expresses FimH-f18, comprises two FABSDAMs but only one I-FABSDAM. Both FimH-j96 protein and FimH-f18 protein are FABSDAMs, but only the engineered and transformed FimH-f18 protein is an I-FABSDAM in this example. Even if the FimH-f18 (FimH-f18 is a natural strain) protein has the same sequence as the naturally occurring variant, because it is not in the in vivo context in which it is found in nature, it is isolated. Adhesins also include extracellular matrix adhesins, for example collagen adhesins of S. aureus which bind to collagen.

All methods and compositions described herein which use or comprise FABSDAMs may also use or comprise I-FABSDAMs.

As used herein, “force-activated bond stress-dependent binding ligand” and “FABSDB-L” refer to molecules that bind in a force-activated bond stress-dependent manner to FABSDAMs. FABSDB-Ls include molecules that also bond to other receptors which are not force-activated bond stress-dependent adhesion molecules. FABSDB-Ls that bind to bacterial adhesins include, but are not limited to, monomannose and trimannose. As used herein, “monomannose” refers to a single mannose molecule. A monomannose may be attached to another molecule, particle or substrate. As used herein, “trimannose” refers to three covalently bound mannose molecules. Trimannose may be attached to another molecule, particle or substrate. This also includes polypeptides derived from extracellular matrix proteins, including but not limited to fibronectin, collagen, laminin and osteopontin.

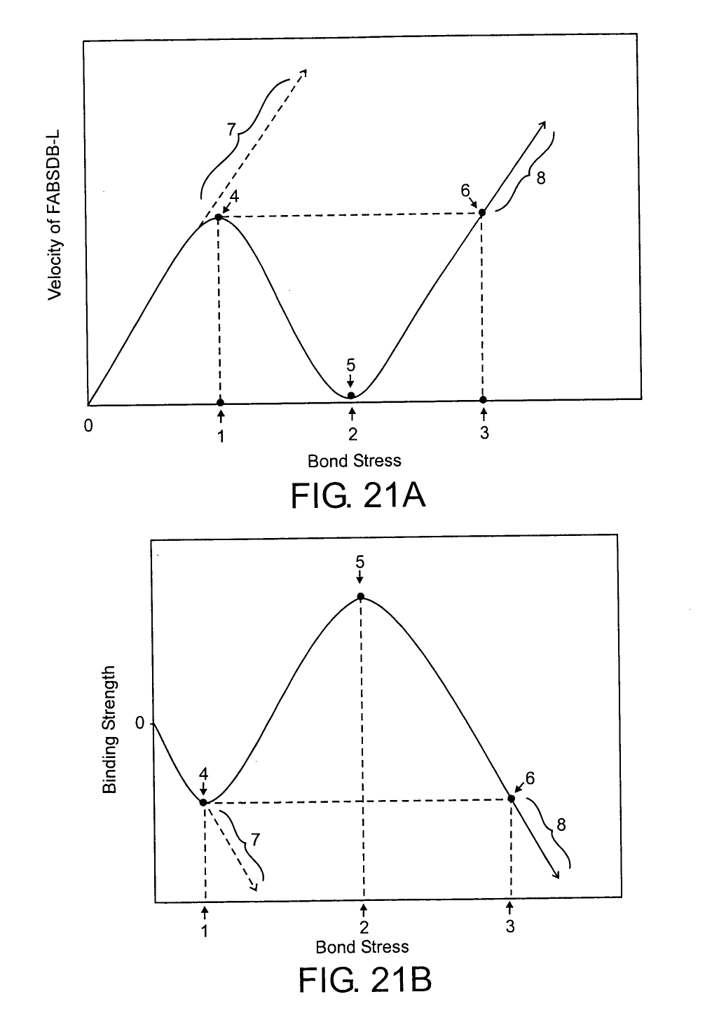

As used herein, “binding in a force-activated bond stress-dependent manner” refers to the ability of FABSDAMs to bind to FABSDB-Ls in a manner whereby the binding strength is dependent on the bond stress on the FABSDAM, wherein the bond stress on the FABSDAM is greater than the lowest bond stress at which as bond stress increases the binding strength increases (see FIGS. 21 and 22). When a FABSDAM and a FABSDB-L are capable of binding in a force-activated bond stress-dependent manner, within a range of bond stresses to be defined hereafter, the bond stress is positively correlated with binding strength. Within this range of bond stresses, as the bond stress on the FABSDAM increases, the binding strength of the FABSDAM to the FABSDB-L increases, and as the bond stress on the FABSDAM decreases, the binding strength of the FABSDAM to the FABSDB-L decreases. Binding strength changes can be continuous or stepwise. The range of bond stresses in which this occurs is bounded by a lower and upper threshold.

In a system comprising a given FABSDAM and FABSDB-L binding pair under specified conditions, there is a point at which increasing the bond stress on the FABSDAMs increases, rather than decreases the binding strength of the bonds between the FABSDAMs and FABSDB-Ls. This is called the “lower threshold.” When a small bond stress (below the lower threshold) is applied to a FABSDAM that is capable of binding to a FABSDB-L in a force activated bond stress-dependent manner, as is typically expected, if the two molecules are not bound to each other, they are less likely to bind, and if they are bound to each other, the bond strength between them is weakened. As the bond stress is increased, the bond stress reaches a “lower force-activated bond stress-dependent binding threshold” (also referred to as a “lower threshold) which is identified by a minimum point in a graph of binding strength versus bond stress (see FIG. 21). This lower threshold point is the point at which increasing bond stress on a FABSDAM begins to increase the binding strength with which it binds to a corresponding FABSDB-L. As the bond stress increases above the lower force-activated bond stress-dependent threshold, the binding strength of the FABSDAM to the FABSDB-L increases with increasing bond stress. As the bond stress is increased, the bond stress finally reaches an “upper force-activated bond stress-dependent binding threshold” (also referred to as an “upper threshold”) which is identified by a maximum point on the graph (FIG. 21). As used herein, the “upper force-activated bond stress-dependent binding threshold” (upper threshold) refers to the bond stress at which this maximum occurs. As the bond stress increases above the upper force-activated bond stress-dependent binding threshold, the binding strength of the FABSDAM to the FABSDB-L decreases, as is typically expected, however, the binding strength is still greater than it would be at bond stresses above the upper threshold if the FABSDAM and FABSDB-L were not able to bind in a force-activated bond stress-dependent binding manner (as can be predicted by extrapolating from the portion of the graph at bond stresses below the lower force-activated bond stress-dependent threshold). The amounts of force required to reach the lower and upper thresholds are specific to each ligand-bound FABSDAM. The lower and upper bond stress thresholds are specific to each FABSDAM.

As used herein, “bond stress” refers to a force which tends to pull a bonded FABSDAM and FABSDB-L apart. It may be a shear force, a tensile force, or any combination thereof. Stress is known in the art as force divided by area. A force that applies shear stress is a force that is parallel to a plane on which it acts. This plane can be the surface of a fluidic device. Forces can have shear and/or tensile components. A shear stress applied to a FABSDAM consists of the forces that are parallel to the binding plane of an FABSDAM and a SDDB-L bound to it. The binding axis of the FABSDAM is the axis through only one point of the binding plane and perpendicular to the binding plane. The binding axis also projects through the FABSDAM and its bound FABSDB-L. The forces that contribute to a shear stress are therefore also perpendicular to the binding axis of the FABSDAM and its bound FABSDB-L (or perpendicular to the eventual binding axis if the FABSDAM and the FABSDB-L are not bound). Note that the shear stress given in the figures is given with respect to the surface of the fluidic device. As used herein, “tensile force” refers to forces along the binding axis that are opposite to the direction of the binding force. As used herein, “applying a shear stress to a FABSDAM” refers to applying a force per area that is perpendicular to the binding axis of the FABSDAM. Note that after the force is applied, the FABSDAM may reorient so that the force is no longer perpendicular to the binding axis. As used herein, “applying a tensile force to a FABSDAM refers to applying tensile forces parallel the binding axis of the FABSDAM and its bound FABSDB-L (or parallel to the eventual binding axis if the FABSDAM and the FABSDB-L are not bound) which forces are opposite to the binding force and tend to pull the FABSDAM and FABSDB-L apart, and are applied over part or all of the binding plane between the FABSDAM and its bound FABSDB-L. Tensile forces can be generated from shear forces or can be generated by other means such as by gravitational or magnetic forces. When a shear stress is applied to a FABSDAM, the FABSDAM may reorient relative to the shear stress such that a tensile force is being applied to the FABSDAM. The FABSDAM may reorient such that all of the shear forces become tensile forces. As used herein, “changing a bond stress” refers to increasing or decreasing a bond stress. Shear forces and tensile forces may be applied to a FABSDAM directly or indirectly. Indirect tensile forces may be applied by shear forces. Indirect forces may also be applied through a FABSDB-L or particles or substrates attached to the FABSDAM or FABSDB-L.

Binding kinetics and bond strength of a receptor and a ligand, such as a FASDAM and a FABSDB-L, can be described using on-rate and off-rate (http://www.med.unc.edu/wrkunits/2depts/pharm/receptor/lesson1.htm). Binding of a receptor and ligand occurs when the ligand and receptor collide (due to diffusion) in an orientation that leads to a binding event. The on-rate (number of binding events per unit of time) equals [Ligand]*[Receptor]*kₒₙ. The off-rate (number of dissociation events per unit time) between a receptor and a ligand equals [ligand*receptor]*k_{off}. The probability of dissociation is the same at every instant of time. The receptor doesn't “know” how long it has been bound to the ligand. After dissociation, the ligand and receptor are the same as at they were before binding. If either the ligand or receptor is chemically modified, then the binding does not follow the law of mass action.

As used herein, “FABSDAM/FABSDB-L pair” refers to a FABSDAM and a FABSDB-L that are capable of binding in a force-activated bond stress-dependent binding manner. “FABSDAM/FABSDB-L pair” refers to the identities of a set of a FABSDAM and a FABSDB-L, but does not imply actual molecules, numbers of molecules, or whether individual molecules that are examples of a pair are bound or unbound.

FABSDAMs are capable of being bound to FABSDB-Ls in two states. As used herein, “tight binding” and “tightly bound” (also referred to as “catch binding” or “firm binding”) refers to a FABSDB-L and a FABSDAM in a state of high binding strength such that they do not become substantially unbound (disassociated) under the conditions of the system they are in. As used herein, “rolling” or “weak” (also called “slip”) binding refer to a FABSDB-L that is loosely or transiently bound to a FABSDAM wherein the FABSDB-L and the FABSDAM are in a state of low binding strength, where they may easily come unbound and rebind to each other. As used herein, “bound” refers to both tight binding and rolling (weak) binding. If weak binding dominates, particles with either FABSDAMs or the FABSDB-Ls attached to their surface either transiently adhere or roll over fixed surfaces to which the complements FABSDB-Ls or FABSDAMs are attached. As used herein, “unbound” refers to neither tight nor rolling binding but to not being bound at all. As used herein, “changing binding strength” refers to changing the quantity of binding strength of a FABSDAM/SDB-L pair. Binding strength may be quantitated for a plurality of FABSDAMs and FABSDB-Ls by time-lapse photography. If either the FABSDAMs or the FABSDB-Ls are in a fixed position and the particle-attached complements FABSDB-Ls or FABSDAMs, respectively, float freely in a fluid which is in contact with the fixed FABSDAMs or FABSDB-Ls, the number of particles that stay in a fixed position over time can be counted, as can the number of particles that roll various distances over time. The ratio of particles at different binding strengths may be counted over a selected time period for a selected area and density of FABSDAMs and/or FABSDB-Ls. When changing binding strength comprises increasing binding strength, the ratio of particles that are tightly bound to those that are loosely bound increases. When changing binding strength comprises decreasing binding strength, the ratio of particles that are tightly bound to those that are loosely bound decreases. Binding strength may also be assessed using time-lapse photography when the FABSDB-Ls are in fixed positions and the FABSDAMs are floating. As used herein, “binding strength increases” refers to an increasing ratio of tightly bound to rolling FABSDAMs or FABSDB-Ls attached to their surfaces. As used herein, “binding strength decreases” refers to a decreasing ratio of tightly bound to rolling FABSDAMs or FABSDB-Ls.

The term “polypeptide” as used herein includes proteins. As used herein, “adhesin” refers to a family of lectin proteins used by bacteria to adhere to host cells. In bacteria, adhesins are normally located on pili or fimbriae which are thin, proteinaceous organelles that extend from the surface of many gram-negative bacteria. Adhesins bind specific carbohydrates. As used herein, “FimH” is an adhesin normally found at the tip of type I pili in most enterobacteria, including many E. coli strains. As used herein, “E. coli FimH protein” refers to a FimH protein that is naturally occurring in E. coli. A sequence of an E. coli FimH protein can be found at Genbank Accession Number P08191. As used herein, “FimH-f18 protein” refers to the FimH protein naturally occurring in E. coli strain F 18. As used herein, “FimH-j96 protein” refers to the FimH protein naturally occurring in E. coli strain J96. Polypeptides corresponding to the above proteins may be full-length or truncated polypeptides having all or a portion of the amino acid sequences of the corresponding proteins.

As used herein, “selectin” refers to proteins used by leukocytes to transiently adhere to blood vessel walls (http://hsc.virginia.edu/medicine/basic-sci/biomed/ley/selectins.htm). Selectins are a family of transmembrane molecules, expressed on the surface of leukocytes and activated endothelial cells. Selectins contain an N-terminal extracellular domain with structural homology to calcium-dependent lectins. The initial attachment of leukocytes, during inflammation, from the blood stream is afforded by the selectin family, and causes the leukocyte velocity to decrease. This rolling is mediated by a slow downstream movement of leukocytes along the endothelium by transient, reversible, selectin interactions. Each of the three selectins can mediate leukocyte rolling given the appropriate conditions. L-selectin is the smallest of the vascular selectins, and can be found on most leukocytes. P-selectin, the largest selectin, is expressed primarily on activated platelets and endothelial cells. E-selectin is expressed on activated endothelium with chemically- or cytokine-induced inflammation. Von Willebrand factor (VMF) interacts with members of the FABSDAM/FABSDB-L family. VMF undergoes a conformational change that allows flowing platelets to reversibly bind to a surface by way of their GP Ib complex. This binding is followed by stable platelet adhesion (integrin α_{IIb}β₃) to a haemostatic surface as provided by collagen or fibrin fibers (Keurin et al. (May 2003) J. Lab. Clin. Med. 141(5):350-358). P-selectins bind to mucin.

As used herein, “isolated molecule” refers to a molecule that has been purified from a context in which it is found in nature or is otherwise no longer in the context in which it is found in nature. As used herein, “synthetic molecule” refers to a molecule which is chemically synthesized. As used herein, “prokaryotic cell” refers to a cell of a prokaryotic organism as known in the art, including a bacterium. As used herein, “eukaryotic cell” refers to a cell of a eukaryotic organism as known in the art, including mammalian cells. As used herein, “organism” refers to a whole living being, e.g., a bacterium. As used herein, “synthetic substrate surface” refers to a surface or a portion of a surface of a supporting material that is not natural.

As used herein, “N/cm²” refers to Newtons per centimeter squared, as units for stress. As used herein, “dynes/cm²” and “d/cm²” refer to dynes per centimeter squared, as units for stress. As used herein, “pN/μm²” refers to picoNewtons per micrometer squared, as units for stress.

As used herein, “attached” refers to being connected, e.g., covalently bonded, non-covalently bonded, cross-linked, embedded, adhered, directly connected, and indirectly connected. Indirect connection may include the use of a linker.

As used herein, “capable of being bound” refers to a component that has the capacity and ability to be bound to another component. If a component is described as capable of being bound, neither the component nor anything to which it is attached interferes with the capacity and ability of the component to bind.

As used herein, “substantially uniform material” refers to a material wherein any randomly selected portion of the volume of the material has the same composition and properties as any other portion, when the volume contains at least several multiples of the number of components used to form the material

As used herein, “cross-linking” refers to forming covalent bond links between two or more components.

As used herein, “selected surface” refers to a surface area chosen in preference to another surface area, wherein a surface is an exterior boundary of an object. A selected surface can be an entire surface. As used herein, “selected three-dimensional form” refers to a form chosen in preference to another form, wherein a three-dimensional form is the three-dimensional shape of a volume. A “plurality of selected three-dimensional forms” as used herein refers to a plurality of three-dimensional objects all having the same shape and size. As used herein, “layer” refers to a material that is organized in a form such that one dimension approaches zero or is small compared to the other two dimensions of a three-dimensional form.

As used herein, “chain” refers to a series of objects connected one to another in a series. As used herein, “directional chain composed of cylinders” refers to a series of cylindrical objects that are not symmetric along the cylindrical axis which are connected to one another in a series wherein each member of the series is oriented in the same direction as every other member. As used herein, “alternating link chain” refers to a chain composed of two different selected three-dimensional forms, e.g., cubes and spheres, alternating with each other.

As used herein, “clog” refers to partially or completely hindering or obstructing flow of a fluid. As used herein, “sufficient” refers to an amount at least adequate for a purpose. If an amount of an object is sufficient to clog a device through which fluid is flowing, the amount of the object is sufficient to detectably slow the flow of the fluid and could be enough to completely stop the flow of the fluid or is sufficient to detectably increase the pressure drop, wherein the pressure drop is the pressure downstream of the clog subtracted from the pressure upstream of the clog. A change in bond stress sufficient to cause release of a first particle attached to FABSDB-Ls from a fixed surface to which FABSDAMs are attached, but insufficient to cause release of a second particle attached to FABSDB-Ls from a fixed surface to which FABSDAMs are attached, can be determined by one skilled in the art without undue experimentation by testing the system components under different bond stress conditions. Similarly, a change in bond stress sufficient to cause binding of a first FABSDB-L to a FABSDAM but insufficient to cause binding of a second FABSDB-L to the same FABSDAM can be determined by one skilled in the art without undue experimentation by testing the system components under different bond stress conditions.

As used herein, “channel” refers to a structure minimally comprising one or more bottom walls and side walls, and optionally comprising one or more top walls, and defines a space through which a fluid may be directed. Walls may be horizontal, or vertical, above or below, including floors and ceilings. A channel can comprise a continuous cylindrical wall without corners, such as a glass tube or a blood vessel.

As used herein, “recirculating channel” refers to a channel through which an object can move and pass back to its starting point. In this invention, a recirculating channel having a fluid flow through it wherein the fluid contains FABSDAMs and/or FABSDB-Ls allows the FABSDAMs and/or FABSDB-Ls to be recirculated so that they do not have to be replenished. As used herein, “exit port” refers to an opening in a channel through which an object or fluid can exit from a channel. As used herein, “exit channel” refers to a channel connected to the exit port of another channel. As used herein, “bypass port” refers to an opening in a channel other than an exit port through which an object or fluid can exit the channel. As used herein, “bypass channel” refers to a channel connected to the bypass port of another channel.

As used herein, a “fluidic device” is a device comprising means for fluid flow such as channels, baffles, walls, ports, chambers, and the like. A microfluidic device is a device comprising components having at least one dimension less than 5 mm, and preferably less than 1 mm.

As used herein, “target particle” refers to a particle that is a target of an action. As used herein, “target particle binding agent” refers to an agent capable of binding to a target particle, e.g., an antibody to the target particle. As used herein, “removing agent” refers to an agent useful for removing an object or sequestering an object, e.g., a magnetic bead or an antibody. As used herein, “removing” refers to taking an object from one context and placing it into another local context. Removing includes separating, sequestering, isolating, and purifying.

As used herein, a “removing force” is a force applied to complexes hereof to remove them from one context to another. Such “removing forces” include the force of gravity, fluid pressure, magnetic force and electrical force, and other forces known to the art as used in separation processes. As used herein, “sedimentation” refers to the process of utilizing the mass of an object to remove it. As used herein, “filtration” refers to passing a fluid through a filter, wherein at least one object in the fluid does not also pass. As used herein, “bioseparation” refers to a method of using biologically derived materials or materials imitating biological materials to separate objects, e.g., antibody precipitation.

As used herein with respect to two FABSDB-Ls, their capacity to “induce different bond stresses” refers to the ability of the two FABSDB-Ls in a common environment to confer different bond stresses on a FABSDAM bound to them. The two different FABSDB-Ls may differ in characteristics such as surface area, diameter, texture, mass, magnetic and/or electrical properties which will affect the bond stress placed on a FABSDAM bound to them. As used herein, “same conditions” refers to such a common environment.

As used herein with respect to a bond stress, “insufficient to cause binding” refers to the bond stress being incapable of causing tight binding or rolling (transient binding) in a selected environment.

As used herein, “selective binding” refers to binding of selected objects to the exclusion of other objects. When a FABSDB-L is selectively bound, another object that is not bound could be a different FABSDB-L, if present. Selective binding and releasing means that although something else is capable of being bound, due to the context (the system conditions), it is not bound. As used herein, “selectively releasing” refers to releasing of selected bound objects to the exclusion of different bound objects. As used herein, “release” refers to reduction of the ratio of tightly bound FABSDAM/FABSDB-L pairs to rolling FABSDAM/FABSDB-L bound pairs and/or unbound pairs, and includes the state wherein no FABSDB-Ls are tightly bound, the state wherein no FABSDB-Ls are rolling, and the state where all FABSDB-Ls are unbound.

As used herein, “detecting and quantitatively measuring an amount of binding” refers to qualitatively measuring binding and, if binding is present, also quantitatively measuring the amount of binding or the strength of binding. In the practice of this invention, an amount of binding of FABSDAMs and FABSDB-Ls in a transparent fluid may be measured by passing light through the fluid and measuring the light scattering of the light by the fluid. Light scattering is known in the art as light waves propagating in a material medium, wherein the direction, frequency, or polarization of the wave is changed when the wave encounters discontinuities in the medium, or interacts with the material at an atomic or molecular level. The amount of binding of the FABSDAMs and FABSDB-Ls affects the light scattering. This measuring method may be calibrated before making measurements of unknown samples; or measurements can be made in a comparative manner by changing the binding stress on the sample and measuring repeatedly to determine the value and the extent of change in the amount of binding caused by changes in the binding stress. As used herein, “amount of binding is indicative of the rate of flow” refers to a system in which the binding strength of FABSDAM/FABSD-L pairs is changed by changes in the rate of flow of the fluid containing them, such that there is a correlation between the amount of binding and the rate of flow of the fluid.

As used herein, “microchannel” refers to a channel that is microscopic in size, i.e., having at least one dimension of less than 5 mm. Microchannels may be designed to enable laminar flow of fluids in preference to turbulent flow of fluids.

As used herein, “bond stress-activated adhesive system” refers to a system for adhering objects wherein the strength of adherence is increased with increasing bond stress and decreased with decreasing bond stress. A bond stress-activated adhesive system includes force-activated bond stress dependent binders, I-FABSDAMs and FABSDB-Ls, as well as means for attaching the binders to objects to be adhered by bond stress. Such means may include chemical moieties such as biotin-avidin pairs, antibody-antigen pairs and the like. These means may include adhering components that are not force-activated bond stress-dependent. Bond stress-activated adhesives and bond-stress activated adhesive systems are a subset of pressure-sensitive adhesives. Pressure-sensitive adhesives are useful in fields ranging from semiconductor manufacturing to construction. Pressure sensitive adhesive systems are useful, for example, as diaper closure tapes as well as other tapes, labels, and films.

As used herein, “immunogenic composition” refers to a composition useful for giving rise to antibodies by methods known in the art for making monoclonal or polyclonal antibodies. Monoclonal antibodies useful in this invention are obtained by well-known hybridoma methods (Harlow and Lane (1988) Antibodies: A Laboratory Manual, Cold Spring Harbor Laboratories; Goding (1986) Monoclonal Antibodies: Principles and Practice, 2d ed., Academic Press, New York; and Ausubel et al. (1993) Current Protocols in Molecular Biology, Wiley Interscience/Greene Publishing, New York, N.Y.). Methods for making polyclonal antibodies are well known in the art.

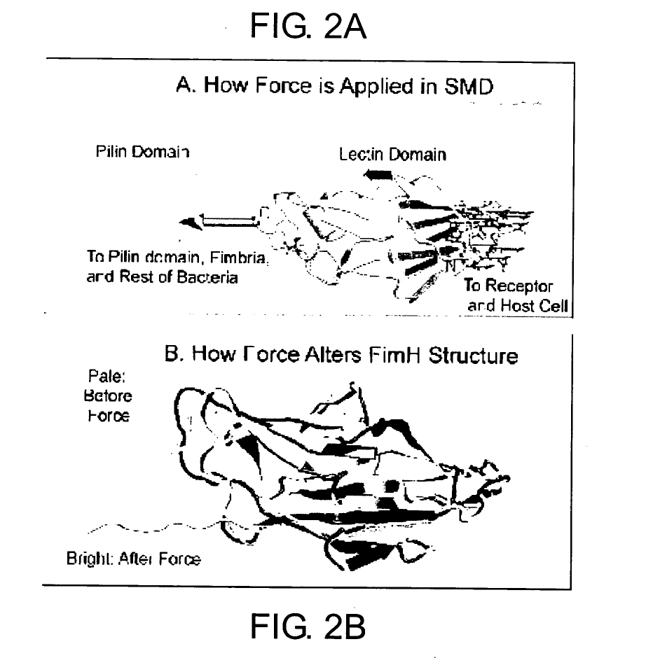

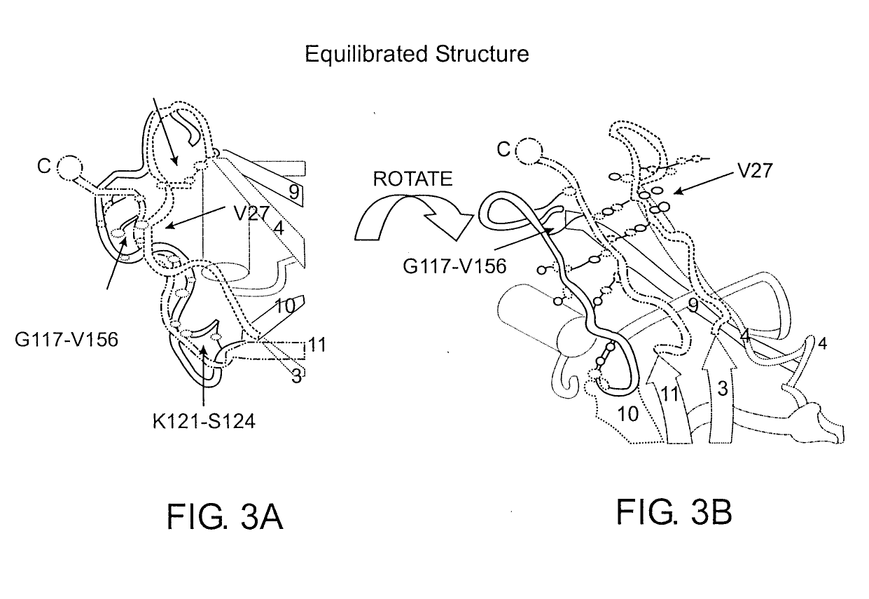

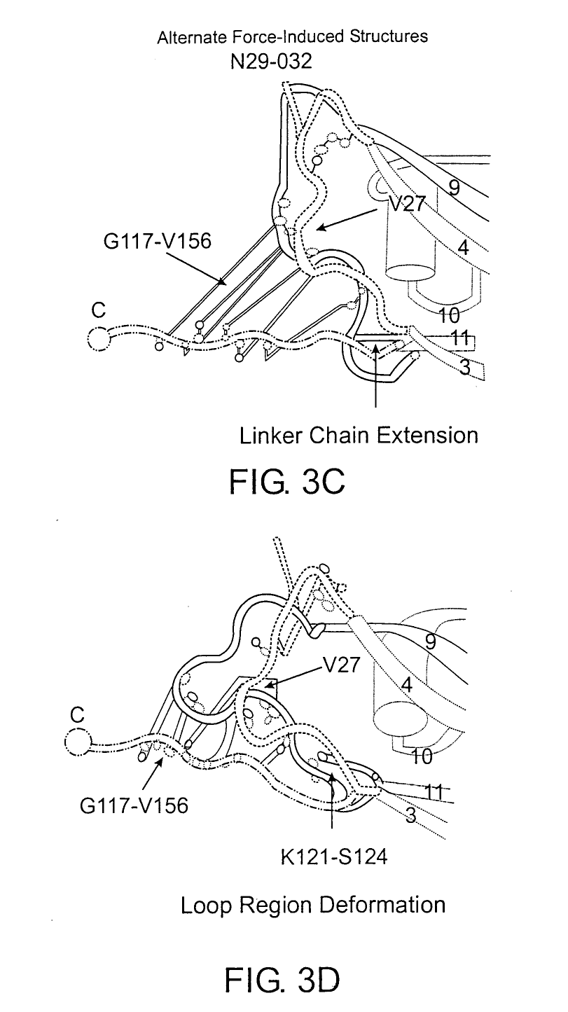

As used herein, “bond stress-stabilizing bond to a surrounding loop region” refers to stabilizing hydrogen or sulfide bonds that form between portions of a FABSDAM such as described below and make the FABSDAM capable of forming tighter bonds with FABSDB-Ls to which they bond than the same molecules which lack such stabilizing bonds. The valines at amino acid positions of the lectin domain of an FimH FABSDAM that form with amino acids GVAI at positions 117-120 of the 9-10 loop and amino acids PVV at positions 26-28 in the 3-4 loop (See FIGS. 2 and 3) are examples of stabilizing bonds. These bonds are broken by increasing bond stress on the FABSDAM which increases the binding strength of the FABSDAM to a FABSDB-L. As used herein, “disrupted bond” refers to a bond that is broken or prevented from forming. The bonds may be disrupted by methods known in the art, such as by removing the proton donors and acceptors by changing the amino acids at the locations involved in bonding.

As used herein, “bond stress-dependent linker chain stabilized against extension” refers to a linker chain of a FABSDAM that has been modified to include additional bonds that must be broken by bond stress to increase bonding strength, or that has been modified to exclude bonds that stabilize extension of the linker, when extension leads to an increase in bond strength.

A viscosity modifier is a compound or a set of compounds that is capable of modifying the viscosity of a fluid. The viscosity modifiers of this invention are force-activated bond stress-dependent.

As used herein, “bound in complexes” refers to FABSDAMs and FABSDB-Ls that are bound in groups of more than one pair. If a plurality of FAIBSDAMs and FABSDB-Ls are attached to a plurality of objects, when the FABSDAMs and the FABSDB-Ls bind, they bind from one object to another. More than two objects bound by FABSDAMs and FABSDB-Ls are bound in a complex.

The term “particle” includes bacterial pili, isolated molecules, synthetic molecules, proteins, polypeptides, organelles, prokaryotic cells, eukaryotic cells, viruses, organisms, nanoparticles and microparticles, as well as other particles known to the art including pollutant particles, toxin particles and drug particles. The term “surface includes cell membranes, device surfaces, synthetic substrate surfaces, and other surfaces known to the art. The term “substrate” includes any particle or surface known to the art to which FABSDAMs and/or FABSDB-Ls can be attached.

As used herein, “interfering with force-activated bond stress-depending binding” refers to changing force-activated bond stress-dependent binding in a way that decreases the ability of a FABSDAM to bond to a FABSDB-L in a force-activated bond stress-dependent manner.

As used herein, a “surface of a system” is a surface of a particle, a device, a living organism, an organ or organelle, e.g., the interior or lumen of a blood vessel or, any other system known to the art. The “surface of a system” can be the entire surface of all components of the system, or can be all or part of a surface of one or more selected components of the system.

### DESCRIPTION OF THE DRAWINGS

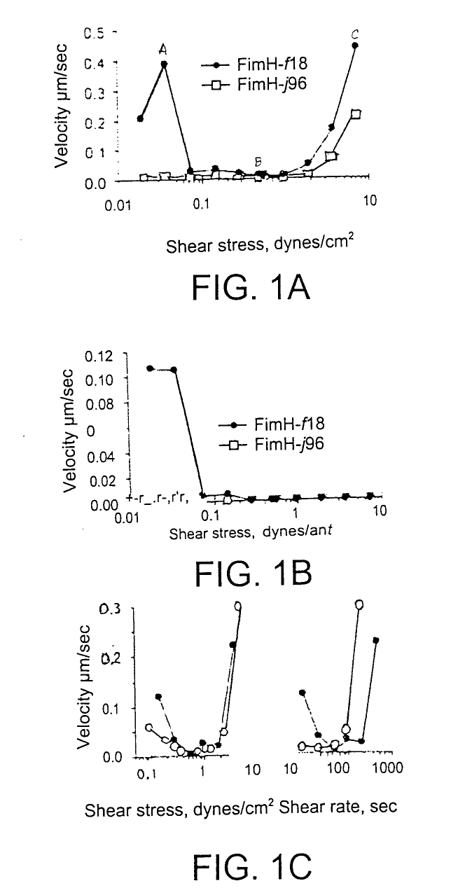

### FIGS. 1A-D. Movement of RBCs Bound to a Carpet of E. coli Under Shear in a Glycotech Parallel Plate Flow Chamber

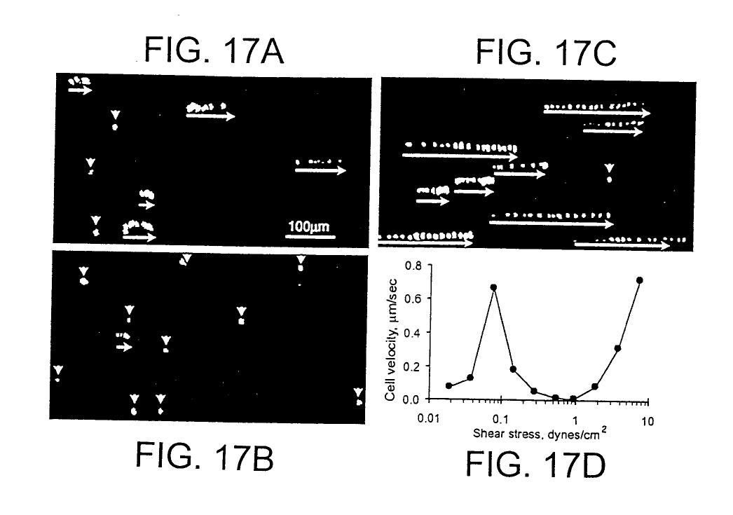

The movement shown in FIGS. 17A-C at each shear was analyzed as described in the Experimental Procedures and expressed as the average cell velocity, as shown in FIG. 1A for the low-Man1 binding FimH-f18 (●) and the high-Man1 binding FimH-j96 (□). Letters in FIG. 1A indicate the shear stress values corresponding to the images in FIGS. 17A-C. Cells move the most at low or high shear stress, while cells at intermediate shear stress (0.5 dynes/cm²) move very little. In addition to moving along the surface, some cells detached completely and moved at the fluid velocity. The rate of detachment is shown in FIG. 1B and was measurable only at low shear as cells rarely if ever detached at moderate and high shear. (FIGS. 1C-D): Effect of viscosity on the velocity of RBCs bound to a carpet of E. coli. In flow chamber experiments, RBCs were bound to E. coli expressing FimH-f18 and subjected to various shears. Buffers of two different viscosities were used in order to determine whether the shear stress or shear rate was the critical determinant for increasing binding under moderate shear. The solution was calculated to have a viscosity of 1.0 centipoise (●), while addition of 6% Ficoll increased the viscosity to 2.6 centipoise (●). (IC) When average cell velocities in the two conditions were plotted against shear stress, their drop to a minimum coincided. (1D) However, when the velocities were plotted against the shear rate, the curves did not coincide. This indicates that shear stress and the force on cells, rather than shear rate and kinetic effects, mediates the effects of fluid shear on adhesion.

### FIGS. 2A-B. Steered Molecular Dynamics (SMD)

FIG. 2A shows how force is applied to the structure of FimH-j96(Choudhury et al., 1999) hydrated in explicit water molecules (Thomas et al, 2002). FimH consists of two domains, the pilin domain (pale gold, left) and lectin domain (blue, right). The pilin domain integrates FimH into the tip of the pilus and through it to the rest of the bacteria. It binds to and was cocrystallized with the FimC chaperone protein in the published crystal structure (Choudhury et al., 1999). The lectin domain binds the receptor and is the only structure included in the SMD simulations. The N terminus (residue F1) and C terminus (residue T158) of this domain are indicated by the letters N and C. The residues that bind the nonphysiological receptor analog in the crystal structure are shown in green ball-and-stick (residues F1, 113, N46, D47, Y48, 152, D54, Q133, N135, Y137, N138, D140, and D141). In the SMD simulations, these 13 residues are pulled with equal force in one direction (small gold arrows) while the C-α carbon of residue T158 is pulled with the same net force in the opposite direction (large reddish gold arrow). The A27V mutation that is responsible for the increase in Man1 binding in FimH-j96 relative to FimH-f18 is shown in blue ball-and-stick (Sokurenko et al., 1995, 1998).

FIG. 2B Comparison of the structure of the FimH lectin domain before blue (light) and after blue (dark) force is applied. The two structures were aligned to show the RMSD of the β strands before and after a force has been applied. Large changes are observed in the C-terminal β-strand (yellow) that links the FimH lectin domain to the pilin domain. This same β-strand is bound via backbone hydrogen bonds to the adjoining loop regions (red and blue). However, the remainder of the protein (light blue) shows only small changes, including in the receptor-binding region (green). These figures were made using VMD, which was developed by the Theoretical Biophysics Group in the Beckman Institute for Advanced Science and Technology at the University of Illinois at Urbana-Champaign (Humphrey et al., 1996).

### FIGS. 3A-D. Structural Changes Occurring in the Interdomain Region of the FimH Lectin Domain During SMD Simulations

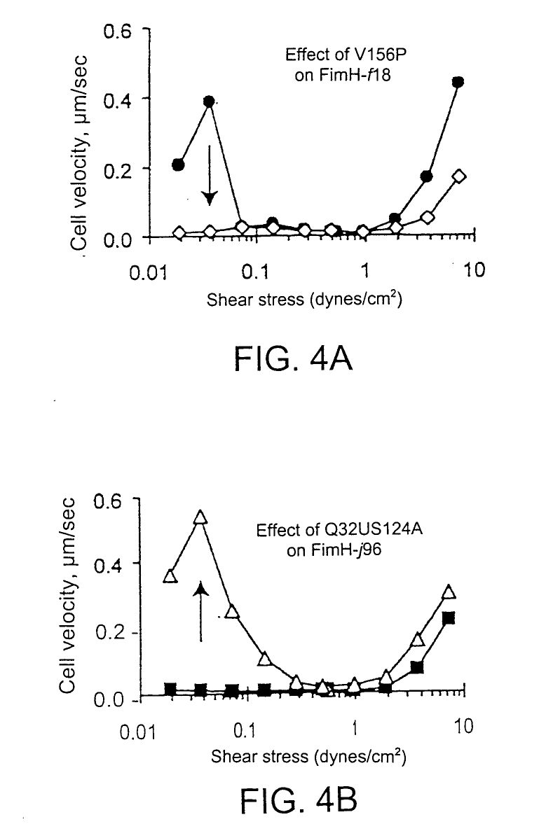

FIG. 3A: The equilibrated structure from the viewpoint used in FIG. 3. The linker chain (residues A150 to T158) is shown outlined in dash/dot (••--••--), the 3-4 loop is shown outlined in dashes (-----), and the 9-10 loop is shown outlined in a solid line. Color images with more detail are available in Thomas et al. (2002), FIG. 4. Loops are identified by the β-strands that they connect, and the residue and strand numbers reflect the terminology published with the crystal structure (Choudhury et al., 1999). Six hydrogen bonds that anchor the linker chain to the 3-4 and 9-10 loops in the crystal structure are shown as dash/dot ••--••-- lines. A hydrogen bond between the backbone hydrogen of residue N29 and the side chain carboxyl oxygen of Q32 is shown as a ----- dashed line. A hydrogen bond between the backbone oxygen of residue K121 and the side chain hydroxyl hydrogen of S124 is shown as a solid line The residues involved in these hydrogen bonds are shown in ball-and-stick representation, showing only the backbone atoms when the side chains are not involved in the bonds, to keep the figure cleaner. Residue V27 is shown in ball and stick, and residue T128 is shown as a dot/dash ball at the end of the linker chain. What appears in the Thomas paper as green is shown outlined in a dotted (•••••) line.

FIG. 3B: Lateral-to-front rotation of the equilibrated structure shown in FIG. 3A offers an alternative view of the six bonds to the linker chain (FIG. 3C). One pathway that was observed to occur upon application of force was linker chain extension. Shown here is a typical conformation resulting from linker chain extension, from the same viewpoint as in FIG. 3A. (FIG. 3D) In some simulations, an alternative pathway was observed in which the N29-Q32 is shown by a dashed line arrow and/or K121-S124 (shown by a solid line arrow) side chain hydrogen bonds broke, the 3-4 and 9-10 loops distorted, and the linker chain separated more slowly from the loop regions if at all. Shown here is a typical conformation resulting from loop region deformation, from the same viewpoint as in FIG. 3A. These figures were made using VMD, which was developed by the Theoretical Biophysics Group in the Beckman Institute for Advanced Science and Technology at the University of Illinois at Urbana-Champaign (Humphrey et al., 1996).

### FIGS. 4A-B.

Effect of FimH Mutations on the Velocity of Red Blood Cells (RBCs) Bound to a Carpet of E. coli. Reduction of the velocity of surface bound RBCs reflects enhanced adhesion. (FIG. 4A) In flow chamber experiments, RBCs on bacteria expressing FimH-f18 with the V1 56P mutation in the linker chain (⋄) moved less under low shear stress than did those on FimH-f18 (●). Thus, as predicted by SMD, this mutation decreased the amount of force needed to increase adhesion due to a partial destabilization of the linker chain of FimH-f18. (FIG. 4B) RBCs on bacteria expressing FimH-j96 with the Q32L/S124A mutations in the loop regions near the linker chain (Δ) moved much more under low shear stress than those on FimH-j96 (▪). This is consistent with the SMD prediction that this mutation would increase the force needed to activate adhesion, due to a partial stabilization of the linker chain of FimH-j96. Experiments were performed and analyzed as in FIG. 2.

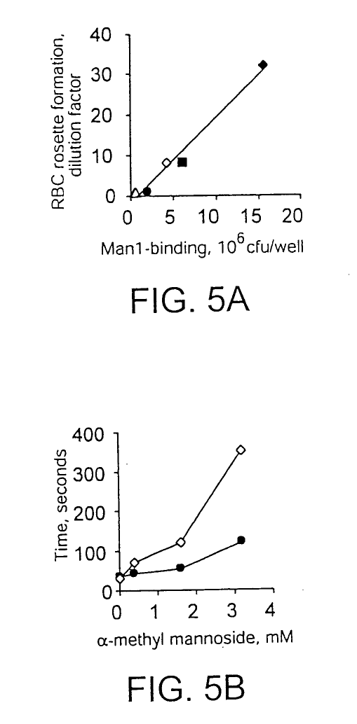

### FIGS. 5A-B.

Functional Significance of Shear Activation (FIG. 5A) Correlation between the ability of recombinant E. coli strains to agglutinate RBCs in static conditions and to bind Man1 receptors (see Table 1). (FIG. 5B) Effect of α-methyl-mannoside on the aggregation of RBC by E. coli bacteria expressing either FimH-f18 variant (●) or FimH-f18-V156P mutant (⋄) under dynamic conditions as described in Table 1.

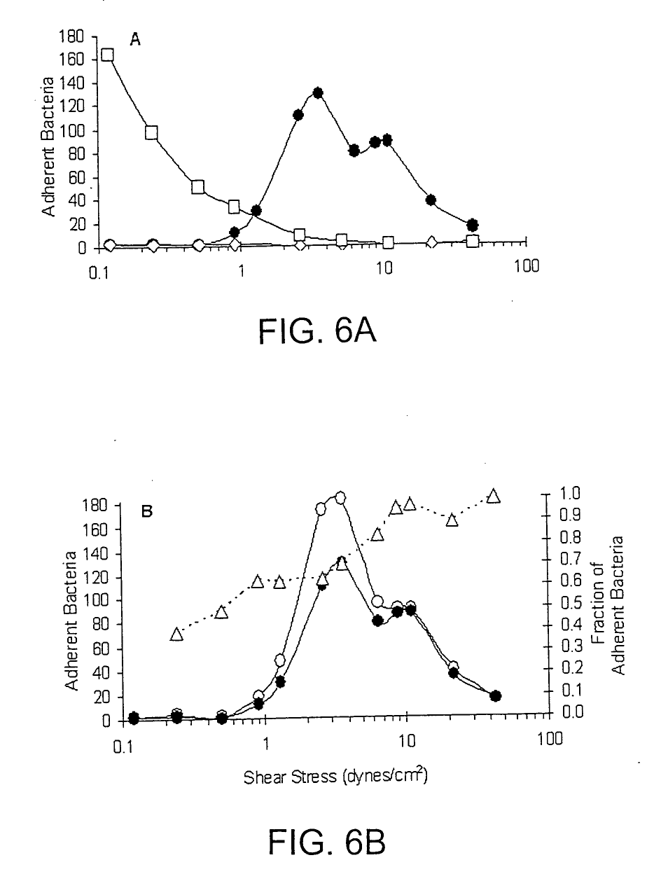

### FIGS. 6A-B

Accumulation of E. coli on purified receptors. (FIG. 6A) The accumulation of E. coli was measured over a range of shear stress on tissue culture dishes containing either the FimH ligand 1Man-BSA (closed circles ), which shows shear-activation, the negative control galactosylated BSA (open diamonds ⋄ galactose is not specifically recognized by FimH), or a polyclonal antibody to FimH (open squares □) which shows the classical “slip-bond” behavior where accumulation is reduced with shear. Accumulation on the surface was measured after 5.1 minutes of exposure to bacteria, using a 2 second shutter speed to blur out all free-floating cells at all shear rates. (FIG. 6B) In order to analyze the fraction of cells rolling on the surface, the same experiment was repeated using two shutter speeds of either 750 ms at 0.12 dynes/cm² or 2 ms at 19 dynes/cm² (open circles ∘). Two curves are given spanning the full shear stress range. The time intervals where chosen such that the free floating cells moved 10 mm while the shutter remained open. The fraction of bound bacteria that were stationary for 1 second was determined by comparing two images taken 1 second apart with the variable minimum shutter speed (open triangles Δ).

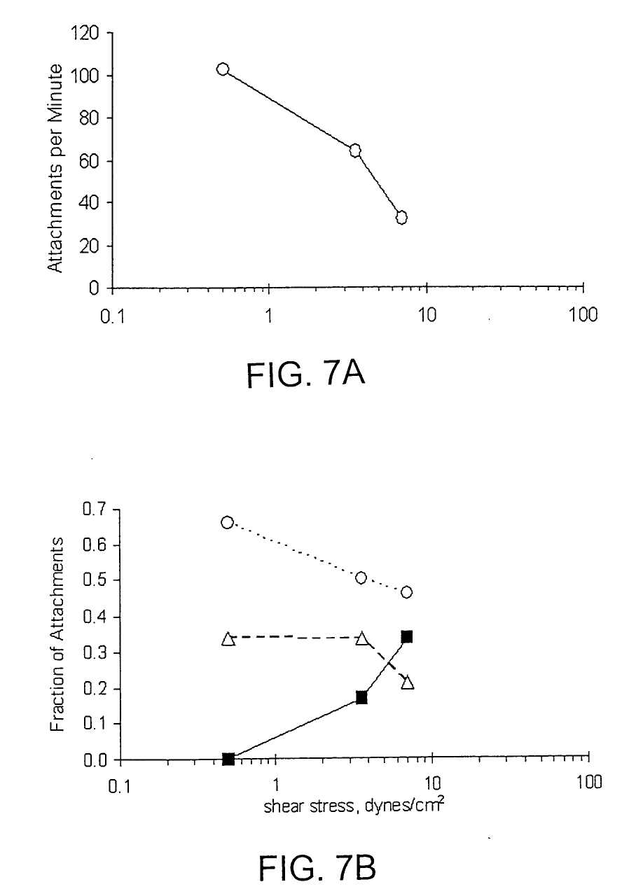

### FIGS. 7A-B.

Attachment rate of E. coli to 1Man-BSA surfaces. (FIG. 7A) Attachment was measured by counting the rate at which new bacteria appear in images taken every half-second for five minutes with a variable shutter speed. (FIG. 7B) Each E. coli was tracked as it rolled or remained stationary for at least 30 seconds or until it detached. When a bacterium rolled out of the field of view, a bacterium rolling into the field of view was chosen at random to replace it. Bacteria that bound for less than one second were classified as transiently binding (open circles ∘), from 1 to 30 seconds as short-term binding (open triangles A), and over 30 seconds as long-term (closed squares ▪).

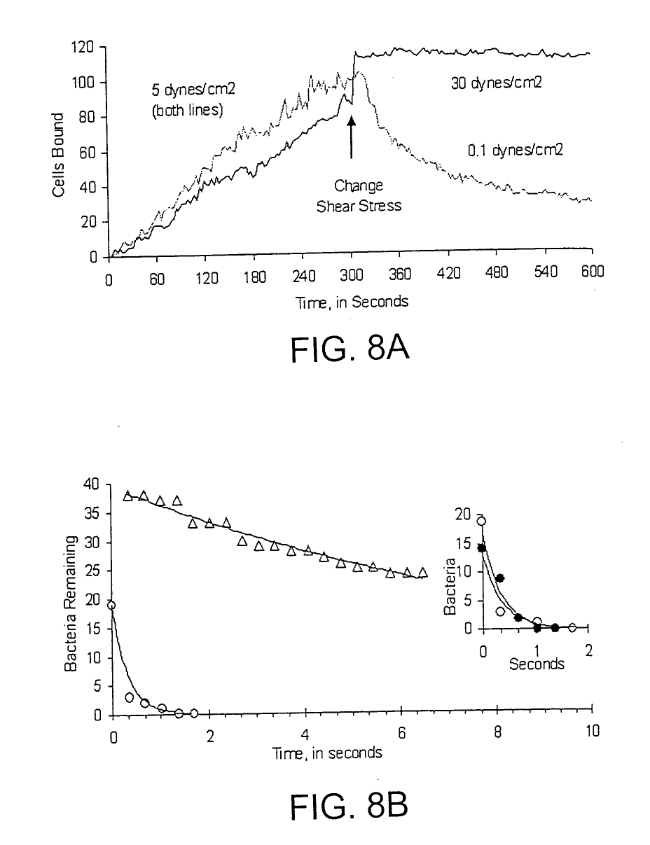

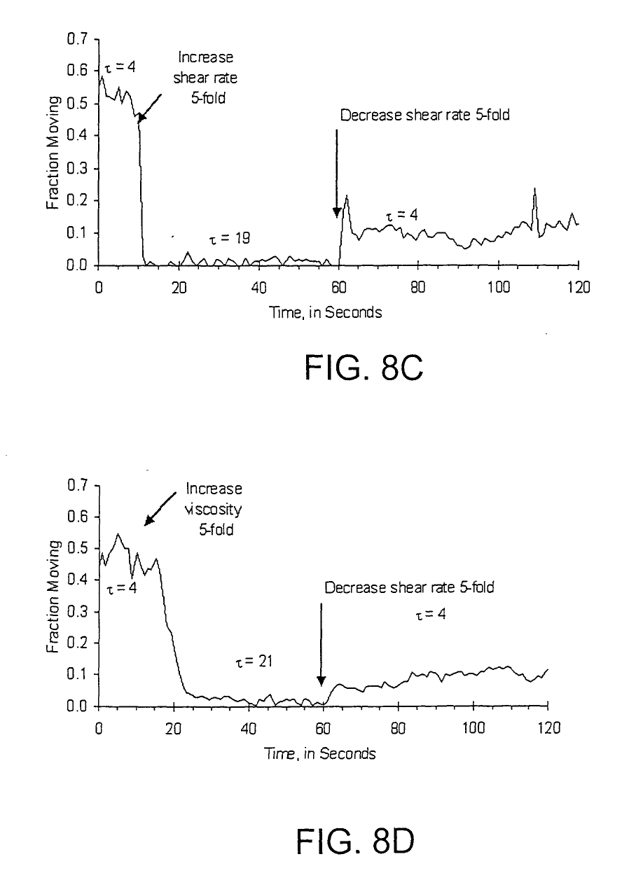

### FIGS. 8A-D.

Effect of changes in shear stress on E. coli bound to 1Man surfaces. (FIG. 8A) Bacteria were accumulated at 5 dynes/cm² for 5 minutes before being switched to either 0.1 (grey line) or 30 (black line) dynes/cm². Both videos were taken using a 750 ms shutter in order keep the free-floating bacteria at the lowest shear from obscuring visibility, but this prevented observation of most rolling bacteria at 5 dynes/cm². (FIG. 8B) A repeat of the switch from high (5 dynes/cm²) to low (0.12 dynes/cm²), was performed so that all bacteria were observed. Bacteria were attached to 1Man at high shear stress (5 dynes/cm²), washed for 30 seconds at the same flow rate to remove the unbound bacteria that would otherwise obscure the view, then brought to 0.12 dynes/cm² while taking a video with a fast (20 ms) shutter speed at ⅓ second intervals. Each cell was tracked and classified as stationary, rolling, or detached according to the distance moved in each frame. The cells that were rolling at the moment of change detached immediately (open circles ∘) while the cells that were stationary at the moment of change switched to a rolling state much more slowly (open triangles Δ). These newly rolling cells also immediately detached, at the same rate as did those that were rolling at the moment the shear stress was decreased (inset, closed circles ● vs. open circles ∘). The lines show first-order rate constants of 0.09 sec⁻¹ for the switch from stationary to rolling and 3 sec⁻¹ for the switch from rolling to detached. The rate of loss of stationary cells here is comparable to that in panel A. (FIG. 8C) Effect of increase in shear stress on rolling cells. Bacteria were accumulated on 1Man-BSA surfaces at 4.3 dynes/cm² for several minutes. Then, 10 seconds after starting video acquisition, the pumps were switched to a bacteria-free buffer with a 5-fold higher flow rate (19 dynes/cm²) an after 60 seconds, decreased 5-fold again to achieve the original shear stress. In the figure, tau indicates the shear stress in dynes/cm² during each time period. The number of bacteria moving at least one half cell diameter was measured each second by subtracting sequential images, and this compared to the total number of cells in each image to calculate the percent of moving bacteria. (FIG. 8D) Effect of viscosity. This experiment was performed the same as panel C, but a 5-fold more viscous buffer with 10% polyethylene glycol was used instead of changing the flow rate to get 21 dynes/cm². The results are essentially the same except that there was a delay between the pump change and the drop in bacterial mobility that reflects the time for the new viscous solution to move from the junction in the tubing to the imaged area.

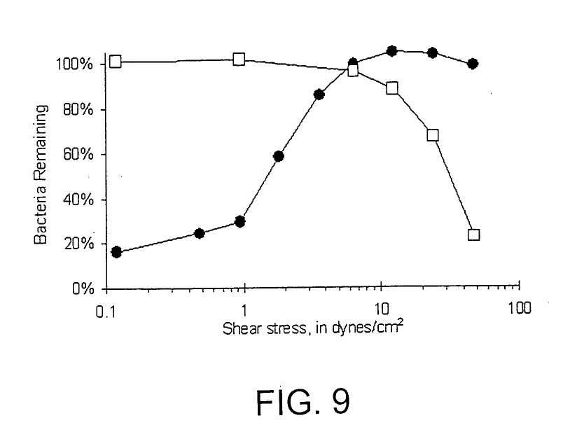

### FIG. 9.

Effect of shear on bacterial detachment from 1Man-BSA and anti-FimH. Bacteria were loaded onto surfaces of 1Man-BSA at 4 dynes/cm² (closed circles ●) or of anti-FimH antibodies at 0.1 dynes/cm² (open squares □) until about 200 to 400 bacteria were bound in the field of view, where upon the free-flowing bacteria were washed out with fresh solution at the same flow rate. The flow rate was then changed to the shear stress indicated in the figure, and the bound bacteria imaged with a variable short shutter time as in FIG. 6B in a time-lapse video. Bacteria were counted just before the change in shear stress and one minute after the change in shear stress in order to calculate the percent of bacteria remaining after one minute. Curve will dip down again at high shear stress.

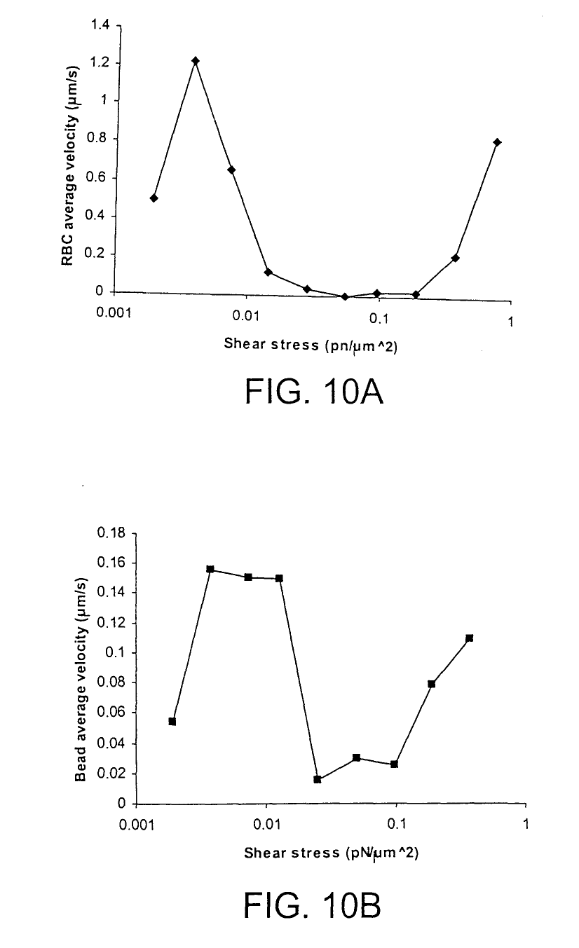

### FIGS. 10A-B

Effect of shear on the binding properties of red blood cells (RBC) over a carpet of either (FIG. 10A) E. coli or (FIG. 10B) E. coli fimbriae. Both the average cell velocity and the cell detachment rate is reduced at medium shears (0.01 to 0.1 pN/μm²) indicating that the bonds are shear activated in both cases. Note that 1 N/m² =1 pN/μm²=10 dynes/cm².

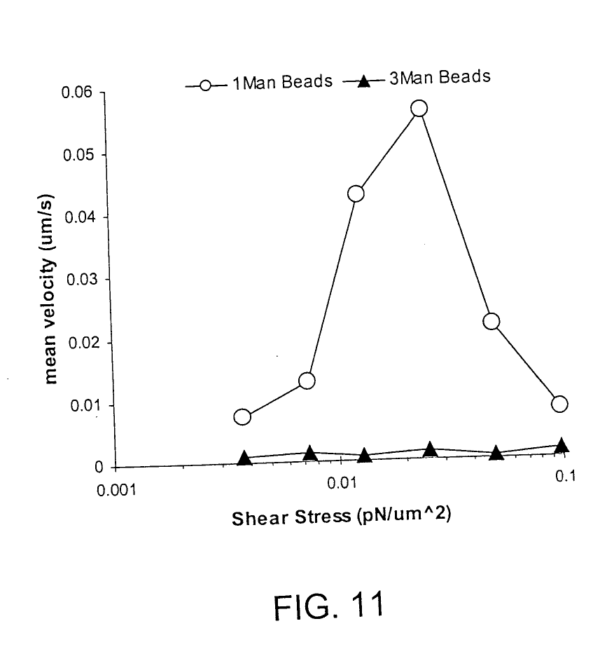

### FIG. 11

Comparison of the movement of 6 μm PS beads coated with 1Man (open circle ∘) and 3Man (solid triangle ▴), respectively, over a carpet of f-18 fimbriae in a parallel plate flow chamber. The bead velocity starts to drop at 0.03 pN/m². This assay proves that 1Man and fimbriae are sufficient to induce shear-activated adhesion. As a control, 3Man was used which adheres firmly to FimH in the full range of conditions chosen here.

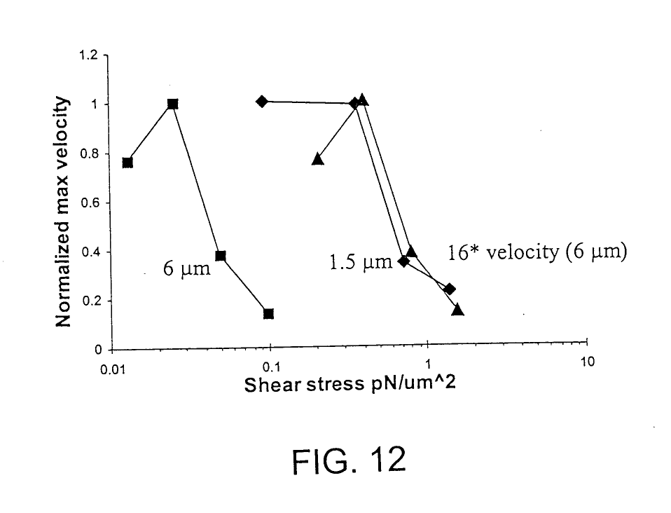

### FIG. 12

Relative particle velocity for 1.5 μm (solid diamond) and 6 μm beads (solid square ▪) coated with 1Man over a fimbrial carpet. Both sets of beads show shear-activated adhesion, but shear-activation occurs at different shear stresses. This indicates that it is not the shear stress that causes bond activation, but rather the drag force imparted on the particles by shear stress. This is confirmed by multiplying the velocity curve of the 6-μm beads by a factor of 16, i.e., the square of the ratio of the radii (solid triangle ▴), where we see a nice overlap with the velocity curve of the 1.5-μm beads. Relative particle velocity is the average particle velocity over the maximum average particle velocity of that experiment.

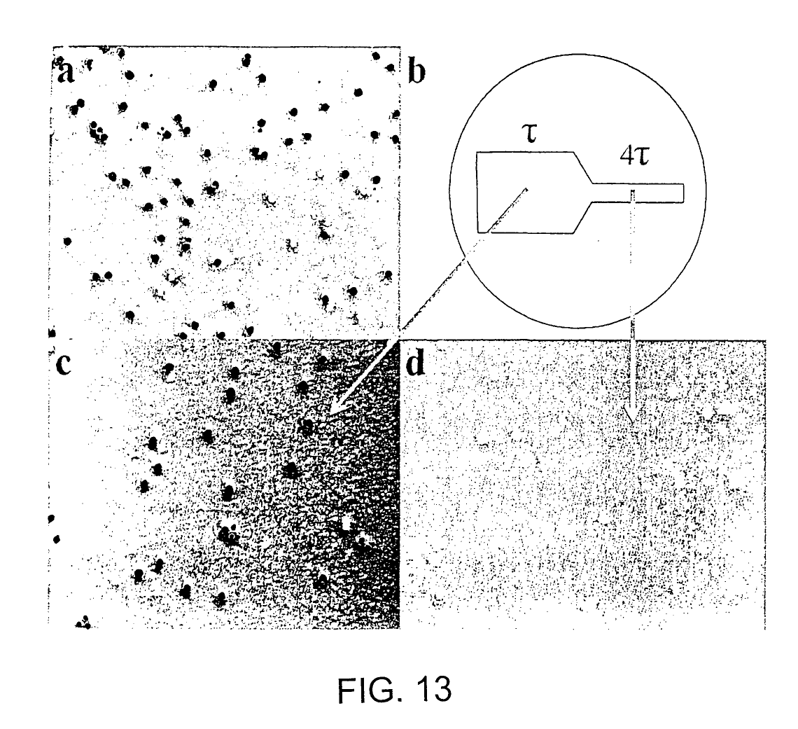

### FIGS. 13A-D

(FIG. 13A) Solution with initially 3 μm beads (white) and 6 μm beads (solid circle) are seeded on the surface of a fluidic chamber (FIG. 13B) that has a region of low shear (τ) and high shear (4τ) as indicated above. The chamber is of the same type as in all other experiments and the low shear region is 10 mm wide while the high shear region is 2.5 mm wide. Buffer solution flows from the large to the narrow section. The images are taken after the surfaces have been exposed for 5 minutes to a shear stress of τ=0.1 pN/μm² (FIG. 13C), and 0.4 pN/μm² (FIG. 13D). From the initial ratio of 45% small and 55% large beads, at a particle ratio of 45/55, the low shear region is depleted of small beads (only 15% left) because there is not enough force to activate their bonds. The high shear region is depleted of large beads (only 1% left) because the large shear creates sufficient force to washes them off.

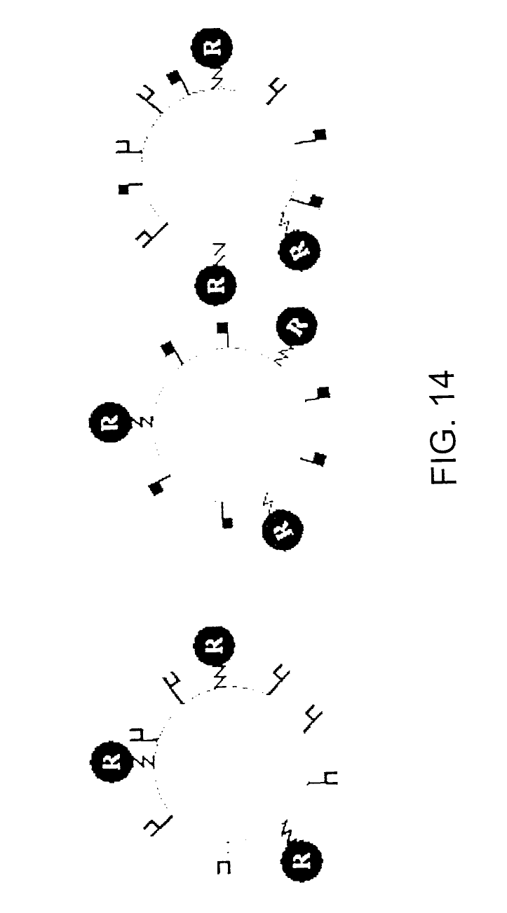

### FIG. 14:

Three alternative designs show how system A and/or B, respectively, can be functionalized with adhesins (open μ) and/or their respective ligands (closed square), potentially in combination with exposing other surface chemistries (R). While spheres are shown in the figure, our invention is not limited to spherical objects and includes any biological or nonbiological object of any size, shape or geometry, from infinitely flat, to complex shapes whose surfaces are functionalized with adhesins and/or their respective ligands. The spheres can represent a variety of objects including molecules, particles, cells, or clusters thereof . “Functionalization” with respective ligands and/or receptors can be accomplished by many approaches. This includes but is not limited to exposing ligands and/or their receptors on (a) cell surfaces, (b) synthetic surfaces after ligands and/or receptors are chemically cross-linked to reactive surface groups, and (c) biological and or synthetic surfaces after ligands and/or receptors are physisorbed (stabilization by formation of non-covalent bonds).

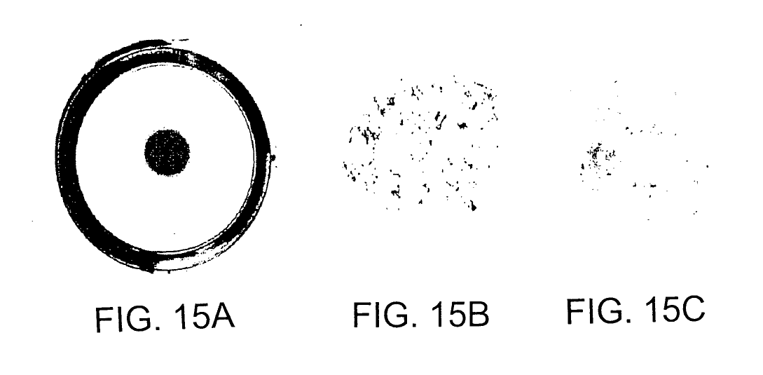

### FIGS. 15A-C.

Agglutination of RBCs by E. coli in static and dynamic conditions. (FIG. 15A) Bacteria expressing FimH-f18 do not form rosettes with RBC, but instead pellet to the bottom of round bottom wells. (FIG. 15B) When an identical mixture of FimH-f18-expressing bacteria and RBCs as in (A) are subjecting to rocking, they from tight aggregates. (FIG. 15C) After 3 minutes, the aggregates in (FIG. 15B) have loosened.

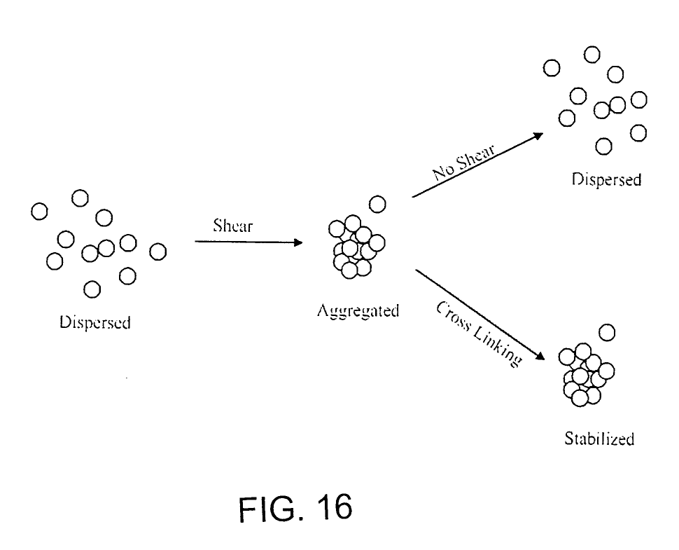

### FIG. 16.

Particles functionalized with adhesins and/or their respective ligands, as well as chemical groups that bind selectively to ions or molecules, including pollutants, drugs, vaccines, etc. (as shown in FIG. 14) are dispersed in solution under no shear, and aggregated under shear. Once shear is reduced, the aggregates disperse as the adhesin switches from high to low affinity. Separation processes thus have to be done either under shear, or within the critical time window prior to dispersion, or after the aggregates have been stabilized by other means.

### FIGS. 17A-D.

Some representative tracks of RBCs bound to FimH-f18-expressing E. coli are shown here under a shear stress of (FIG. 17A) 0.037 dynes/cm², (B FIG. 17) 0.55 dynes/cm², and (FIG. 17C) 7.20 dynes/cm² (1 dyne/cm²=0.1 N/μm²=0.1 pN/μm²). Each track shows 3 min total time with images taken at 10 s time intervals. The arrows show the path of a single cell while surface attached, while the arrowheads point to cells that did not move during the 3 min video at that shear stress. Movement of RBCs bound to a carpet of E. coli under shear in a parallel plate flow chamber. In our example, we used a Glycotech® parallel flow chamber just to illustrate this general effect. Some representative tracks of RBCs bound to FimH-f18-expressing E. coli are shown here under a shear stress of (FIG. 17A) 0.037 dynes/cm², (FIG. 17B) 0.55 dynes/cm², and (FIG. 17C) 7.20 dynes/cm (1 dyne/cm²=0.1N/m²=0.1 pN/mm²). Each track shows three minutes total time with images taken at 10-second time intervals. The yellow arrows show the path of a single cell while surface attached, while the yellow arrowheads point to cells that did not move during the three-minute video at that shear stress. The movement at each shear was then analyzed (FIG. 17D).

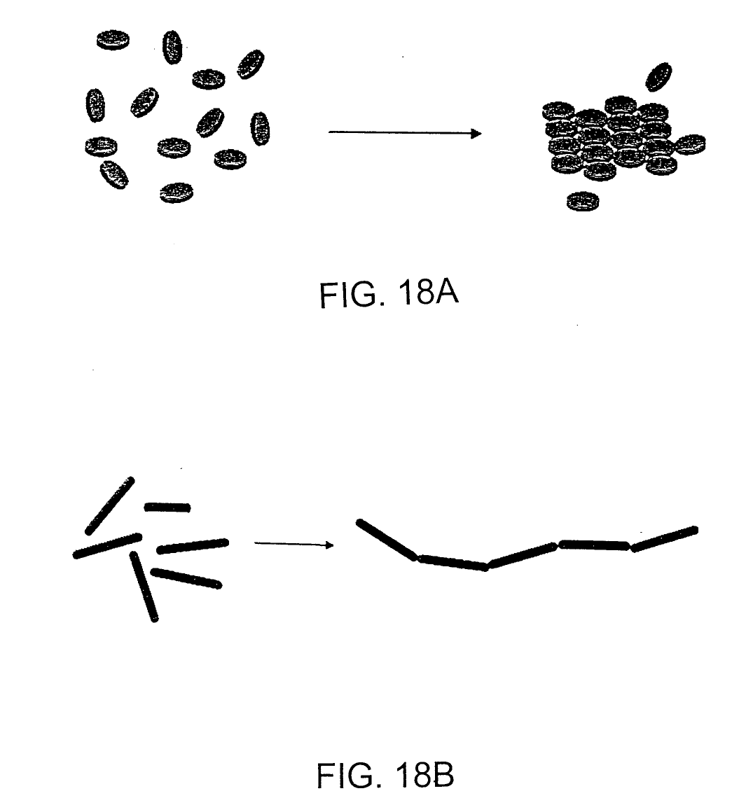

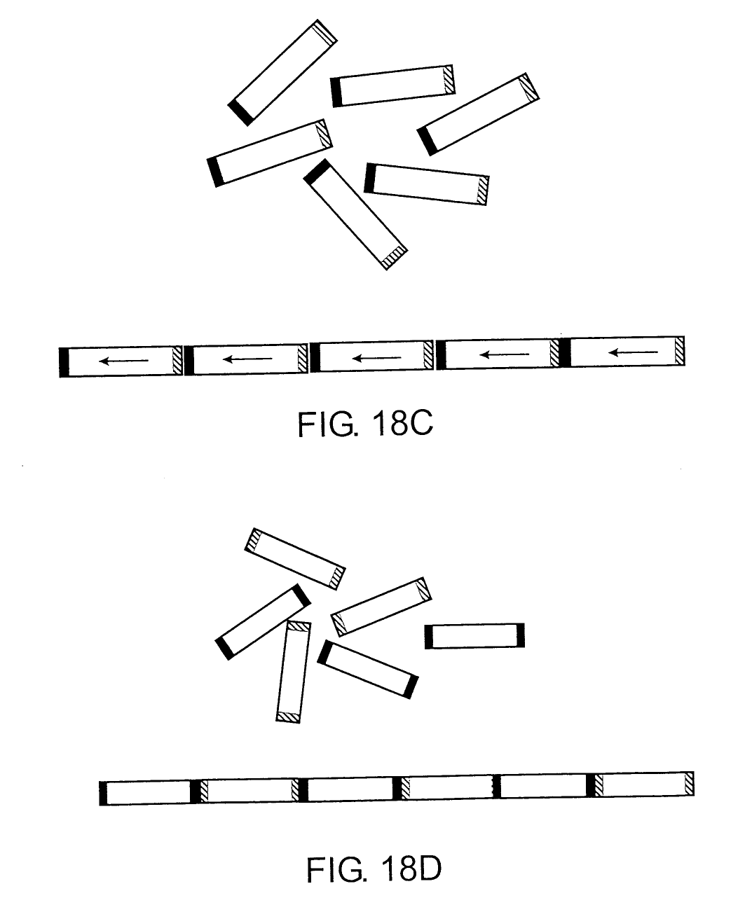

### FIGS. 18A-D:

(FIG. 18A Flat cylinders in solution whose edges are coated with FimH or other adhesins are exposed to shear and form a two dimensional membrane. (FIG. 18B) Long rods in solution whose caps are coated with FimH or other adhesins are exposed to shear and form chains. Note: particles in the above text refer to macroscopic, microscopic and nanoscopic particles or large molecules. (FIG. 18C) Cylinders in solution, wherein one end of each cylinder is coated with FABSDAMs and the other end is coated with FABSDB-L, are exposed to bond stress to form directional chains. (FIG. 18D) Cylinders in solution, wherein one subset of the cylinders have flat ends coated with FABSDAMs and another subset of the cylinders have flat ends coated with FABSDB-Ls, are exposed to bond stress to form alternating link chains.

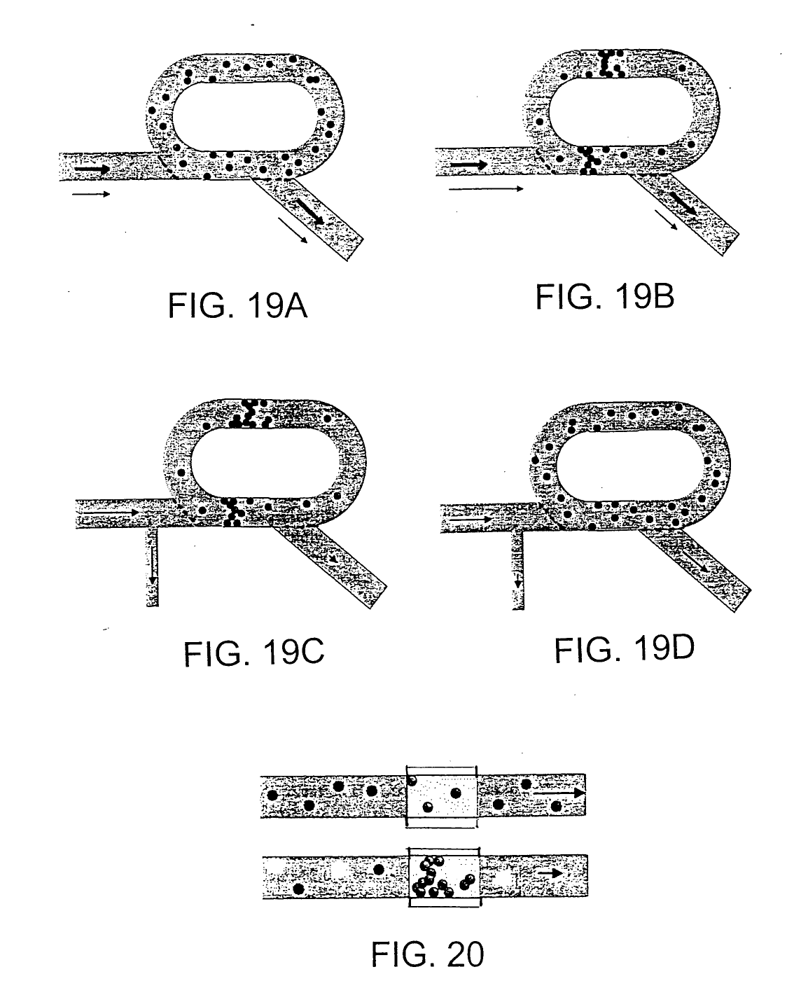

### FIGS. 19A-D.

A pressure-regulated microvalve that takes advantage of shear-activation. At low pressure (FIG. 19A, pressure indicated by heavy arrows), the fluid flows through slowly (indicated by narrow arrows), and the particles do not agglutinate, so the valve is open. At high pressure (FIG. 19B), the fluid begins to flow more rapidly, causing agglutination of the particles, which reduces the flow. Thus, aggregation regulates the fluid flow. One approach to recycle the particles to repeatedly and reversibly regulate the pressure is to keep the particles inside an optional recirculating channel by obstacles that pass the fluid but not the particles (dashed black lines). FIG. 19C-D: A shear-sensitive flow switch. With the addition of a narrow bypass route to the valve of FIGS. 19A-B, most of the fluid will go through the valve at low flow rates (FIG. 19C) but at higher pressures and flow rates, the valve with close, and most of the fluid will go through the bypass (FIG. 19D).

### FIGS. 20A-B

An externally controlled on-off valve. It is also possible to agglutinate the particles with external control (light boxes). Force can be created by a mechanical actuator transmitting vibrations in the channel, or by electric, dielectric or magnetic forces acting on the particles. The excitation of the particles will result in agglutination and/or in sticking to the walls and thus constriction of the channel and closing of the valve (FIG. 20B). In this particular setup beads are not recirculated but are inserted with the fluid. The flow in a flow channel can then be restricted on demand at any desirable position.

### FIGS. 21A-B

FIG. 21 A: FABSDB-L velocity is plotted as a function of bond stress. In this system, a plurality of FABSDAMs is in a fixed position on a substrate, and a plurality of FABSDB-Ls is in a fluid in contact with said FABSDAMs. As the flow of the fluid past the FABSDAM is increased, the FABSDB-Ls increase in velocity until the lower force-activated bond stress-dependent binding threshold (1) is reached. Point 4 on the graph is the lower threshold maximum. As the fluid flow increases, bond stress increases, and the velocity of FABSDB-Ls decrease as they bind to the FABSDAMs in a force-activated bond stress-dependent manner, until the bond stress reaches an upper force-activated bond stress-dependent threshold (2) is reached. Point 5 on the graph is the upper threshold minimum. At point 5 on the curve the FABSDB-L velocity can be zero. As the bond stress increases above the upper threshold, the FABSDB-L velocity reaches the same velocity as at point 4, at the higher force-activated bond stress-dependent threshold (3). If the FABSDB-L and the FABSDAM used to generate the date for this graph were not capable of bonding in a force-activated bond stress dependent manner, the curve would instead approximate the path shown in section 7. Section 8 demonstrates a hypothetical trajectory of the curve describing increasing bond stress for a FABSDB-L/FABSDAM pair. In the practice of this invention, applying any bond stress above the lower threshold is useful for generating force-activated bond stress-dependent binding of a FABSDB-L/FABSDAM pair, as all portions of the curve to the right of point 4 demonstrate decreased velocity of the FABSDB-L at a selected bond stress compared to section 7. Maximum binding strength occurs at the upper force-activated bond stress-dependent threshold.

FIG. 21B: FABSDB-L and FABSDAM binding strength is plotted as a function of bond stress. In this system, a plurality of FABSDAMs is in a fixed position on a substrate, and a plurality of FABSDB-Ls is in a fluid in contact with said FABSDAMs. As the bond stress on the FABSDAMs is increased, the binding strength is decreased until the lower force-activated bond stress-dependent binding threshold (1) is reached. Point 4 is the lower force-activated bond stress-dependent binding strength minimum. As the bond stress increases, the binding strength increases, eventually reaching an upper force-activated bond stress-dependent binding threshold (2). Point 5 is the upper force-activated bond stress-dependent binding strength maximum. As the bond stress increases, the binding strength decreases, eventually reaching a higher threshold (3) at point 6 wherein the binding strength is the same as at the lower threshold (1). If the FABSDB-L and the FABSDAM used to generate the date for this graph were not capable of bonding in a force-activated bond stress dependent manner, the curve would instead approximate the path shown in section 7. In the practice of this invention, applying a bond stress above the lower force-activated bond stress-dependent binding threshold (1) is useful for generating force-activated bond stress-dependent binding of a FABSDB-L/FABSDAM pair, as all portions of the curve to the right of point 4 demonstrate higher binding strength at a selected bond stress compared to section 7.

### EXAMPLES

### Methods for Examples 1-7

### Reagents

Monomannosylated BSA (Man1-BSA) was obtained from EY Laboratories, Inc. (San Mateo, Calif.). Guinea pig red blood cells (RBCs) were purchased from Colorado Serum Co. (Denver, Colo.). All other reagents were obtained from Sigma (St. Louis, Mo.).

### Bacterial Strains and Plasmids

Recombinant strains utilized here were constructed using a fim null K-12 derivative, AAEC191A (provided by Dr. Ian Blomfield, University of Kent, UK), and were described previously (Sokurenko et al., 1995). AAEC191A was transformed with the recombinant plasmid pPKL114 (provided by Dr. Per Klemm, Danish Technical University, Copenhagen, Denmark) to create strain KB18. Plasmid pPKL114 is a pBR322 derivative containing the entire fim gene cluster from the E. coli K-12 strain, PC3 1, but with a translational stop-linker inserted into the unique KpnI site of the FimH gene. Strain KB 18 cells express no fimbriae or very few numbers of long, nonadhesive fimbriae. For the studies reported here, strain KB18 was cotransformed with a series of isogenic pGB2-24-based plasmids. Plasmid pGB2-24 is a pACYC 184 derivative used for expression of various FimH alleles under a promoter. Recombinant strains created using these plasmids express large numbers of fully functional and morphologically identical type I fimbriae. Site-directed mutagenesis was performed essentially as described previously (Beck and Burtscher, 1994).

### Binding Assays

Assays of bacterial adhesion to Manl-BSA and bovine RNAseB immobilized in 96-well plates were carried out as described previously (Sokurenko et al., 1995). Briefly, Man1-BSA and bovine RNAseB were dissolved at 20 μg/ml in 0.02 M bicarbonate buffer, and 100 μl aliquots were incubated in microtiter wells for 1 hr at 37° C. The wells were then washed three times with PBS and quenched with 0.1% BSA in PBS. 3H-thymidine-labeled bacteria were added in 0.1% BSA in PBS and incubated for 40 min at 37° C. without shaking to achieve saturation, and the wells were then washed with PBS. The individual wells were subjected to scintillation counting. The density of bacteria used in all assays was 5×107 colony forming units per 100 μl. RBC rosette-formation assay was performed by mixing equal amounts of serially diluted bacterial suspensions (starting from OD540 nm=1.0) and a 1% suspension of RBC in U-bottom microtiter plate wells. On-slide agglutination assays were performed by mixing the suspension of RBC and bacteria on a slide surface followed by rocking at ˜3 s⁻¹.

### Parallel Plate Flow Chamber Experiments

Bacteria-coated dishes were prepared as follows: 35 mm tissue culture dishes were incubated with 20 μg/ml RNAse B in 0.02 M bicarbonate buffer for 1 hr at 37° C. and washed three times in PBS with 0.1% BSA (PBS-BSA). The dishes were then incubated with 200 μl PBS-BSA containing 108 colony-forming units of E. coli for 1 hr at 37° C. and washed three times. The E. coli bound through interaction of FimH with RNAse B (see Table 1) and FimH-negative bacteria did not bind significantly to the dishes. In all other variants, the E. coli were observed to form a confluent carpet of bacteria after this treatment, which was not perceptibly altered during the course of the flow chamber experiments. The dishes were then placed under a Glycotech parallel plate flow chamber with a size B gasket (2.5 cm×0.25 cm×250 μm) and sealed with vacuum. A Harvard model #975 pulse-free syringe pump was used to pump fluid through the chamber. The dishes were equilibrated in the flow chamber with PBS-BSA, and a 0.1% solution of RBC was injected into the chamber, allowed to settle onto the bacterial carpet, and washed with PBS-BSA at a shear stress of about 0.5 dynes/cm² until all free cells had been removed from the chamber and upstream tubing. The volumetric flow was then reduced to bring the shear stress down to 0.01-0.02 dynes/cm², and the shear stress was stepped up 2-fold, with at least 3 min at each shear stress.

RBC movement was recorded using phase contrast microscopy with a 20× objective, a CCD camera, and Metamorph video imaging software by Universal Imaging. Videos were recorded at 1 frame every 2 s for a total of 3 min at each shear rate. The position of each RBC was tracked at 6 s intervals in these videos using Metamorph's point tracking plug-in. The average velocity for each cell was calculated from these positions over the 3 min at each shear. These were then averaged for the average velocity of all cells. Some cells detached completely at the low shear stress and moved at the fluid velocity. (A cell moving in flow near a surface has velocity approximately equal to the cell radius, 5 μm, times the shear rate, assuming it is almost touching the surface.) Since these times reflected movement without any bond detachment, these time intervals were removed from the analysis for each cell. We only analyzed cells that were attached and in the field of view at the start of the 3 min time frame. The off-rate of RBCs was calculated from the X, the percent of original RBCs remaining bound at time t, using the formula k=1/t·1n(1/X), and using t=3 min. This assumes that detachment is independent and governed by a single rate constant, and X=e−kt, so that the number of remaining RBCs decays exponentially.

### Steered Molecular Dynamics

Steered molecular dynamics (SMD) simulations were performed using NAMD 2.3, which was developed by the Theoretical Biophysics Group in the Beckman Institute for Advanced Science and Technology at the University of Illinois at Urbana-Champaign (Kale et al., 1999). Molecular dynamics was performed as described earlier (Krammer et al., 2002), except that particle mesh ewald summations were used to calculate electrostatic contributions beyond the 13 Å cut-off. In brief, the lectin domain of FimH (residues F1 to T158) was hydrated in a 54×54×100 Å³ periodic box of water molecules and equilibrated for at least 500 ps. For equilibration and the ensuing simulations under force, the system was coupled to a 310 K bath and was coupled anisotropically to a Berendson pressure piston set at one bar with a relation time of 1 ps and a compressibility factor of 4.5×10⁻⁵ bar. During the equilibration, the box relaxed to 51.6×51.6×95.6 Å³ and remained the same size within 0.3% during simulations. When hydrogen atoms were added to the crystal structure, a disulfide bond was assumed between residues C3 and C44, and residue H45 was assumed to be protonated because it was surrounded by negatively charged residues D47 and D100 in the crystal structure (Choudhury et al., 1999). This left a net zero charge in the system as required for particle mesh ewald.

To simulate the shear-induced tension between the cell bound receptor and the anchor from the lectin domain to the pilin domain, the C terminus of the lectin domain was pulled at a constant force in one direction while the 13 residues of the putative receptor binding site were pulled with an equivalent sum force in the opposite direction. Forces were applied to the C-α carbon of each residue and the receptor binding residues were assumed to be the 13 residues that interacted with the C-HEGA mannose analog in the crystal structure (residues F1, 113, N46, D47, Y48, I52, D54, Q133, N135, Y137, N138, D140, and D141). Each run lasted about 1000 ps, and the total force ranged between 600 and 1000 pN, with at least two runs at each force, where each run at the same force used starting structures from different times during equilibration. Each run contained 26,892 atoms and required 4 days of simulation time on a Scyld linux Beowulf cluster with 12 nodes running at 1.3 GHz for the 1 to 2 ns required.

### Methods for Examples 8-14

### Reagents

Monomannosylated BSA was purchased from EY Laboratories, Inc. Anti-FimH antibodies were obtained by immunizing rabbits with 18 kDa N-terminal part of FimH (encompassing all lectin domain of FimH) that tends to be naturally co-purified with FimH-FimC complex on the mannose-sepharose (Langermann, 1997)

Parallel plate flow chamber experiments. Dishes coated with purified components were prepared by incubating 35 mm tissue culture dishes with 100 ml of either 200 mg/ml man-BSA, 200 mg/ml gal-BSA, or a 50,000-fold dilution of polyclonal anti-FimH antiserum in 0.02M bicarbonate buffer for 75 minutes at 37C, and then washing three times with phosphate-buffered serum with 0.1% BSA (PBS-BSA). The dishes were then inserted into a 2.5 cm×0.25 cm×250 mm parallel plate flow chamber (GlycoTech). Bacteria expressing f18-FimH were prepared as described previously (W. E. Thomas, E. Trintchina, M. Forero, V. Vogel, E. V. Sokurenko, Cell 109:913-23 (Jun. 28, 2002)), and brought to 108 cfu/ml in PBS-BSA. 2% alpha-monomannose was added to the PBS-BSA in the studies of surfaces coated with anti-FimH antibodies in order to inhibit any potential mannose-specific interaction between the bacteria and the carbohydrate modifications on the antibodies. This solution of bacteria was flowed through the chamber using a Harvard syringe pump at various shear rates. The bound bacteria were recorded using a Nikon inverted microscope with a 10× phase-contrast objective, a Roper Scientific high-resolution CCD camera, and MetaMorph video acquisition software. The field of view was 500 mm by 380 mm with a resolution of 0.8 mm per pixel. Even when 400 bacteria bound per field of view, they covered only 1% of the surface area, so that any interactions between bacteria were minimal.

### Video Analysis

Videos of bound bacteria were analyzed using MetaMorph imaging software. Total bound bacteria in an image appeared as dark spots in phase-contrast and were counted using the automated cell counting package in MetaMorph. The moving bacteria in an image were identified by subtracting an image from another taken one second earlier so that the bacteria that moved after the first image appear as dark spots that can be counted (FIG. 1, FIG. 3A, FIG. 4). This number was compared to the total number originally counted in the first image to give the fraction of bacteria that moved (FIG. 1B 3C 3D. The movement may represent detachment or rolling. In FIG. 3B where even detached bacteria were visible due to the short shutter speed and low flow rate, bacteria were classified as detached when they moved faster than the hydrodynamic velocity (the shear rate times the bacterial radius of I mm), as stationary if they moved less than one pixel (the size of the larger fluctuations in bacterial position over time that occurred with no net movement), and as rolling for anything in between.

### Materials and Methods for Examples 15-20

Reagents: Monomannosylated BSA (1Man) was obtained from EY laboratories, Inc. (San Mateo, Calif.). Polystyrene Microspheres were obtained from Polysciences, Inc (Warrington, Pa.). Guinea pig red blood cells (RBCs) were obtained from Colorado serum Co. (Denver, Colo.). All other reagents including RNAseB (3Man) were obtained from Sigma (St. Louis, Mo.).

Bacterial strains and fimbriae: Fimbriae are sheared off by a homogenizer, followed by differential centrifugation and MgCl₂ precipitation as described in Sokurenko, E. V. et al., (1994) and other publications. The particular strain used was FimH f-18.

Physisorption of fimbriae to PS plates: 35 mm Coming (#430165) tissue culture dishes were incubated with purified fimbriae diluted 500 fold in 0.02M bicarbonate buffer at 37° C. for 1 hour, and then washed thrice in PBS with 0.1% BSA (PBS-BSA) to prevent nonspecific adhesion by the beads or RBCs to the dish. Other plates (Falcon) did not perform satisfactorily in the sealing of the flow chamber.

Bead coating by 1Man-BSA: Polystyrene microspheres were prepared by rotating a solution of 50 μl of 2.6% beads mixed with 150 μl of 20-200 μg/ml receptor in 0.02M Bicarbonate buffer for one hour at room temperature. Then they were spun twice and resuspended in fresh PBS-BSA. They were finally diluted down to 0.1% and injected in the chamber.

Parallel plate flow chamber experiments: The coated dishes served as the bottom plate in parallel plate flow chamber from Glycotech # 31-0001 (Rockville, Md.) using a silicon rubber gasket 20 mm long, 2.5 mm wide and 0.010 in thick. The fluid (PBS-BSA) was pumped through the chamber a by a #975 pulse-free syringe pump from Harvard Apparatus, Inc. (Holliston, Mass.). The movement of the beads and RBCs was recorded using an inverted Nikon TE 2000 microscope with a long working distance 1Ox phase contrast objective by means of a Roper Scientific(Duluth, Ga.) Cascade CCD camera.

Data Analysis: Images of the flow chamber were recorded every 3 seconds for 3 minutes at each shear rate. The positions of cells and microspheres were tracked every frame using the point tracking plug-in from Metamorph video imaging software by Universal Imaging Corp. (Downingtown Pa.). The average velocity was calculated by averaging the velocities of all particles that did not detach during the lapse of the experiment. Detached cells or beads were defined as ones that flow at over ⅔ the expected free flowing velocity at some point during the lapse of the recording. The expected free flowing velocity was calculated by multiplying the shear rate by the radius of the particle, assuming that it is barely touching the surface. Off rates of RBCs were calculated from the fraction X remaining after t=3 min, assuming an exponential decay, using the formula k=1/t 1n(1/X). Off rates of beads were calculated in the same manner for experiments comparing them to RBCs.

### Materials and Methods for Examples 21-33

Reagents: Monomannosylated BSA (1Man) was obtained from EY laboratories, Inc. (San Mateo, Calif.). Polystyrene Microspheres were obtained from Polysciences, Inc (Warrington, Pa.). Guinea pig red blood cells (RBCs) were obtained from Colorado Serum Co. (Denver, Colo.). All other reagents including RNAseB (3Man) were obtained from Sigma (St. Louis, Mo.).

Bacterial strains and fimbriae: Fimbriae are sheared off by a homogenizer, followed by differential centrifugation and MgCl₂ precipitation as described in (Sokurenko, E. V. et al., (1994)) and other publications. The particular strain used was FimH f-18.

Physisorption of fimbriae to PS plates: 35 mm Coming (#430165) tissue culture dishes were incubated with purified fimbriae diluted 500 fold in 0.02M bicarbonate buffer at 37 oC for 1 hour, and then washed thrice in PBS with 0.1% BSA (PBS-BSA) to prevent nonspecific adhesion by the beads or RBCs to the dish. Other plates (Falcon) did not perform satisfactorily in the sealing of the flow chamber.

Bead coating by 1Man-BSA: Polystyrene microspheres were prepared by rotating a solution of 50 μl of 2.6% beads mixed with 150 μl of 20-200 jig/ml receptor in 0.02M Bicarbonate buffer for one hour at room temperature. Then they were spun twice and resuspended in fresh PBS-BSA. They were finally diluted down to 0.1% and injected in the chamber.

Parallel plate flow chamber experiments: The coated dishes served as the bottom plate in parallel plate flow chamber from Glycotech # 31-0001 (Rockville, Md.) using a silicon rubber gasket 20 mm long, 2.5 mm wide and 0.010 in thick. The fluid (PBS-BSA) was pumped through the chamber a by a #975 pulse-free syringe pump from Harvard Apparatus, Inc. (Holliston, Mass.). The movement of the beads and RBCs was recorded using an inverted Nikon TE 2000 microscope with a long working distance 1Ox phase contrast objective by means of a Roper Scientific(Duluth, Ga.) Cascade CCD camera.

Data Analysis: Images of the flow chamber were recorded every 3 seconds for 3 minutes at each shear rate. The positions of cells and microspheres were tracked every frame using the point tracking plug-in from Metamorph video imaging software by Universal Imaging Corp. (Downingtown Pa.). The average velocity was calculated by averaging the velocities of all particles that did not detach during the lapse of the experiment. Detached cells or beads were defined as ones that flow at over ⅔ the expected free flowing velocity at some point during the lapse of the recording. The expected free flowing velocity was calculated by multiplying the shear rate by the radius of the particle, assuming that it is barely touching the surface. Off rates of RBCs were calculated from the fraction X remaining after t=3 min, assuming an exponential decay, using the formula k=1/t 1n(1/X). Off rates of beads were calculated in the same manner for experiments comparing them to RBCs.

### Example 1

### Red Blood Cell (RBC) Agglutination in Static and Dynamic Conditions

Red blood cells (RBCs) of guinea pig are the most commonly used model target cells for studying the functional properties of type I fimbriae. We compared the RBC-agglutinating ability of two naturally occurring FimH variants—a low-Man1 binding variant, FimH-f18, from intestinal E. coli strain F18, and a variant with increased level of Man1 binding, FimH-j96, identical to the one expressed by uropathogenic E. coli strain J96 that has been crystallized (Choudhury et al., 1999). FimH-f18 represents a structural variant that is the most common one among intestinal E. coli, while FimH-j96 differs from the FimH-f18 by A27V, S70N, and N78S substitutions (Sokurenko et al., 1995, 1998; Choudhury et al., 1999). The A27V substitution, i.e., presence of valine instead of alanine in position 27, is responsible for increased Man1 binding capability of this type of FimH (Sokurenko et al., 1995, 1997, 1998).

Two commonly used RBC agglutination assays were performed that utilize different shear conditions. Static conditions were achieved with rosette-formation assays, in which RBCs were mixed with bacteria in U-bottomed microtiter plate wells and allowed to settle undisturbed for 30 min. If no agglutination occurs, RBCs fall into a pellet in the bottom of the well (FIG. 15A), while agglutination results in a rosette of RBCs crosslinked by bacteria. Dynamic conditions were achieved with on-slide agglutination assays, in which the suspension of RBC and bacteria were rocked on a slide surface at 3 s⁻¹, and agglutination was indicated by clumping of RBCs by bacteria. After three minutes without rocking, the aggregate loosened (FIG. 15C).

Type I fimbriated bacteria expressing the FimH-f18 variant were unable to mediate RBC agglutination in the static rosette-formation assay, even at the highest concentration of bacteria used (10⁹ bacteria/ml, see Table 1A and FIG. 15A). In contrast, this FimH variant was able to readily agglutinate RBCs in the dynamic rocking assay, where RBCs formed tight clumps in 42±3 s at the highest concentration of bacteria (10⁹ bacteria/ml) (FIG. 15B) and still formed aggregates when the bacteria were 10-fold diluted (Table 1A). Interestingly, however, the aggregates formed by the FimH-f18 bacteria began to dissipate within 3 min after rocking was stopped (FIG. 15C) but reformed promptly if rocking was restarted. Therefore, the FimH-f18 variant requires dynamic conditions to agglutinate RBCs.

TABLE 1
FimH Mediated Man1- and Trimannose-Binding and Red Blood Cell (RBC)
Agglutination under Static and Dynamic Conditions

| | | Receptor- | | RBC | rocking |
|---|---|---|---|---|---|
| | | binding Man | | agglutination | (dynamic) |
| | FimH variant | 1′ | Trimannoset | rosettes (static)' | § |
| A | FimH-f18 | 2.0-0.4 | 21.2-2.5 | >1:1 | 42-3 sec |
| | FimH-j96 | 6.1-0.9 | 20.0-1.9 | 1:8 | 35-2 sec |
| B | FimH-j96-V27A | 2.3-0.5 | 22.0-3.5 | >1:1 | 40-4 sec |
| C | FimH-f18-V156P | 4.2-0.5 | 19.7-3.0 | 1:8 | 37-3 sec |
| | FimH-j96-V156P | 15.5-1.4 | 18.4-2.3 | 1:32 | 33-2 sec |
| D | FimH-f18- | 0.4-0.2 | 18.0-1.3 | >1:1 | 42-2 sec |
| | Q32L:S124A | 0.5-0.2 | 20.8-2.5 | >1:1 | 40-2 sec |
| | FimH-j96- | | | | |
| | Q32L:S124A | | | | |

The binding capability of several variants of bacteria was defined as explained below. all binding was >90% inhibitable by 50 mM α-methyl-mannoside.
(A) functional difference between FimH-f18 and FimH-j96;
(B) effect of the V27A reversion substitution in FimH-j96 on the RBC agglutination capabilities;
(C) functional effects of the V156P mutation predicted to increase static binding capabilities of FimH; and
(D) functional effects of the combined Q32L and S124A mutations predicted to decrease static binding capabilities of FimH.
′Man 1 binding was measured by the number of bacteria binding to a mannosylated BSA-coated microplate under static conditions, and is expressed at 106 colony forming units (cfu) well.
f Trimannose binding was measured by the number of bacteria binding to a bovine RNAse B-coated microplate under static conditions, and is expressed in 106 cfu/well.
'Binding to RBC in static conditions was measured as the highest dilution of bacteria that formed rosettes (1:1 is 109 bacteria/ml).
§ Rate of the RBC agglutination under the dynamic, rocking conditions was measured at the highest concentration of bacteria (109 bacteria/ml).

### Agglutination under Static and Dynamic Conditions

In contrast, bacteria expressing FimH-j96 were able to agglutinate RBC in static conditions. This variant was able to form RBC rosettes up to an 8-fold dilution (Table 1A). Under dynamic conditions, these bacteria aggregated RBCs at a slightly higher rate relative to the FimH-f18 variant (35±2 s at the highest concentration). Furthermore, the RBC aggregates induced by FimH-j96 remained stable indefinitely long after rocking was halted.

Taken together, these results show that the ability of type I fimbriated bacteria to agglutinate RBCs depends on the shear conditions applied and that this phenomenon is mediated by specific functional properties of the FimH adhesin manifested in the FimH-f18 variant. Importantly, expression in isogenic background of FimH variants with different Manl binding does not affect the percentage of fimbriated bacteria, fimbriae number per bacterial cell, fimbriae morphology, or amount of FimH incorporated into the fimbriae (Sokurenko et al., 1995, 1997). In all assays, agglutination (when it occurred) was inhibitable by 50 mM α-methyl-mannoside, indicating its FimH-specific nature.

### Example 2

### Shear Force-Dependent RBC Binding in Flow Chambers

To establish whether the differential pattern of RBC agglutination observed in example 1 was due to a distinct ability of shear to enhance adhesion of type I fimbriated bacteria to RBCs, we studied the adhesion of low- and high-Man1 binding variants to RBCs under well-defined shear conditions. Previous studies on the effect of shear on selectin or von Willebrand factor-mediated adhesion of lymphocytes and platelets have used flow chambers (Finger et al., 1996; Marchese et al., 1999). Using a similar approach, we immobilized bacteria expressing either FimH-f18 or FimH-j96 on the surface of a flow chamber coated with bovine 3Man-RNaseB, a model trimannose-containing substrate to which both FimH variants bind with equal affinity (see Table 1A). In brief, RBCs were allowed to bind to the bacterial carpet, and the unbound cells were removed by rinsing under moderate fluid flow shear (0.28 to 0.90 dynes/cm²). RBCs that remained attached to the bacterial carpet were then subjected to various shear conditions. As observed in the agglutination assays, the original attachment of RBCs to bacteria could be inhibited by 50 mM α-methyl-mannoside, indicating that the bacteria-RBC interactions were FimH mediated.

Under low shear conditions (from 0.01 to 0.14 dynes/cm² shear stress), the RBCs attached to the FimH-f18-expressing bacteria were found to bind weakly, such that the cells moved sporadically along the adhesive surface (FIG. 12). However, at moderate shear (0.28 to 0.90 dynes/cm²), these RBCs exhibited decreased mobility (FIG. 1D) eventually becoming firmly adhered to the FimH-f18 bacterial carpet (FIG. 12). Not only did fewer cells move at moderate shear, but those that did moved more slowly. When the shear was switched back and forth repeatedly between low and moderate shear levels, the cells started and stopped moving repeatedly, indicating that the process of adhesion enhancement under shear was reversible. Thus, the FimH-f18-mediated adhesion of bacteria to RBC is stronger under moderate shear than under low shear, i.e., is shear-dependent. Furthermore, because the RBCs adhered firmly to bacteria expressing FimH-j96 even under low shear conditions (FIGS. 1 and 12), the shear dependence demonstrated by the FimH-f18 variant is an adhesin-mediated phenomenon. At sufficiently high shear (>2 dynes/cm²), RBCs began to move on the bacteria expressing either FimH variant (FIGS. 19 and 1D) and at shears much higher than 10 dynes/cm², all RBCs detached from the bacterial carpet. Thus, the flow chamber results corresponded well to the RBC agglutination patterns, with both series of experiments indicating that FimH-f18 mediates stronger binding of bacteria to RBCs under high shear than under low shear conditions, while bacteria expressing FimH-j96 can bind RBCs strongly under both conditions.

### Example 3

### Shear-Induced Decrease in Off-Rate

Though shear force normally decreases bond lifetimes (Bell, 1978; Evans, 1999), in principle there are at least two explanations for how shear could increase bacterial adhesion in some instances. The increased relative fluid velocities could increase the rate of FimH-receptor bond formation (i.e., have kinetics effect) as demonstrated for L-selectin-mediated rolling of leukocytes on adhesive surfaces (Alon et al., 1997; Chen and Springer, 2001). An alternative mechanism would be that the shear-induced mechanical drag force on the surface bound cell could cause a high-affinity conformation of the receptor bound adhesin and thus decrease the bond off-rate. We thus asked whether either the bond on-rates are increased by shear or alternatively the bond off-rates are decreased by shear. We have studied the off-rates for the entire RBCs from the surface. While RBCs detached from FimH-f18 E. coli substantially at low shear (off-rate=on average 0.1 min-1 at 0.2 to 0.4 dynes/cm²), we find for our system that an increasing shear dramatically reduced the off-rate (0.03 min-1 at 0.7 dynes/cm2 and 0.002 min-1 at 0.14 dynes/cm²) until it was too low to measure in our assays at shear stresses above 0.2 dynes/cm2 because RBCs did not detach in these conditions (FIG. 1E). As with the cell mobility measurements, RBC detachment rates changed with shear in a reversible manner.

### Example 4

### Effect of Viscosity

In addition, to distinguish whether shear rate (and the increase in transport kinetics, in units time⁻¹) or, alternatively, the shear stress (and the force on cells, in units force/area) is critical for the shear-enhanced adhesion, we adjusted the viscosity of the medium in the flow chamber experiments. A solution of 6% Ficoll was used to increase the viscosity from 1.0 to 2.6 centipoise. Since shear stress is shear rate times viscosity, this should increase the shear stress and the drag forces on cells 2.6-fold without affecting the shear rate and fluid velocity. In the presence of Ficoll, RBCs were observed to bind more strongly at all shears. Moreover, they slowed down at the same shear stress with or without Ficoll, but not at the same shear rate (FIG. 1F versus FIG. 1G). This demonstrates that shear force and off-rates, not kinetic effects and on-rates, dominates the shear activation of the FimH-mediated adhesive interactions between RBC and the type I fimbriated bacteria.

### Example 5

### Prediction of Force-Induced Conformational Changes in FimH Structure

We hypothesized that the molecular mechanism of shear-dependent bacterial adhesion was based, at least in part, on the ability of the tertiary structure of mannose-bound FimH to respond to the applied shear force. However, the crystal structure of FimH-j96 cannot offer immediate insights as to how applied force could affect the tertiary structure of FimH. As mentioned above, the increased Man1 binding capability of FimH-j96 compared to FimH-f18 is due to the presence of a valine in position 27 (Sokurenko et al., 1995, 1997). The presence of valine in residue 27 also allows FimH-j96 to agglutinate RBC in static conditions, since a recombinant FimH-j96-V27A with a reversion to alanine in residue 27 shows similar shear dependence of RBC agglutination as the FimH-f18 variant (Table 1B). However, from the crystal structure of FimH-j96 it is unclear how the A27V could affect FimH function, because this residue is located far away from the putative receptor binding site (see FIG. 2A). Traditional high-resolution methods in biochemistry and biophysics, such as X-ray crystallography and NMR, can only determine equilibrium structures and structural fluctuations around equilibrium. Other methods such as atomic force microscopy, optical tweezers, and biomembrane force probes measure forces and end-to-end distances of proteins (Wang et al., 2001) or receptor bonds (Merkel et al., 1999), but cannot probe the structures at high resolution. However, a recent computational method has proven to be useful for predicting stretch-induced conformational changes with angstrom precision-steered molecular dynamics (SMD) where a known protein structure surrounded by explicit water molecules is stretched under an external force (Isralewitz et al., 2001; Vogel et al., 2001). Here, we use SMD simulations to investigate how an external force may change the FimH tertiary structure.

In order to predict force-induced conformational changes in FimH structure, SMD simulations were performed on the crystal structure of the FimH-j96 variant, which is the only FimH structure available at this time. Because computationally intensive simulations limit experiments to short time windows, high-level forces were used to force changes to occur at a correspondingly faster rate than in nature. FIG. 2A shows that the receptor binding residues (green) are in proximity to the N terminus of the lectin domain. On the opposite side of this domain, the C terminus (residue 158) connects the lectin domain to the pilin domain and thus the rest of the fimbria and bacterium. To simulate the shear-induced tension across the lectin domain, the lectin domain was hydrated in a periodic box of water molecules, equilibrated, and subjected to force. The C terminus was pulled at a constant force in one direction while the 13 residues of the putative receptor binding site were pulled with an equivalent sum force in the opposite direction, as indicated by the gold arrows in FIG. 2A. This was intended to simulate tension across the domain between the cell bound mannosyl receptor and the linkage to the pilin domain. The receptor itself was not included in the simulations because the existing crystal structure used a noncyclic substitute compound instead of a natural mannopyranose-based receptor (Choudhury et al., 1999). Similarly, we could not include the pilin domain in the simulations as it was cocrystalized with the chaperone protein (Choudhury et al., 1999), and its native conformation within the fimbial tip is unknown.

The largest change that occurred in the SMD simulations affected the amino acid chain connecting the lectin and pilin domains of FimH (FIG. 2B). In the native structure, the interdomain linker chain consisted of 157-159 PTG amino acids (Choudhury et al., 1999), while the 154-156 VVV residues of the lectin domain are stabilized by hydrogen bonds with residues 117-120 GVAI of the 9-10 loop and residues 26-28 PVV in the 3-4 loop (FIGS. 3A and 3B). In the simulations, the external force caused these hydrogen bonds to break (FIG. 3C) and residues 154 to 156 to pull away from the rest of the lectin domain, doubling the length of the linker chain that connects the lectin and pilin domains (FIG. 3A versus FIG. 3C). This change was observed in multiple simulations carried out at a constant force of 700 pN or above. In contrast to the interface region, the rmsd changes that occurred in the main β sheets of the FimH lectin domain upon application of force (1.1 A, FIG. 3B) were comparable to the fluctuations that occurred during equilibration (0.9 A).

Remarkably, the A27V substitution in FimH-j96 is located in this region of linker-stabilizing bonds (see FIG. 3), which were predicted by the SMD simulations to play a critical role in the force-induced conformational changes in FimH, suggesting that the linker extension might be a functionally relevant event. However, it is important to note here that force-induced linker chain extension would lead to other structural events in the FimH molecule. Specifically, it would eliminate contacts between the FimH lectin domain and the FimH pilin domain or other fimbrial subunits. These additional changes may not be observable in SMD simulations if they involve other domains for which suitable crystal structures are not available. Because the linker chain is likely to be only one step in a cascade of force-induced conformational changes, the A27V substitution that affects shear-modulated properties of FimH may either alter the extension of the linker chain itself or, alternatively, may affect other steps in this cascade. Besides A27V, many other mutations of similar location can also affect the receptor binding properties of FimH under various shear conditions. The importance of linker chain extension by engineering mutations in FimH that are predicted to affect this event can be tested by the skilled worker without undue experimentation using functional assays.

### Example 6

### Experimental Tests of the SMD Predictions

If the linker chain extension is indeed critical to the shear-enhanced adhesion, then structural mutations that allow the linker chain to extend more easily should result in a FimH variant that requires lower shear to enhance bacterial adhesion. According to the SMD simulations described above, reduction of the force required to switch the linker conformation should be achieved by eliminating the stabilizing bonds between residues 154-156 VVV in the linker chain and the surrounding loop regions. Each of the stabilizing bonds is a backbone hydrogen bond and can be eliminated by replacing the hydrogen-donating residue with a proline. The latter has a closed ring structure that lacks the nitrogen-associated hydrogen atom in the backbone. To determine whether elimination of linker-chain stabilizing hydrogen bonds would affect the pattern of shear-dependent E. coli binding to RBCs, we engineered the point mutations V154P, V155P, and V156P into the FimH-j96 and FimH-f18 variants and tested their binding to RBCs. For all three mutations, the trimannose binding function was entirely conserved (Table 1C), suggesting that they did not cause major structural changes in FimH. For both FimH-j96 and FimH-f18 variants, the most dramatic functional change was observed with a V156P mutant (Table 1C). This is the outermost residue of the anchored stretch of the linker chain and the bond destroyed by this mutation is the outermost bond of the three force-bearing bonds. Both FimH-f18-V156P and FimH-j96-V156P mutants were able to agglutinate RBCs in static conditions significantly better than the corresponding wild-types into which the mutations were introduced (Table 1C). In flow chamber experiments, FimH-f18-V156P mediated a dramatically stronger adhesion under low shear than did the FimH-f18 parent (FIG. 4A). Therefore, experimental evidence supports our prediction that eliminating hydrogen bonds critical to the linker chain extension reduces the amount of shear needed to enhance adhesion of FimH to RBCs.

### Example 7

### Engineered Mutants

To further test the hypothesis that the force-induced linker chain extension leads to stronger binding under shear, we used SMD simulations to design a mutant with the linker chain stabilized against extension. The goal was to determine whether such a mutant would require more force to enhance FimH adhesion, i.e., would have the opposite effect from the putative extension-facilitating mutation V156P. We concluded that we could not build a stabilizing disulfide bond into the linker chain without altering the native structure, because no residue pairs were properly positioned for this (determined by the MODIPY program [Sowdhamini et al., 1989]). However, SMD simulations predicted that we could indirectly stabilize the linker chain. In some SMD simulations, an alternative force response was observed that correlated with delayed linker chain extension, so that the structure seen in FIG. 3D was observed instead of that in FIG. 3C. Two hydrogen bonds spanning turns in the 3-4 and the 9-10 linker-stabilizing loops ruptured (red and blue arrows, FIG. 3A versus FIG. 3D), and one or both loops distorted and extended along with the linker chain, instead of separating from it (FIG. 3D, red and blue loops). If these SMD observations were correct, FimH lacking these two hydrogen bonds would require more force to switch the linker chain conformation and consequently to enhance adhesion. We thus made two mutations, Q32L and S124A, in the structure of both FimH variants. As expected, the most dramatic functional effect of the mutations was evident in the background of Fim-H-j96 variant that binds RBCs strongly under static conditions. In support of our hypothesis, each mutation individually and, especially together (Table 1D), eliminated the ability of the bacteria to agglutinate RBCs under static conditions. In the flow chamber experiments, the double mutant mediated a dramatically reduced adhesion at low shear relative to the FimH-j96 variant, but provided comparable attachment to RBCs at medium and high shear, thus showing shear enhancement (FIG. 4B). This shows that these mutations increase the force needed to induce the linker chain extension and thereby increase the adhesive strength.

The three assays used in this work−1Man and 3Man binding to FimH, RBC agglutination assays, and the flow chambers experiments—have different dependencies on binding kinetics. Nevertheless, when introduced into either FimH-f18 or FimH-j96, the V156P mutation increased Man1 and low-shear binding, while the Q32L/S124A decreased Man1 and low-shear binding in all three assay types. Therefore, our hypothesis that the linker chain between the lectin and pilin domains extends and leads to activation of bacteria-cell adhesion has generated two separate predictions that were experimentally verified here using structural mutations and the three functional assays.

### Example 8

E. coli bacteria specifically adhere to mannose which is displayed on the surfaces of a variety of mammalian cells. Bacterial adhesion and accumulation is the first step in colonizing, and in many cases infecting target tissues. In order to determine the mechanism which allows bacteria to adhere to target tissues under shear, we studied the kinetics by which E. coli bacteria attach and detach from 1Man-coated surfaces, and how the attachment and detachment behavior depends on shear. Recombinant type I fimbriated E. coli bacteria were used that expressed a variant of the adhesin FimH. FimH mediates weak binding to monomannose (1Man) in the absence of shear, and switches to high binding strength under flow as demonstrated in our earlier work (Thomas, 2002). This FimH variant is found in commensal E. coli strains, including the strain F-18 used in our study. Mannosylated bovine serum albumin (1Man-BSA) was adsorbed to glass to prepare chemically controlled model surfaces that expose only 1Man in contrast to using red blood cells that present additional ligands and receptors that might interacting with E. coli. Plain bovine serum albumin (BSA) alone does not interact with adhesion proteins in a specific manner. A suspension of E. coli cells was passed through a flow chamber coated with 1Man and the binding of bacteria to the surface under various shear conditions was determined. Using a long (2 second) camera shutter time to blur out the free floating cells, we measured the accumulation of stationary bacteria bound to the surface after a period of five minutes at a range of physiologically relevant shear stresses, from 0.12 to 20 dynes/cm (FIG. 6, triangles). At low shear (0.1 to 0.5 dynes/cm²), E. coli failed to attach and accumulate. This is consistent with previous reports of poor binding of this strain to 1Man in the absence of shear. FIG. 6 shows that an increasing number of bacteria accumulated on 1Man surfaces at shear stresses above 1 dynes/cm², peaking at around 3-5 dynes/cm² with over 100-fold higher binding, and then decreased in numbers so that little accumulation was measurable at 40 dynes/cm².

For most noncovalent bonds, it is expected that the bond lifetime is shortened as tensile forces typically lower the energy barrier(s) between the bound and unbound states leading to a “slip” behavior (Evans, E. (1999) “Looking inside molecular bonds at biological interfaces with dynamic force spectroscopy,” Biophys. Chem. 82:83-97; E. Evans, Annu Rev. Biophys. Biomol. Struct. 30:105-28 (2001)). In contrast to shortening of the lifetime, we observe that shear enhances the adhesion of E. coli to 1Man surfaces (FIG. 6). If galactose (galactosylated BSA) rather than 1Man is presented on the model surface, essentially no bacteria bound at any shear since galactose is not specifically recognized by FimH. This control confirms that shear-enhanced adhesion is mediated by specific FimH binding to 1Man. Antibodies are thought to bind antigens via a slip-bond mechanism which we could indeed confirm by coating the model surface with anti-FimH. In contrast to 1Man surfaces, shear inhibited rather than enhanced accumulation of bacteria on the anti-FimH surfaces (FIG. 6, squares). Thus, bacterial accumulation on surfaces with anti-FimH antibodies represents the case where bacterial adhesion is mediated by slip-bonds. Since accumulation on 1Man ligands is shear-activated for an otherwise identical system, our data illustrate that E. coli binding to purified 1Man occurs via the formation of one or more ‘catch-bonds’ between FimH and 1Man. We proposed earlier a structural mechanism by which FimH can be switched from low to high affinity for mannose if stretched.

### Example 9

In agreement with the accumulation studies, E. coli detachment from model surfaces coated with either 1Man or anti-FimH show an antagonistic shear dependency. We attached bacteria to either 1Man-BSA or anti-FimH antibody coated surfaces at the optimum shear stress determined for accumulation on the respective surfaces. Free-floating bacteria were then washed out with fresh solution at the same flow rate before switching to the shear stress indicated in FIG. 9. The bacteria detached from the antibody-coated surfaces with increasing shear stress, as expected for slip-bond mediated adhesion, and from the 1Man-BSA-coated surfaces with decreasing shear stress as expected for catch-bonds (FIG. 9). This again demonstrates that E. coli binds to the antibodies via slip bonds that increasingly break with shear, in contrast to FimH mediated binding to 1Man that is enhanced by shear.

### Example 10

Live observations revealed that a fraction of the surface-bound bacteria exists that rolls along the surface with velocities far below the hydrodynamic velocity, whereas other bacteria firmly adhere. In order to distinguish rolling bacteria from those that moved at hydrodynamic velocity, we opened the shutter for just the length of time it took for free-floating bacteria to blur. At the shorter shutter opening times, the rolling bacteria could be seen while the free-floating ones blurred, while at longer shutter openings all but the stationary bacteria blurred (FIG. 6B, open vs. closed circles). At the peak of the accumulation curve on 1Man, many bacteria rolled steadily at about 30 mm per second, or at about one tenth the hydrodynamic velocity. They often rolled continuously when they first bound, but then became stationary after random lengths of time. These stationary cells periodically jolted forward and stopped again or started rolling steadily once more. In some cases, rolling or stationary bacteria detached completely from the surface. In contrast, bacteria that accumulated at much higher flows were entirely stationary except for occasional short jolts forward. Thus, when the fraction of bound bacteria that were stationary during any one second period was quantified by comparing two images, this fraction increased with shear (FIG. 6B, triangles). The reduced movement of bound bacteria at higher shear stress shows that an increased fraction of bonds between FimH and 1Man has switched to high affinity. Accumulation of E. coli on 1Man was enhanced by shear up to about 3 dynes/cm² and dropped again at higher shear. Working at two different shutter speeds allowed us to distinguish rolling from firmly binding bacteria. The two populations could be distinguished if the free-floating bacteria had moved while the shutter was held open. Accordingly, the set of optimal shutter openings had to be adjusted to the flow rates.

### Example 11

The numbers of accumulated bacteria reflect a balance between the rate of binding and the residence time once a bacterium is bound, both of which could be affected by shear in different ways. We therefore investigated the rate of initial bacterial binding at several shear rates, as well as the length of time each bacteria remained bound, whether in a rolling or stationary mode. We found that the number of initial attachments to 1Man-BSA in the field of view decreased from 102 attachments per minute at 0.5 dynes/cm² to 64 at 3.6 dynes/cm², and an additional 50%, to 32 at 7 dynes/cm² (FIG. 7A). Thus shear actually decreased the rate of binding of E. coli to the 1Man surface. This may be either because shear decreases the near-surface concentration by washing away bacteria that have settled due to gravity, or because flow inhibits the inherent attachment rate, or potentially due to both effects. However, shear increased the residency time dramatically once bacteria had bound to the surface. We defined binding events that lasted less than 1 second as transient, between 1 and 30 seconds as short-term, and longer than 30 seconds as long term, whether the bacteria rolled or bound firmly during this time. At low shear (0.5 dynes/cm²), all adhesions were transient or short-term, so the lack of accumulation at low shear was due to a failure of E. coli to remain bound to 1Man-BSA (FIG. 7B). In contrast, at high shear stress, (2-7 dynes/cm²), many bacteria remained bound for long times (FIG. 7B). As shear stress further increased above 7 dynes/cm², the proportion of long-term adhesive events did not increase enough to make up for the drop in initial attachments, so the number of long-term attachments, or accumulation, was inhibited by shear as seen in FIG. 6. Thus shear enhances accumulation not by increasing the transport of bacteria to the surface or the intrinsic attachment rate, but by decreasing the detachment rate of the bacteria by increasing the probability of a switch from transient to long-term adhesion. This switch is consistent with an increase in bond lifetimes under the influence of force, which is an essential characteristic of catch-, but not slip-bonds.

If FimH functions as an affinity switch activated by shear, it is of considerable interest to know whether the switching is reversible, and if so, to determine the characteristic time scales. After 300 seconds of accumulation at an intermediate shear of 5 dynes/cm², the shear stress was switched down to 0.1 dynes/cm², a shear stress where we do not expect the bacteria to firmly bind to 1Man. The bacteria dropped gradually in numbers after switching to the low flow. If the shear stress was switched from 5 dynes/cm² up to 30 dynes/cm², a shear rate at which E. coli had shown firm adhesion, the number of firmly bound bacteria first increased, a process discussed below, and then remained constant over the remaining time period (FIG. 8A). The finding that the number of bacteria remains constant under high shear demonstrates that only the bacteria that had adhered to the surface prior to switching the shear flow were able to remain surface bound while additional bacteria adhesion was inhibited under these high flow conditions.

### Example 12

In order to determine the kinetics by which stationary cells switch to a rolling mode, and by which rolling cells detach, the experiment was repeated but with a fast shutter speed and after rinsing the surfaces for 30 seconds at 5 dynes/cm² prior of switching the flow to remove the unbound bacteria that would otherwise obliterate visibility at the low flow rate with the fast shutter. When E. coli were switched from 5 dynes/cm to 0.1 dynes/cm², all of the rolling bacteria immediately began moving at the hydrodynamic velocity, indicating that they had detached (FIG. 8B, open circles). The number of rolling cells decayed exponentially from the rolling mode to free-floating. The stationary bacteria transitioned much more slowly from stationary to rolling (FIG. 8B, closed triangles). The number of stationary bacteria declined linearly with time, with a first-order rate constant of about 3 sec⁻¹. In our experiments, we observed that most, if not all cells transitioned from the stationary to the rolling mode prior to detachment. Once they had transitioned into the rolling mode, they detached with the same kinetics (FIG. 8B, closed circles and insert) as the cells that were in a rolling mode at the time when we switched from 5 dynes/cm² to 0.1 dynes/cm² (FIG. 8B, open circles and insert). Thus the stationary and rolling bacteria appear to be in two distinct states. The resident time of the bacteria in the rolling state in our experiments is independent of the history by which E. coli entered the rolling state. In contrast, stationary bacteria begin to roll at stochastic time intervals at any shear stress. Once they started rolling, they detached at low shear, but jolted briefly forward before becoming again stationary at high shear.

### Example 13

When E. coli that had accumulated on 1Man at 4 dynes/cm² were switched to higher shear (20 dynes/cm²), the rolling bacteria immediately became stationary (FIG. 8C). This explains the jump in numbers in FIG. 8A, since many rolling cells could not be seen with the long shutter time. This transition was reversible; when the bacteria were exposed again to 4 dynes/cm², many started rolling again, although it were often different bacteria that now rolled and the total percentage of rolling bacteria remained lower. Thus, rolling bacteria detach at low shear, become stationary at high shear, and usually continue to roll at about 4 to 5 dynes/cm².

### Example 14

To confirm that the transition from rolling to stationary adhesion was due to an increase in drag force rather than to an increase in fluid velocity, we increased the shear stress by using a more viscous buffer instead of by using a higher flow rate. Again, the rolling bacteria became stationary except that the transition was more gradual and slightly delayed, reflecting the flow of the more viscous fluid into the chamber and to the field of view (FIG. 8D). This is also consistent with the notion that the shear enhancement is due to catch-bonds. Assuming Stoke's law, the drag force on a bacterium attached to the surface is 1.7*6pr2t (A. J. Goldman, et al., 1967), where r is the bacterial radius, and t is the wall shear stress. Assuming that the radius of the fimbriated bacterium is about 1 mm, the drag force on each bacterium is about 3 pN at 1 dynes/cm², 16 pN at 5 dynes/cm², and 64 pN at 20 dynes/cm².

### Example 15

Bacterial derived fimbriae can be used in a cell-free assay to mediate shear activated adhesion between nonbiological systems. In order to prove that the fimbriae once sheared-off the bacteria surfaces are still able to mediate shear-activated adhesion, we coated the bottom of a parallel plate flow chamber either with E. coli or alternatively with fimbriae isolated from a recombinant bacterial strand expressing FimH-f18). We either allowed RBCs, for comparison to our previous studies (Thomas, W. E., et al. 2002), or polystyrene beads coated with 1Man or 3Man to bind to the E. coli or fimbriae coated plates. The detachment properties of the remaining cells were measured at varying levels of shear (FIG. 10). The binding pattern of RBCs is essentially the same when they attach to purified fimbriae and when they attach to bacteria: At low shears (from ˜0.001 to ˜0.01 N/m2) the RBCs bind weakly to the fimbriae carpet (off-rates of k_{off}=3×10⁻³ s⁻¹) and move at average speeds of up to 0.3 μm/s. At moderate shear (0.02 to 0.1 N/m²), the RBCs bind more tightly to the fimbriae: their off-rate drops an order of magnitude to under k_{off}=2 10⁻⁴ s⁻¹, and the average speed drops to under 0.05 μm/s. At higher shears the RBCs roll similarly on fimbriae as on a bacterial carpet. The similarity between the binding patterns of RBCs over bacteria and over fimbriae strongly suggests that the presence of fimbriae is sufficient for shear-enhanced adhesion. It also demonstrates that purified fimbriae must contain both the force sensor and the molecular recognition element that switches from low-to-high affinity under shear. Consequently, no other molecules are involved in mediating shear-activated adhesion of E. coli to target cells, but FimH and 1Man.

### Example 16

### Shear-Activated Nanolog

To illustrate the possibility of using the adhesin FimH and 1Man immobilized on synthetic surfaces as shear-activated nano-glue, we tested whether polystyrene beads coated with 1Man (see Methods) bind in a force-activated manner to a carpet of fimbriae. FIG. 11 confirms shear-activated binding of 1Man-coated 6 μm polystyrene (PS) beads to a carpet of fimbriae. The average off-rate of the 1Man beads decreased with increasing shear from 0.03 to 0.1 pN/μm², indicating shear activation. These data also confirm that other properties of RBCs do not contribute to shear activation, and that 1Man is responsible for shear-activation. This is important to know, since RBCs have complex membranes exposing a variety of receptors and ligands on their surfaces, and have a compliant membrane from which membrane tethers can be pulled (Evans, E. et al., 1996).

### Example 17

### Size Separation:

Biological shear-activated switching can be exploited for technological applications by size-dependent sorting of beads. This use has considerable technological implications, including for separation technologies and in MEMS. The equation for Stoke's law of viscous drag is F=6πμvr=6πμSr². The drag force F exerted on a bead attached to a wall is proportional to the square of the bead's radius and the shear stress imparted by the fluid (Mascari, L. & Ross, J. M., 2001) according to the equation

F∝6πμrₚ²∝r²τ

Here μ is the viscosity of the fluid, r is the bead's radius, and τ is the shear stress. Therefore we expect that the ratio of the force for two beads of different sizes is given by the square of the ratio of the radii. For bead diameters of 1.5 μm and 6 μm, respectively, it takes 16 times more shear stress on the small beads to generate the same force as experienced by the large bead, since the ratio of their radii is 4. FIG. 12 shows the velocity of 1.5 μm and 6 μm beads moving in a parallel flow chamber whose surfaces are covered with equal surface concentrations of fimbriae. The surface concentration of 1Man on the beads is equal. As expected, considerably higher shear stress is required to induce shear-activated adhesion of the 1.5 μm beads. Instead, if we consider that shear activation scales with the force exerted on the beads and multiply the velocity curve obtained for the 6 μm beads by 16, we see that the curves overlap nicely as predicted by the theory. This result also shows that there is not a significant difference between the number of tethers formed between these two types of beads and the underlying fimbrial carpet. In atomic force microscope (AFM) images we indeed see that the fimbriae are spread out far Oust a few per 10 μm²) and do not aggregate significantly.

### Example 18

### Particle Sorting

To further illustrate utility of shear-enhanced adhesion in separation experiments, we sorted particles in different regions of a fluid device according to particle size (FIG. 13). Our flow chamber had two regions differing in width by a factor of 4 (one is 2.5 mm and the other 10 mm wide), making the shear stress 4-fold smaller in the wider region. A mixture of 3 μm and 6 μm beads was seeded in both regions, and after five minutes of exposure, the large beads kept holding on to the surface in the low shear region while they rolled away in the high shear region. As expected, the small beads did the opposite, they rolled away in the high shear regions and firmly attached in the low shear regions.

### Example 19

### Shear Thickening Fluid

We have shown that it is possible to aggregate beads covered with both fimbriae and 1Man receptors in the presence of shear, while keeping them unaggregated otherwise. This property can be used to design a dilatant fluid, whose viscosity increases with shear. This type of fluid is also known as a shear thickening fluid. One application of such a fluid is the design of light body armor (see http://www.asc2002.com/oral_summaries/A/AO-01.PDF). Current shear thickening fluids used in the cited applications work by using a high density of beads which raise viscosity through their steric interactions. One advantage of the use of particles coated with FimH and 1Man, or other receptor-ligand pairs that show shear-enhanced adhesion, as shear thickening fluids is that their binding interaction can complement the steric interaction, increasing the change in viscosity. Also, the shear threshold can be tuned by using different sizes of beads as seen above, and combinations of different size beads can cover a whole range of shears. It is possible to tune the dilatant fluid's properties by using different FimH strains or engineered FimH polypeptides, which we have found to activate at similar levels of shear but have higher (or lower) binding strength at low shear.

### Example 20

### Shear-Controlled Site-Directed Assembly of Nano Beads

We also show shear-directed assembly of particles on controlled surface regions. One approach is to pattern the surface with either the receptor (stress-dependent adhesion molecule) or ligand. In a sequential step, we accumulate particles on these designated surface spots, by coating the particles with ligands and receptors simultaneously. The particles then bind to the surface and aggregate among themselves in a shear-dependent manner to form larger aggregates in designated areas. The particle size can range from the nanoscale to macroscopic. Alternatively, another receptor or ligand is patterned to the surface to immobilize nucleation sites for the particles. For example, biotin or streptavidin can be patterned on the surface to hold a particle that is functionalized with the complement to biotin or streptavidin, and simultaneously with receptors and/or ligands that show shear activation. Nucleation then happens only around these particles, under a shear regime that is dependent on the particle/aggregate size.

### Example 21

### Use of FimH or Other Adhesins as a Non-Invasive Probe for Force Fields

For many industrial applications, as well as in biotechnology and biomedical devices, it is of interest to have markers available by which flow fields can be probed non-invasively. Engineered probe particles will be injected or added to the fluid that flows through the system or device of interest. The probe particles will be functionalized with adhesins and ligands, respectively, such that they agglutinate (aggregate) reversibly under well-defined flow conditions. Depending on the size-scale of the device, in which the flow conditions are to be probed, particles will be sized appropriately. Many alternate approaches can be used to probe the particle aggregation or agglutination non-invasively, including by imaging the presence of aggregates, or by the use of light scattering or other optical or electrical techniques.

To illustrate this principle, E. coli serves as system A, and RBC as system B. The principle of shear-induced aggregation between two types of particles is illustrated by performing two commonly used RBC agglutination assays that utilize different shear conditions. Static conditions were achieved with rosette-formation assays, in which RBCs were mixed with bacteria in U-bottomed microtiter plate wells and allowed to settle undisturbed for ˜30 minutes. If no agglutination occurs, RBCs fall into a pellet in the bottom of the well (FIG. 15A), while agglutination results in a rosette of RBCs cross-linked by bacteria (FIG. 15B). Dynamic conditions were achieved with on-slide agglutination assays, in which the suspension of RBC and bacteria were rocked on a slide surface at ˜3 sec⁻¹ and agglutination was indicated by clumping of RBCs by bacteria (FIG. 15C).

Type I fimbriated bacteria expressing the FimH-f18 variant were unable to mediate RBC agglutination in the static rosette-formation assay, even at the highest concentration of bacteria used (10⁹ bacteria/ml, see FIG. 15A). In contrast, this FimH variant was able to readily agglutinate RBCs in the dynamic rocking assay (FIG. 15B), where RBCs formed tight clumps in 42±3 seconds at the highest concentration of bacteria (10⁹ bacteria/ml), and still formed aggregates when the bacteria were ten-fold diluted. Interestingly, however, the aggregates formed by the FimH-f18 bacteria began to dissipate within 3 min after rocking was stopped (FIG. 15C) but reformed promptly if rocking was restarted. We have thus defined the dynamic conditions under which the FimH-f18 variant agglutinates RBCs, and demonstrated specifically that alternating flow will cause agglutination.

### Example 22

To illustrate that the adhesin FimH and 1Man immobilized on synthetic surfaces can be used as shear-activated nano-glue, we show that polystyrene beads coated with 1Man (see Methods) bind in a force-activated manner to a carpet of fimbriae. FIG. 11 confirms shear-activated binding of 1Man-coated 6 μm polystyrene (PS) beads to a carpet of fimbriae. The average off-rate of the 1Man beads decreased with increasing shear from 0.03 to 0.1 pN/μm2, indicating shear activation. These data also confirm that other properties of RBCs do not contribute to shear activation, and that 1Man is responsible for shear-activation. This is important to know, since RBCs have complex membranes exposing a variety of receptors and ligands on their surfaces, and have a compliant membrane from which membrane tethers can be pulled (Evans, E. et al. 1996).

### Example 23

Use of FimH or other adhesins to control the aggregation of particles, for example ionic and/or molecular scavengers by shear

Having methods available by which molecules, particles, or devices can be switched from weakly adhesive to strongly adhesive is promising for a wide range of applications in material sciences, chemical processing, separation technologies, waste management, and biotechnology. In this application, molecules or particles are used to serve a dual function: first, they are designed to scavenge pollutants, toxins, rare drugs, or other targets from fluids either via specific or non-specific binding. Second, in order to concentrate the solutes bound to the target molecules or particles, shear will be used to induce their aggregation (see FIG. 16). The aggregates can then easily be separated from the remaining solutes by sedimentation, filtration, magnetically or by the use of other methods, including bioseparation. If needed, the aggregates can be stabilized through cross-linking procedures.

The advantage of our approach is that the molecules or particles are mixed well with the solutes, at first, and do not aggregate while binding to the target chemicals. Aggregation reduces the total surface area that is available for binding with the solutes of interest. Once they are loaded with their target molecules or particles, they are aggregated by shear, thus concentrating the harvest. For this application, the target molecules or particles are engineered such that they contain shear-activated adhesins and/or their ligands, potentially in addition to other surface functionalities that can bind specifically to solutes, including ions, molecules and particles.

### Example 24

Use of FimH or other adhesins and their respective ligands to fabricate devices for particle or cell sorting applications

Many applications require that particles or cells are separated on the basis of their charge, mass, size, or other features. Here we take advantage of adhesins to sort particles or cells according to their size and/or shape, and/or surface specific ligands and/or receptors. The surface of the sorting device is either functionalized with (a) ligands that bind specifically to the adhesin exposed on the surface of target cells or particles, or (b) with adhesins that bind specifically in a shear-dependent manner to ligands exposed on the surface of the cells or particles of interest. Cells or particles that carry the ligand and/or adhesin can be separated from other particles or cells. Furthermore, the total force acting on the cells or particles in the vicinity of the device wall increases with both the shear flow and the hydrodynamic cross section of the cell or particle. Thus, shear flow conditions can be adjusted to specifically select one hydrodynamic cross section versus larger or smaller cross sections.

The sorting device can include, for example, a parallel plate flow chamber, or a microfluidic system. The surfaces in the flow chamber do not necessarily have to be parallel to each other. In addition to functionalizing the device surface with ligands or receptors, respectively, the device may contain other features that help in the pre-sorting, sorting and/or subsequent analysis of cell or particles, and/or their content. For example, one can choose the flow conditions such that the cells or particles of interest firmly adhere to the device surface thereby separating the target cells or particles from the remainder. A change in flow conditions can then release the target cells or particles from the surface for further downstream processing and/or analysis.

We immobilized bacteria expressing the FimH adhesin on the surface of a flow chamber coated with bovine RNAseB, a model tri-mannose-containing receptor substrate to probe the flow regime in which red blood cells (RBC) tightly bind to the surface of the device (Thomas 2002). The outer membrane of the RBC presents the ligand that binds specifically to the FimH adhesin.

Protocol: In brief, RBCs were allowed to bind to the bacterial carpet, and the unbound cells were removed by rinsing under moderate fluid flow shear (0.28 to 0.90 dynes/cm²). RBCs that remained attached to the bacterial carpet were then subjected to various shear conditions. As observed in the agglutination assays, the original attachment of RBCs to bacteria could be inhibited by 50 mM a-methyl-mannoside, indicating that the bacteria-RBC interactions were FimH mediated.

Findings: Under low shear conditions (from 0.01 to 0.14 dynes/cm² shear stress), the RBCs attached to the FimH-expressing bacteria (FimH-f18) were found to bind weakly, such that the cells moved sporadically along the adhesive surface (FIG. 17A). However, at moderate shear (0.28 to 0.90 dynes/cm2), these RBCs exhibited decreased mobility (FIG. 17D) eventually becoming firmly adhered to the FimH-f18 bacterial carpet (FIG. 17C). Not only did fewer cells move at moderate shear, but those that did, moved more slowly. When the shear was switched back and forth repeatedly between low and moderate shear levels, the cells started and stopped moving repeatedly, indicating that the process of adhesion enhancement under shear was reversible. Thus, the FimH-f18-mediated adhesion of bacteria to RBC is stronger under moderate shear than under low shear, i.e., is shear-dependent. At sufficiently high shear (>2 dynes/cm²), RBCs began to move on the bacteria expressing either FimH variant (FIGS. 17C-D) and at shears much higher than 10 dynes/cm², all RBCs detached from the bacterial carpet. Thus, the flow chamber results corresponded well to the RBC agglutination patterns, with both series of experiments indicating that FimH-f18 mediates stronger binding of bacteria to RBCs under high shear than under low shear conditions. In particular, this experiment demonstrated that continuous flow can cause strong binding of adhesins to their ligands.

### Example 25

We immobilized fimbriae from bacteria expressing FimH on the surface of a flow chamber. First the fimbriae were purified by shearing the fimbriae off the bacterial cells followed by purification using ultra-centrifugation and salt precipitation. Then the concentrated fimbriae solution (2 mg/ml by protein) was diluted 500× and deposited on the surface of the flow chamber as described by Sokurenko et al, 1995. RBCs were allowed to bind on the fimbrial carpet, and the unbound RBCs were washed off as in the first proof of principle. The test were conducted in the same conditions as in the first proof of principle, and demonstrated that purified fimbriae, like whole bacteria, bind to RBCs stronger in the presence of shear.

### Example 26

Either RBCs, for comparison to our previous studies (Thomas et al, 2002), or polystyrene beads coated with 1Man or 3Man were allowed to bind to the E. coli or fimbriae coated plates (FIG. 11). The detachment properties of the remaining cells were measured at varying levels of shear. The binding pattern of RBCs is essentially the same when they attach to purified fimbriae and when they attach to bacteria: At low shears (from ˜0.001 to ˜0.01 N/m²) the RBCs bind weakly to the fimbriae carpet (off-rates of k_{off}=3×10⁻³ s⁻¹) and move at average speeds of up to 0.3 μm/s. At moderate shear (0.02 to 0.1 N/m²), the RBCs bind more tightly to the fimbriae: their off-rate drops an order of magnitude to under k_{off}=2 10−4 s⁻¹, and the average speed drops to under 0.05 μm/s. At higher shears the RBCs roll similarly on fimbriae as on a bacterial carpet. The similarity between the binding patterns of RBCs over bacteria and over fimbriae shows that the presence of fimbriae is sufficient for shear enhanced binding.

### Example 27

To further illustrate shear-enhanced adhesion in separation experiments, we sorted particles in different regions of a fluid device according to particle size (FIG. 13). Our flow chamber had two regions differing in width by a factor of 4 (one is 2.5 mm and the other 10 mm wide), making the shear stress 4-fold smaller in the wider region. A mixture of 3 μm and 6 μm beads was seeded in both regions, and after five minutes of exposure, the large beads kept holding on to the surface in the low shear region while they rolled away in the high shear region. As expected, the small beads did the opposite, they rolled away in the high shear regions and firmly attached in the low shear regions.

### Example 28

Use of FimH or other adhesins and their respective ligands to shear-activate the assembly of molecules, particles and micro/nanosystems into novel materials and devices.

Recent technological advances have made it possible to engineer materials and devices on the nanometer length scale through the exploitation of self-assembly processes. This will enable a new generation of materials and devices, since self-assembly processes enable the integration of many dissimilar molecules or particles into one material or device such that the material or device has a number of complex functions.

One problem that can be solved by the use of shear-activated adhesins is the following: it is critical in the manufacturing processes of these above envisioned nanoscale materials and devices that premature assembly or aggregation of its constituents is suppressed until all constituents are well mixed and in a controlled position. The onset of shear flow is then used to induce their spontaneous adhesion to each other. A shear-induced self-assembly processes is thereby initiated. In order to fix the relative position of all constituents, the shear flow activation is followed by a cross-linking reaction using chemical or optical procedures, including various cross-linking chemistries or photo polymerization.

As discussed above, at least some of the molecules or nanoparticles that serve as nanoscale building blocks will carry in addition to their own functionalities an adhesin or the corresponding ligand. In an alternative embodiment, shear flow can first be used to create long-range patterns made in a solution of one or more dissimilar molecules and/or nanoparticles. Upon shear-activation, the spontaneous self-assembly of the constituents is induced. Again, as described above, flow-induced patterns can then be stabilized through a cross-linking step.

Two geometries are shown in FIG. 18 to illustrate how the shape of systems A and/or B, respectively, can be used to assemble materials or devices of interest. The first consists in the self-assembly of a membrane in solution. “Pancake” shaped particles, whose edges contain adhesin and ligand, are exposed to shear and aggregate to form flat layers (see FIG. 18). Similarly, rod shaped particles whose ends contain adhesin and ligand are exposed to shear and aggregate, forming chains (FIG. 18). As discussed above, these patterns can be stabilized through a cross-linking step if necessary. Finally, a surface containing FimH or other adhesin or the complementary ligand will bind these particles under shear and can be used to retain the particles during washing steps, but will release them into a new solution after the flow is stopped. Other geometries where various parts of a microparticles are selectively coated with FimH or other adhesin are also within the scope of this invention.

### Example 29

Use of FimH or other adhesins and their respective ligands for drug delivery or as part of carriers to address regions of high shear in the cardiovascular system, urinary track, or in man-made fluidic systems

Many diseases, including cardiovascular diseases, result in major changes of flow rates and shear. For example, deposits can narrow the channel diameter of blood vessels or of any man-made fluidic system. Common approaches in medicine and in industry rely on invasive tools, like the microrotor, for the removal of deposits. While the use of microrotors has become common medical practice, it has lately been suggested that the resulting debris in blood vessels may lead to brain damage and other side effects. Having access to non-invasive tools would thus constitute a major medical and industrial advance.

We propose to target these constricted areas by injecting shear-dependent drug carriers that can selectively bind to only those vessel walls along which the shear exceeds a critical threshold value. The flow conditions, for example, can be probed by Doppler Ultrasound to optimize the conditions for this non-invasive treatment. Drug carriers can then be used to deliver drugs in a shear-dependent manner.

Another application is that mineral deposits often occur on the surfaces of synthetic heart valves, stents, and other biomedical implants thus compromising their function. Mineral deposits can either lead to vessel constriction, or in the heart to turbulent flow. Again, drug carriers that show shear-activated surface adhesion can deliver drugs locally and therefore in elevated concentrations, which can degrade the deposits.

### Example 30

Another embodiment includes shear-directed assembly of particles on controlled surface regions. One approach is to pattern the surface with either the receptor or ligand. In a sequential step, particles will be accumulated on these designated surface spots by coating the particles with ligands and receptors simultaneously. The particles will then bind to the surface and aggregate among themselves in a shear-dependent manner to form larger aggregates in designated areas. The particle size can range from the nanoscale to macroscopic. Alternatively, another receptor or ligand could be patterned to the surface to immobilize nucleation sites for the particles. For example, biotin or streptavidin can be patterned on the surface to hold a particle that is functionalized with the complement to biotin or streptavidin, and simultaneously with receptors and/or ligands that show shear activation. Nucleation will then happen only around these particles under a shear regime that is dependent on the particle/aggregate size.

### Example 31

Use of FimH or other adhesins and their respective ligands for the fabrication of shear-activated microvalves

Microfluidic systems are important in many applications. In particular, they are very important in biomedical research, combinatorial chemistry, and clinical diagnostic systems. Considerable interest exists in mechanically actuated microvalves. For example, microfluidic on-off valves, switching valves, and pumps have been built with multilayer soft lithography utilizing the pressure in cross-channels to close channels. Alternatively, surface patterning has been used to form valves that resist wetting but can be opened by pressure above a critical value. Furthermore, microplugs have been fabricated that can be moved within the channels thereby opening or closing channels of interest using electric or magnetic fields.

The valves of this invention do not require that the channel diameter is compressed through the application of external pressure, nor do they require movable parts that constrict the flow on demand. They also do not have any seals that can break during operation. In contrast to the construction principles of common valves, the principle of our invention is that agglutination of particles in the fluid flow or binding of particles to the channel walls leads to a partial or complete constriction of the channel. The constriction is reversible as the shear is reduced. The invention builds upon prior observations made by studying the agglutination of RBCs in the presence of bacteria as model particles under shear. The agglutination can be induced by increasing the fluid flow. If a slow flux through the apparatus is desired, a fast alternating flow can be used with a slow net forward component in order to create high shear without a high throughput.

Several examples of valves are shown here, but it is to be understood that the agglutination of particles and/or particle sticking to the channel walls in a narrow channel is the essence of this application, and that our invention can potentially be used also in combination with already existing technology. Moreover, there is the option to add the particles to the fluid as in FIG. 20, or to recirculate the particles with recirculating technology other than the one shown below (FIG. 19).

A shear-activated microvalve can act as a pressure-compensator, and regulate the flow of fluid through a channel so that the flow rate remains nearly constant with pressure, as seen in FIG. 19. The addition of a narrow bypass channel results in a switching valve, as seen in FIG. 19. Finally, using external forces to create agitation within the valve allows to the partial or complete valve closure via shear-activated aggregation as seen in FIG. 20.

### Example 32

Use of FimH or other adhesins and their respective ligands as force enhanced adhesives.

Pressure sensitive adhesives are important in industry because they allow the user to control when the adhesive is activated. They are used in various applications ranging from semiconductor manufacturing to construction, and diaper closure tapes and other tapes, labels and films.

The adhesives for these purposes adhere only when two films are sheared by the user. The films contain FimH or other shear-activated adhesins, and complementary ligands. Ligand and adhesin can be on separate complementary films, mixed on the same film, or/and mixed with other adhesives.

In this application it is proposed to use either whole fimbria to which FimH is attached in bacteria, or a subunit of the fimbriae such as FimH, or any other adhesin, to bind to a respective ligand in order to achieve adhesion. We have already demonstrated that FimH mediates shear-activated adhesion in aqueous conditions, but this idea is intended to include the possibility of the adhesive working in dry environments as well (air is a fluid). Because bacteria are exposed to many extreme environments, fimbriae are resilient and even resist proteases, so denaturation in various dry or aqueous environments is unlikely. Adhesion between purified fimbriae and RBCs has been observed in this lab in recent experiments as described above.

Some advantages over traditional adhesives would be activation of the adhesive when desired (for alignment purposes for example), reversibility of the sticking when force is removed, ability to work in aqueous environments, and reusability. Its function can also be to temporarily hold the films while a second adhesive with a relatively long setting time (compared to the time for most the FimH-ligand bonds to break) sets.

### Example 33

Use of antibodies to selectively block the shear-enhanced binding of adhesins on demand

For several medical and technological applications it is of considerable interest to suppress on demand the shear-activation of adhesins from low to high. Antibodies can be used to block shear-activation, for example of FimH. Antibodies are thereby directed against those amino acid sequences of the adhesin that are involved in the structural changes leading to its activation from low to high. Once the antibody binds to the adhesin, it stabilizes the structure of the adhesin in the low-affinity state, thereby suppressing the shear-activation of the high affinity state. This, in turn prevents strong attachment of the bacteria to target cells or surfaces, or of other molecules or particles that carry adhesins to their respective target cells or surfaces.

Beyond the applications discussed above, this method has major implications in the treatment and/or prevention of diseases, such as urinary tract infections. Such an approach to the design of vaccine is much more effective than other attempts that try to block the receptor-binding region to prevent bacterial adhesion, for example by the use of antibodies or polypeptides. Furthermore, some parts of receptor-binding amino acid regions might be hidden or structurally buried under static, non-dragging conditions and become exposed only in the force-induced conformation. In many cases, this prevents effective interaction of the specific antibody with these regions. However, amino acid regions that are involved in the structural changes that lead to the high-affinity conformation of the adhesin under shear are distinct from the receptor-binding ones and are accessible to the antibodies under any conditions. Therefore, antibodies against these regions will provide better protection than antibodies against the receptor-binding ones.

Currently, clinical trials are under way for vaccines based on the purified lectin domain of the FimH protein and FimH complex with the molecular chaperone FimC. The vaccine preparation is produced by MedImmune, Inc., Maryland, USA and is directed primarily towards treatment and prevention of urinary tract infections. Though the preliminary studies on mice and primates have shown the vaccine to be effective, the molecular mechanism of its action remains unclear. It was assumed that the main protective antibodies are invoked against the receptor-binding region of FimH, but experimental proof that the antibodies do indeed bind to the receptor-binding site has not been obtained. Still, a patent has been filed by Langermann, S., and Hultgren, S. (United States) CA 2379069 ‘FIMH ADHESIN-BASED VACCINES’ for the use of peptides that correspond to the receptor-binding region of FimH as vaccine, e.g., positions 1-20, 46-54 and 127-148. In contrast, we claim that the above-mentioned antibodies bind to other regions of FimH that play a role in the shear force-activation. These regions away from the receptor-binding site include, but are not limited to, positions 150-160 (interdomain linker chain loop) and positions 25-31 and 110-123 (both are linker chain stabilizing loops).

TABLE 2

| form of surface- | | |
|---|---|---|
| immobilized | Soluble | Inhibition by anti-FimH |
| adhesin | receptor target | antiserum (dilution 1:500) |
| Lectin domain | HRP (1 mg/ml) | 5% |
| Fimbriated bacteria | Buccal cells | 95% |
| | (100 cells/mm3) | |

Patent applications proposing the use of FimH-based vaccines by targeting the receptor site:

1) Patent Application CA 2379069 ‘FIMH ADHESIN-BASED VACCINES’ claiming an immunogenic composition comprising a purified polypeptide corresponding to a mannose-binding portion of FimH to be used against the urinary tract infection caused by E. coli. (Langermann et al 1997)

2) Patent Application CA 2180726 ‘RECEPTOR SPECIFIC BACTERIAL ADHESINS AND THEIR USE’ claiming invention of a method of targeting a non-adhesin compound (including vaccine peptides) to a specific location recognized by bacterial adhesins.

SEQ ID NO:1 is the sequence of E. coli FimH amino acids 25-31, APAVNVG.

SEQ ID NO:2 is the sequence of E. coli FimH amino acids 110-123, TPVSSAGGVAIKAG.

SEQ ID NO:3 is the sequence of E. coli FimH amino acids 150-160, ANNDVVVPTGG.

SEQ ID NO:4 is the sequence of E. coli FimH amino acids 25-32, APAVNVGQ.

SEQ ID NO:5 is the sequence of E. coli FimH amino acids 110-124, TPVSSAGGVAIKAGS.

SEQ ID NO:6 is an artificial sequence of E. coli FimH amino acids 25-32 with a substitution at position 32, APAVNVGL.

SEQ ID NO:7 is an artificial sequence of E. coli FimH amino acids 110-124 with a substitution at position 124, TPVSSAGGVAIKAGA.

SEQ ID NO:8 is an artificial sequence of E. coli FimH amino acids 110-160 with a substitution at position 154, ANNDPVVPTGG.

SEQ ID NO:9 is an artificial sequence of E. coli FimH amino acids 110-160 with a substitution at position 155, ANNDVPVPTGG.

SEQ ID NO:10 is an artificial sequence of E. coli FimH amino acids 110-160 with a substitution at position 156, ANNDVVPPTGG.

SEQ ID NO:11 is an artificial sequence of E. coli FimH amino acids 110-160 with substitutions at positionsl54 and 155, ANNDPPVPTGG.

SEQ ID NO:12 is an artificial sequence of E. coli FimH amino acids 110-160 with substitutions at positionsl 55 and 156, ANNDVPPPTGG.

SEQ ID NO: 13 is an artificial sequence of E. coli FimH amino acids 110-160 with substitutions at positions 154-156, ANNDPPPPTGG.

SEQ ID NO: 14 is a sequence of a nascent E. coli FimH protein.

MKRVITLFAVLLMGWSVNAWSFACKTANGTAIPIGGGSANVYVNLAPAVN
VGQNLVVDLSTQIFCHNDYPETITDYVTLQRGSAYGGVLSSFSGTVKYNG
SSYPFPTTSETPRVVYNSRTDKPWPVALYLTPVSSAGGVAIKAGSLIAVL
ILRQTNNYNSDDFQFVWNTYANNDVVVPTGGCDVSARDVTVTLPDYPGSV
PIPLTVYCAKSQNLGYYLSGTTADAGNSIFTNTASFSPAQGVGVQLTRNG
TIIPANNTVSLGAVGTSAVSLGLTANYARTGGQVTAGNVQSIIGVTFVYQ

SEQ ID NO: 15 is a sequence of a mature (N-terminal 21 amino acids cleaved) E. coli FimH protein. The mature protein is used for assigning amino acid positions.

FACKTANGTAIPIGGGSANVYVNLAPAVNVGQNLVVDLSTQIFCHNDYPE
TITDYVTLQRGSAYGGVLSSFSGTVKYNGSSYPFPTTSETPRVVYNSRTD
KPWPVALYLTPVSSAGGVAIKAGSLIAVLILRQTNNYNSDDFQFVWNIYA
NNDVVVPTGGCDVSARDVTVTLPDYPGSVPIPLTVYCAKSQNLGYYLSGT
TADAGNSIFTNTASFSPAQGVGVQLTRNGTIIPANNTVSLGAVGTSAVSL
GLTANYARTGGQVTAGNVQSIIGVTFVYQ

SEQ ID NO: 16 is a an artificial sequence of a consensus DNA sequence encoding E. coli FimH.

ATGAAACGAGTTATTACCCTGTTTGCTGTACTGCTGATGGGCTGGTCGGT
AAATGCCTGGTCATTCGCCTGTAAAACCGCCAATGGTACCGCAATCCCTA
TTGGCGGTGGCAGCGCCAATGTTTATGTAAACCTTGCGCCTGCCGTGAAT
GTGGGGCAAAACCTGGTCGTAGATCTTTCGACGCAAATCTTTTGCCATAA
CGATTACCCAGAAACCATTACAGACTATGTCACACTGCAACGAGGTTCGG
CTTATGGCGGCGTGTTATCTAGTTTTTCCGGGACCGTAAAATATAATGGC
AGTAGCTATCCTTTCCCTACTACCAGCGAAACGCCGCGGGTTGTTTATAA
TTCGAGAACGGATAAGCCGTGGCCGGTGGCGCTTTATTTGACGCCGGTGA
GCAGTGCGGGGGGAGTGGCGATTAAAGCTGGCTCATTAATTGCCGTGCTT
ATTTTGCGACAGACCAACAACTATAACAGCGATGATTTCCAGTTTGTGTG
GAATATTTACGCCAATAATGATGTGGTGGTGCCCACTGGCGGCTGCGATG
TTTCTGCTCGTGATGTCACCGTTACTCTGCCGGACTACCCTGGTTCAGTG
CCGATTCCTCTTACCGTTTATTGTGCGAAAAGCCAAAACCTGGGGTATTA
CCTCTCCGGCACAACCGCAGATGCGGGCAACTCGATTTTCACCAATACCG
CGTCGTTTTCACCCGCGCAGGGCGTCGGCGTACAGTTGACGCGCAACGGT
ACGATTATTCCAGCGAATAACACGGTATCGTTAGGAGCAGTAGGGACTTC
GGCGGTAAGTCTGGGATTAACGGCAAATTACGCACGTACCGGAGGGCAGG
TGACTGCAGGGAATGTGCAATCGATTATTGGCGTGACTTTTGTTTATCAA
TAA

In the presence of shear flow, E. coli bacteria show a shear-dependent biphasic accumulation on 1Man surfaces that has not been previously documented. Bacterial adhesion increases with shear stress until it peaks and drops off at eventually high shear (FIG. 6) whereas the accumulation rate steeply drops with increasing shear. The shear-enhanced accumulation is thus not due to an enhanced rate of binding but an increased lifetime of bacteria in the surface-bound state. Bacterial accumulation peaks at a physiologically relevant shear stress. The biphasic behavior of E. coli which is mediated by FimH binding to 1Man parallels the biphasic dependence of the lifetime as function of force found for p-selectin bound to the mucin (Marshall, 2003). Once bound to 1Man surfaces, E. coli can exist in two states. It either binds firmly or rolls along the surface. The ratio of rolling to stationary cells is small at low and high shear, and peaks at a shear stress (FIG. 7), while the total number of surface-bound bacteria increases with shear stress. Bacteria occasionally switch between the two states, from rolling to stationary or vice versa. Once the shear stress at which maximal accumulation is observed is switched to lower shear, the bacteria are washed off the surface (FIG. 8A). The rolling cells detach at an exponential rate (FIG. 8B). The stationary bacteria gradually convert into the rolling state with a linear dependency. If the shear stress is switched from optimal accumulation to higher values, the bacteria firmly adhered (FIG. 8C). This is most interesting, since at these high shear stresses, the accumulation rate is rather low. Accordingly, this high shear regime inhibits initial adhesion, however, prolongs the lifetime of those bacteria that are already bound to the surface. The transition from rolling to stationary adhesion is due to an increase in drag force acting on the bacteria rather than fluid velocity (FIG. 8D) and the transition is reversible.

In addition to altering the shear dependence of RBC adhesion, the mutations tested above affected the affinity of FimH for Man1 receptors. The V156P substitution increased Man1 affinity when introduced into both FimH variants, while the Q32L/S124A substitutions decreased Man1 binding of both FimH-j96 and FimH-f18 to an almost undetectable level (Table 1, C and D). Neither mutation affected FimH trimannose binding (Table 1, C and D). Importantly, the Man1 binding capability of all FimH variants tested correlated directly with their ability to agglutinate RBCs under static conditions (FIG. 5A). Furthermore, in addition to the A27V substitution, other naturally occurring mutations as well as some induced mutations that enhance Man1 binding to varying degrees also correspondingly enhance RBC agglutination in static conditions (Sokurenko et al., 1998, 2001). Remarkably, some of the Man1-enhancing mutations identified previously (e.g., A25P, A118V, and the 117G-120I deletion) are located within or immediately adjacent to the region of linker-stabilizing bonds, while most of the remaining functional mutations map to the interdomain region of the lectin as well as the pilin domain of FimH (Schembri et al., 2000, Sokurenko et al., 2001). These mutations therefore should affect different steps in the force-induced conformational changes discussed above. Taken together, the studies presented here show that the change in the affinity toward Man1 and the shear dependence of FimH are concurrent processes.

Based on the sum of these observations, and without wishing to be bound to any particular theory, we postulate an emerging model for E. coli adhering to surfaces presenting 1Man as follows: the initial attachment is dominated by short-lived bonds that show slip-bond character that weaken upon increased shear stress. Once surface-bound, tensile forces acting on the receptor-ligand bonds switch at least a fraction of the bonds to a long-lived state with catch-bond characteristics. The stationary state of E. coli is long-lived for seconds, minutes or hours at constant flow rates. Previous SMD simulations proposed a model how a mechanical perturbation within the lectin domain of FimH might switch the adhesin from low to high affinity. Transitioning from the stationary to the rolling state requires that the long-lived high-affinity bonds convert back to short-lived low-affinity bonds and we measured a linear decay rate for whole bacteria. The kinetics by which single stretched FimH adhesins can refold back to the low-affinity state are not yet known. The rolling state is presumably a collection of short-lived bonds. These short-lived low-affinity bonds decay exponentially, as expected for slip-bonds. Taken together, our data suggest a model whereby force induces a high-affinity conformation of the FimH-1Man bond that has long lifetimes. More precisely, while the FimH-1Man bond is generally of low affinity, it can transition to a high-affinity state and the probability of this transition is dramatically enhanced by force.

The finding that E. coli accumulation on Man I-coated surfaces can be increased 100-fold at a shear rate of 5 dynes/cm², which is within the physiological range in many compartments in the human body, is of considerable physiological significance. While several studies have suggested that shear may slightly enhance bacterial binding (D. E. Brooks and T. J. Trust (1983); Z. J. Li, et al. (2000); N. Mohamed et al. (2000)), none of these studies found that a lack of shear could prevent bacterial surface accumulation or cause massive bacterial detachment. FimH is the most common adhesin on enteric bacteria and has been studied for decades, yet this phenomenon has not been observed in traditional assays. Many bacteria and cells adhere under shear: FimH is only one of the adhesion molecules useful in this invention. We have shown that shear stress induces a switch from rolling to stationary adhesion. These behaviors have physiological significance for bacteria attempting to leave, expand or roll into, or remain in, particular niches in a shear-dependent manner. In particular, the natural niche of commensal E. coli—the intestines—is exposed to high levels of shear stress due to both peristalsis and high viscosity that favor accumulation. Both rolling and stationary adhesion to 1Man is mediated by FimH, and FimH is the only mannose-binding protein in the genome of the E. coli variants used in these studies. Until now, it has been assumed that if a single bond type can mediate both rolling and stationary adhesion, the stationary adhesion always required lower shear stress rather than higher (K. C. Chang et al. 2000). In experiments where shear enhances adhesion such as the rolling of lymphocytes on selectins (M. B. Lawrence et al. 1997), stationary adhesion is only observed when a second class of adhesion proteins, integrins, also becomes involved (J. J. Campbell et al., 1998).

This invention has been illustrated and explained in terms of numerous specific examples; however, as will be appreciated by those skilled in the art, equivalent adhesion molecules, surfaces, devices, and means for producing force-activated bond stress may be substituted for those specifically described, and are included within the scope of the appended claims.

We claim:

1. A method for changing binding strength of an isolated force-activated bond stress-dependent adhesion molecule (I-FABSDAM) to a force-activated bond stress-dependent binding ligand (FABSDB-L) for said I-FABSDAM, said method comprising changing a bond stress on said I-FABSDAM wherein said binding strength increases when said bond stress increases and decreases when said bond stress decreases.

2. The method of claim 1 wherein said bond stress is caused by a shear force.

3. The method of claim 1 wherein said bond stress is a tensile force.

4. The method of claim 1 comprising increasing said bond stress whereby said binding strength is increased.

5. The method of claim 1 comprising decreasing said bond stress whereby said binding strength is decreased.

6. The method of claim 2 wherein said method results in said I-FABSDAM being tightly bound to said FABSDB-L.

7. The method of claim 1 wherein said I-FABSDAM is selected from the group consisting of adhesins, selectins, integrins, cadherins, immunoglobulin superfamily cell adhesion molecules, and syndecans that are capable of binding in a force-activated bond stress-dependent manner.

8. The method of claim 7 wherein said adhesin comprises a FimH polypeptide, or the lectin domain of a FimH polypeptide.

9. The method of claim 8 wherein said FimH polypeptide is an E. coli FimH polypeptide.

10. The method of claim 6 wherein said I-FABSDAM comprises a polypeptide having the sequence of Genbank Accession Number P08191.

11. The method of claim 6 wherein said FimH polypeptide is engineered.

12. The method of claim 6 wherein said FimH polypeptide is a FimH-f18 polypeptide derived from the E. coli strain f18.

13. The method of claim 12 wherein said FimH-f18 polypeptide is engineered to comprise a valine at amino acid position 27.

14. The method of claim 6 wherein said FimH polypeptide comprises an amino acid substitution selected from the group consisting of a proline at position 154, a proline at position 155, a proline at position 156, a leucine at position 32, and an alanine at position 124.

15. The method of claim 8 wherein said FimH polypeptide is FimH-j96.

16. The method of claim 1 wherein said FABSDB-L comprises mannose or fructose.

17. The method of claim 16 wherein said mannose is selected from the group consisting of monomannose, trimannose, and oligomannose.

18. The method of claim 1 wherein said I-FABSDAM is attached to a particle selected from the group consisting of bacterial pili, naturally occurring isolated molecules, synthetic molecules, proteins, polypeptides, organelles, prokaryotic cells to which said I-FABSDAM is not native, eukaryotic cells to which said I-FABSDAM is not native, viruses, organisms, nanoparticles, and microbeads, microparticles, or a surface selected from the group consisting of cell membranes, device surfaces and a synthetic substrate surfaces.

19. The method of claim 18 wherein said FABSDB-L is also attached to said particle.

20. The method of claim 1 wherein said FABSDB-L is attached to a particle selected from the group consisting of bacterial pili, isolated molecules, synthetic molecules, proteins, polypeptides, organelles, prokaryotic cells to which said I-FABSDAM is not native, eukaryotic cells to which said I-FABSDAM is not native, viruses, organisms, nanoparticles, microparticles and microbeads or a surface selected from the group consisting of cell membranes, device surfaces and synthetic substrate surfaces.

21. The method of claim 20 wherein said I-FABSDAM is also attached to said particle.

22. The method of claim 1 wherein changing said bond stress comprises applying a bond stress between a bond stress dependence lower threshold of said I-FABSDAM and a bond stress dependence upper threshold of said I-FABSDAM.

23. The method of claim 1 wherein changing said bond stress comprises applying a bond stress between about 0.01 dynes/cm² and about 100 dynes/cm².

24. The method of claim 1 wherein changing said bond stress comprises applying a bond stress between about 0.05 dynes/cm² and about 20 dynes/cm².

25. The method of claim 1 wherein changing said bond stress comprises applying a bond stress between about 0.1 dynes/cm² and about 10 dynes/cm².

26. The method of claim 1 applied to a system wherein a first component of said system comprises a plurality of said I-FABSDAMs attached to a first object, wherein a second component of said system comprises a plurality of said FABSDB-Ls attached to a second object, and wherein said I-FABSDAMs and FABSDB-Ls are capable of binding to each other in a force-activated bond stress-dependent manner, and wherein said method comprises increasing bond stress on said I-FABSDAMs, resulting in said first component changing from being unbound to said second component to being bound to said second component.

27. The method of claim 1 applied to a system wherein a first component of said system comprises a plurality of said I-FABSDAMs attached to a first object, wherein a second component of said system comprises a plurality of said FABSDB-Ls attached to a second object, and wherein said I-FABSDAMs and FABSDB-Ls are capable of binding to each other in a force-activated bond stress-dependent manner, and wherein said method comprises decreasing bond stress on said I-FABSDAMS, resulting in said first component changing from being bound to said second component to being unbound from said second component

28. The method of claim 1 applied to a system wherein a first component of said system comprises a plurality of said I-FABSDAMs attached to first particles, and a second component of said system comprises a plurality of said FABSDB-Ls attached to second particles, said method comprising homogenously mixing said first and second components, then increasing said bond stress on said system, whereby a substantially uniform material comprising complexes of said first components with said second components is formed.

29. The method of claim 28 further comprising cross-linking said substantially uniform material once said complexes have been formed by increasing said bond stress.

30. The method of claim 1 wherein a plurality of said I-FABSDAMs are attached to a first selected surface of a plurality of first selected three-dimensional forms, wherein a plurality of FABSDB-Ls are attached to second selected surface of a plurality of second selected three dimensional forms, and wherein said changing is increasing, thereby resulting in said first and second forms self-assembling into a selected geometric pattern.

31. The method of claim 30 wherein said first form is the same as said second form.

32. The method of claim 31 wherein said first and second forms are cylinders, wherein said first and second surfaces are the curved sides of said cylinders, and said geometric pattern is a layer composed of said cylinders.

33. The method of claim 32 wherein said layer is a synthetic membrane.

34. The method of claim 30 wherein said first and second forms are cylinders, wherein said first and second surfaces are the flat ends of said cylinders, and said geometric pattern is a chain composed of said cylinders.

35. The method of claim 34 wherein said first form has said I-FABSDAMs attached thereto but does not have FABSDB-Ls capable of binding said I-FABSDAMs attached thereto;
wherein said second form has said FABSDB-Ls attached thereto but does not have FABSDAMs capable of binding said FABSDB-Ls attached thereto; and said geometric pattern is an alternating link chain.

36. The method of claim 30 wherein said first and second forms are cylinders, wherein each cylinder comprises a first flat end and a second flat end, wherein said first flat ends are attached to said I-FABSDAMs and said second flat ends are attached to said FABSDB-Ls, and said geometric pattern is a directional chain composed of said cylinders.

37. The method of claim 1 performed in a fluid-containing channel, wherein a plurality of said I-FABSDAMs and said FABSDB-Ls are attached to particles or surfaces and are present in an amount sufficient to clog said channel when said I-FABSDAMs and said FABSDB-Ls are bound to each other, said method comprising changing said bond stress on said I-FABSDAMs whereby said binding strength of said I-FABSDAMs and FABSDB-Ls is changed, whereby the flow rate of said fluid through said channel or the pressure drop is changed.

38. The method of claim 37 wherein said bond stress is increased causing said I-FABSDAMs and FABSDB-Ls to be bound to each other, whereby said flow rate is decreased.

39. The method of claim 37 wherein said bond stress is decreased causing said I-FABSDAMs and FABSDB-Ls to be unbound to each other, whereby said flow rate is increased.

40. The method of claim 37 wherein said I-FABSDAMs and/or said FABSDB-Ls are bound to particles.

41. The method of claim 37 wherein said I-FABSDAMs or said FABSDB-Ls are bound to a wall of said channel.

42. The method of claim 37 wherein said channel is in fluid communication with a fluid exit port and a bypass port, wherein changing said bond stress changes the amount of fluid flowing through said exit and bypass ports.

43. A method for removing a target particle from a fluid comprising:
(a) adding to said fluid a target particle binding agent, said target particle binding agent being attached to a first member of a FABSDAM/FABSDB-L pair;
(b) adding to said fluid the second member of a FABSDAM/FABSDB-L pair attached to a removing agent;
(c) allowing said target particle binding agent to bind said target particle;
(d) applying a bond stress to said FABSDAM to allow force-activated bond stress-dependent binding of said first pair member and said second pair member, thereby forming a complex comprising said target particle, said target particle binding agent attached to said first pair member, and said second pair member attached to said removing agent; and
(e) removing said complex from said fluid.

44. The method of claim 43 wherein step (e) comprises a step selected from the group consisting of sedimentation, filtration, bioseparation, applying an electric force, and applying a magnetic force.

45. The method of claim 43 wherein said target particle is selected from the group consisting of pollutant particles, toxin particles, and drug particles.

46. The method of claim 43 wherein said target particle binding agent is an antibody.

47. A method for separating first FABSDB-Ls from second FABSDB-Ls, wherein said FABSDB-Ls are in a fluid, wherein said FABSDB-Ls are capable of binding to FABSDAMs in a force-activated bond stress-dependent manner, and wherein said first and second FABSDB-Ls induce different bond stresses on said FABSDAM under the same conditions, said method comprising:
(a) contacting said fluid with an amount of said FABSDAMs sufficient to bind substantially all of said first FABSDB-Ls, wherein said FABSDAMs are attached to a removing agent;
(b) applying a bond stress to said FABSDAMs sufficient to cause binding of said first FABSDB-Ls to said FABSDAMs to form a complex, said bond stress being insufficient to cause binding of said second FABSDB-Ls to said FABSDAMs; and
(c) removing said complex comprising said first FABSDB-Ls, and FABSDAMs and said removing agent from said fluid.

48. The method of claim 47 wherein said removing agent consists of particles capable of responding to a removing force.

49. The method of claim 45 also comprising:
(a) contacting said fluid with said FABSDAMs attached to a removing agent in an amount sufficient to bind to substantially all of said second FABSDB-Ls;
(b) applying a second bond stress to said FABSDAMs sufficient to cause binding of said second FABSDB-Ls to said FABSDAMs to form a second complex; and
(c) separating said second complex comprising said second FABSDB-L from said fluid.

50. The method of claim 49 wherein said second bond stress is selected so as to cause selective binding of said FABSDAMs to said second FABSDB-Ls, to the exclusion of other components in said fluid.

51. The method of claim 47 wherein said first FABSDB-Ls differ from said second FABSDB-Ls in a characteristic selected from the group consisting of magnetic and electric charge, mass, and three-dimensional form.

52. The method of claim 47 also comprising a step of covalently linking said FABSDB-Ls to said removing agent.

53. A fluidic device comprising a surface having a plurality of I-FABSDAMs attached thereto.

54. The device of claim 53 wherein said surface is a channel wall surface or portion thereof.

55. The device of claim 53 wherein said surface is that of a component selected from the group consisting of a channel, a parallel plate flow chamber, a cylindrical channel, a microfluidic channel, and a cell sorter.

56. A method for selectively releasing into a fluid first FABSDB-Ls from a plurality of FABSDAMs to which first and second FABSDB-Ls are stress-dependently bound, and wherein when said FABSDB-Ls are bound to said FABSDAMs under bond stress, said first and second FABSDB-Ls induce different bond stresses on said FABSDAMs under the same fluid flow conditions, said method comprising:
(a) contacting said fluid with said FABSDAMs bound to said SDDB-Ls; and
(b) changing the bond stress on said FABSDAMs by an amount sufficient to cause release of said first FABSDB-Ls into said fluid, but insufficient to cause release of said second FABSDB-Ls into said fluid.

57. A method for selectively concentrating first FABSDB-Ls from second FABSDB-Ls, wherein said FABSDB-Ls are in a fluid, wherein said FABSDB-Ls are capable of binding to FABSDAMs in a force-activated bond stress-dependent manner, and wherein said first and second FABSDB-L induce different bond stresses on said FABSDAM under the same conditions, said method comprising:
(a) contacting said fluid with an amount of said FABSDAMs sufficient to bind substantially all of said first FABSDB-Ls, wherein said FABSDAMs are attached to a removing agent;
(b) applying a bond stress to said FABSDAMS sufficient to cause binding of said first FABSDB-Ls to said FABSDAMs to form a complex, said bond stress being insufficient to cause binding of said second FABSDB-Ls to said FABSDAMs; and
(c) removing said complex comprising said first FABSDB-Ls and FABSDAMS and said removing agent from said fluid.

58. A method for measuring the rate of flow of a fluid comprising:
(a) adding a plurality of FABSDAMs or FABSDB-Ls to said fluid;
(b) placing a plurality of FABSDAMs capable of binding to said FABSDB-Ls or a plurality of FABSDB-Ls capable of binding to said FABSDAMs in contact with said fluid;
(c) allowing said FABSDAMs and said FABSDB-Ls to bind in a force-activated bond stress-dependent manner; and
(d) detecting and quantitatively measuring the amount of binding of said FABSDAMs to said FABSDB-Ls;
wherein said amount of binding is indicative of the rate of flow of said fluid.

59. The method of claim 58 wherein said plurality of FABSDAMs or FABSDB-Ls placed in contact with said fluid are bound to a substrate.

60. The method of claim 59 wherein said substrate is a channel wall in contact with said fluid.

61. The method of claim 60 wherein said channel is a microchannel.

62. The method of claim 58 wherein said step of detecting and quantitatively measuring comprises measuring light scattering of said fluid.

63. A method for delivering a particle to a surface of a system, said surface having attached thereto one member of an I-FABSDAM/FABSDB-L pair, said system also comprising a fluid in contact with said surface, said method comprising:
(a) adding to said fluid the other member of said pair attached to said particle; and
(b) allowing said pair members to bind in a force-activated bond stress-dependent manner.

64. A bond stress-activated valve for controlling a fluid flow rate in a channel, said channel having a surface in contact with said fluid, said channel surface having attached thereto a plurality of a first member of an I-FABSDAM/FABSDB-L pair, said fluid comprising a plurality of the second member of said pair, wherein said first and second members are present in an amount sufficient to clog or partially clog said channel when bound in complexes in a force-activated bond stress-dependent manner.

65. The valve of claim of 64 which is a microvalve, wherein said channel is a microchannel.

66. The valve of claim of 64 wherein said fluid has a first flow rate through said channel, wherein when said first flow rate changes a bond stress on said I-FAIBSDAMs, said change resulting in a binding strength change in the binding of said I-FABSDAMs and said FABSDB-Ls, thereby changing said flow rate.

67. A bond stress-activated adhesive system comprising:
(a) a plurality of I-FABSDAMs; and
(b) a plurality of FABSDB-Ls capable of binding to said I-FABSDAMs in a bond stress dependent manner.

68. The bond stress-activated adhesive system of claim 67 wherein said I-FABSDAMs are attached to a surface of a film.

69. The bond stress-activated adhesive system of claim 68 wherein said FABSDB-Ls are also attached to said film, whereby said film is capable of adhering in a force-activated bond stress-dependent manner to itself.

70. The bond stress-activated adhesive system of claim 68 wherein said FABSDB-Ls are attached to a second film whereby said second film is capable of adhering in a force-activated bond stress-dependent manner to said first film.

71. A method for making a bond stress-activated adhesive system comprising:
(a) attaching a first member of an I-FABSDAM/FABSDB-L pair to a surface of a first film; and
(b) attaching the second member of said pair to a surface of a second film.

72. A viscosity modifier comprising a plurality of I-FABSDAMs and a plurality of FABSDB-Ls, said I-FABSDAMs and FABSDB-Ls being capable of binding to each other in force-activated bond stress-dependent manner.

73. A method for modifying the viscosity of a fluid comprising:
(a) adding to said fluid a plurality of I-FABSDAMs;
(b) adding to said fluid a plurality of FABSDB-Ls capable of binding in a shear stress-dependent manner to said I-FABSDAMs; and
(c) changing a bond stress on said I-FABSDAMs.

74. A method for interfering with the force-activated bond stress-dependent binding of a FABSDAM and a FABSDB-L capable of binding to said FABSDAM in a force-activated bond stress-dependent manner, said method comprising contacting said FABSDAM with an antibody capable of binding said FABSDAM but incapable of binding to a FABSDB-L-binding domain of said FABSDAM; and allowing said antibody to bind said FABSDAM.

75. The method of claim 74 wherein said FABSDAM is a FimH polypeptide, wherein said antibody is capable of binding to a domain of said FimH polypeptide selected from the group consisting of FimH amino acids 25-31 (SEQ ID NO: 1), FimH amino acids 110-123 (SEQ ID NO: 2), and FimH amino acids 150-160 (SEQ ID NO: 3).

76. A monoclonal antibody generated using, and capable of binding to, a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3).

77. A polyclonal antibody generated using, and capable of binding to, a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3).

78. An antibody generated using, and capable of binding to, the force-activated structure of a FABSDAM polypeptide.

79. An immunogenic composition comprising a polypeptide having an amino acid sequence selected from the group consisting of FimH amino acids 25-31 (SEQ ID NO: 1), FimH amino acids 110-123 (SEQ ID NO: 2), and FimH amino acids 150-160 (SEQ ID NO: 3).

80. The immunogenic composition of claim 79 wherein said polypeptide is produced synthetically.

81. A method for making an engineered FimH polypeptide having different force-activated bond stress-dependent binding strength to a selected FABSDB-L than a natural FimH polypeptide, said method comprising engineering a DNA sequence encoding a FimH polypeptide to encode an engineered FimH polypeptide and expressing said engineered FimH polypeptide, wherein said engineered polypeptide comprises an amino acid substitution at an amino acid position selected from positions 154-156, position 32, and position 124,

82. The method of claim 81 comprising engineering a codon selected from the group consisting of codons encoding valine at positions 154, 155, and 156 to encode proline.

83. The method of claim 81 comprising engineering the codon encoding glutamine at position 32 to encode a leucine and engineering the codon encoding serine at position 124 to encode an alanine.

84. A FimH polypeptide comprising an amino acid sequence selected from the group consisting of SEQ ID NO: 1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:l1, and SEQ ID NO:12.

85. A method for changing binding strength of an isolated force-activated bond stress-dependent adhesion molecule (I-FABSDAM) to a force-activated bond stress-dependent binding ligand (FABSDB-L) for said I-FABSDAM, said method comprising changing a bond stress on said I-FABSDAM; wherein said binding strength increases when said bond stress decreases and decreases when said bond stress increases; wherein said bond stress is between an upper force-activated bond stress-dependent threshold of said I-FABSDAM and a higher force-activated bond stress-dependent binding threshold of said I-FABSDAM.
